(19) United States
(12) Patent Application Publication
(10) Pub. No.: US 2011/0035156 A1
(43) Pub. Date: Feb. 10, 2011

(54) Method of evaluating gastric cancer, gastric cancer-evaluating apparatus, gastric cancer-evaluating method, gastric cancer-evaluating system, gastric cancer-evaluating program and recording medium

(75) Inventors: Akira Imaizumi, Kanagawa (JP); Toshihiko Ando, Kanagawa (JP); Takeshi Kimura, Kanagawa (JP); Yasushi Noguchi, Kanagawa (JP); Akira Gouchi, Okayama (JP); Hiroshi Yamamoto, Tokyo (JP)

Correspondence Address: FOLEY AND LARDNER LLP;SUITE 500, 3000 K STREET NW, WASHINGTON, DC, 20007, US

(73) Assignee: Ajinomoto Co., Inc.

(21) Appl. No.: 12/805,564

(22) Filed: Aug. 5, 2010

Related U.S. Application Data
(63) Continuation of application No. PCT/JP2009/051548, filed on Jan. 30, 2009.

(30) Foreign Application Priority Data
Feb. 6, 2008 (JP) 2008-026837

(51) Int. Cl. G01N 33/53; G06F 19/00

(52) U.S. Cl. 702/19; 436/86

(57) ABSTRACT

According to the method of evaluating gastric cancer of the present invention, amino acid concentration data on the concentration value of amino acid in blood collected from a subject to be evaluated is measured, and a gastric cancer state in the subject is evaluated based on the concentration value of at least one of Asn, Cys, His, Met, Orn, Phe, Trp, Pro, Lys, Leu, Glu, Arg, Ala, Thr, and Tyr contained in the measured amino acid concentration data of the subject.

This application is a Continuation of PCT/JP2009/051548, filed Jan. 30, 2009, which claims priority from Japanese patent application JP 2008-026837 filed Feb. 6, 2008. The contents of each of the aforementioned application are incorporated herein by reference in their entirety.

### BACKGROUND OF THE INVENTION

1. Field of the Invention

The present invention relates to a method of evaluating gastric cancer, a gastric cancer-evaluating apparatus, a gastric cancer-evaluating method, a gastric cancer-evaluating system, a gastric cancer-evaluating program and recording medium, which utilize concentration of amino acids in blood (plasma).

2. Description of the Related Art

In 2003, the number of male deaths and the number of female deaths from gastric cancer in Japan are 32846 and 17711, respectively, which is second in the total number of deaths from all cancers, is second in the total number of male deaths from cancer, and is first in the total number of female deaths from cancer.

In gastric cancer treatment, when tumor is localized to mucosa and submucosa, prognosis is well. The 5-year survival rate of gastric cancer at early stages (I to II stages) is equal to or more than 50%. In particular, the 5-year survival rate of gastric cancer at IA stage (having an invasion depth to mucosa and submucosa and no lymph node metastasis) is about 90%.

However, the survival rate is lowered with the progress of stage of gastric cancer. Early detection is important for curing gastric cancer.

Diagnosis of gastric cancer includes a pepsinogen test, X-ray examination, endoscopic examination, a tumor marker, and the like.

However, a pepsinogen test, X-ray examination, and a tumor marker do not serve as definitive diagnosis. For example, the pepsinogen test is less invasive, but the sensitivity varies in different reports, approximately from 40 to 85%, while the specificity is 70 to 85%. However, in the case of the pepsinogen test, the rate of recall for thorough examination is 20%, and it is conceived that the results are frequently overlooked. In the case of X-ray examination (indirect roentgenography), the sensitivity varies in different reports, approximately from 70 to 80%, while the specificity is 85 to 90%. However, the X-ray examination has a possibility of causing adverse side effects due to the drinking of barium, or of exposure to radiation. In the case of a tumor marker, a tumor marker which is effective for diagnosing the presence of gastric cancer does not exist at present.

On the other hand, endoscopic examination serves as definitive diagnosis, but is a highly invasive examination, and implementing endoscopic examination at the screening stage is not practical. Furthermore, invasive diagnosis such as endoscopic examination gives a burden to patients, such as accompanying pain, and there may also be a risk of bleeding upon examination, or the like.

Therefore, from the viewpoints of a physical burden imposed on patients and of cost-benefit performance, it is desirable to narrow down the target range of test subjects with high possibility of onset of gastric cancer, and to subject those people to treatment. Specifically, it is desirable that (a) test subjects are selected by a method being less invasive and having high sensitivity, (b) the target range of the selected test subjects is narrowed by subjecting the selected test subjects to gastroscopic, and (c) the test subjects who are definitively diagnosed as having gastric cancer are subjected to treatment.

Incidentally, it is known that the concentrations of amino acids in blood change as a result of onset of cancer. For example, Cynober (“Cynober, L. ed., Metabolic and therapeutic aspects of amino acids in clinical nutrition. 2nd ed., CRC Press.”) has reported that, for example, the amount of consumption increases in cancer cells, for glutamine mainly as an oxidation energy source, for arginine as a precursor of nitrogen oxide and polyamine, and for methionine through the activation of the ability of cancer cells to take in methionine, respectively. Vissers, et al. (“Vissers, Y. LJ., et. al., Plasma arginine concentration are reduced in cancer patients: evidence for arginine deficiency?, The American Journal of Clinical Nutrition, 2005 81, p. 1142-1146”) and Kubota (“Kubota, A., Meguid, M. M., and Hitch, D. C., Amino acid profiles correlate diagnostically with organ site in three kinds of malignant tumors., Cancer, 1991, 69, p 2343-2348”) have reported that the amino acid composition in plasma in gastric cancer patients is different from that of healthy individuals.

WO 2004/052191 Pamphlet and WO 2006/098192 Pamphlet disclose a method of associating amino acid concentration with biological state.

However, there is a problem that the development of techniques of diagnosing the presence or absence of onset of gastric cancer with a plurality of amino acids as explanatory variables is not conducted from the viewpoint of time and cost and is not practically used. In addition, there is a problem that when the presence or absence of onset of gastric cancer is evaluated by index formulae disclosed in WO 2004/052191 Pamphlet and WO 2006/098192 Pamphlet, sufficient precision cannot be obtained.

### SUMMARY OF THE INVENTION

It is an object of the present invention to at least partially solve the problems in the conventional technology. The present invention is made in view of the problem described above, and an object of the present invention is to provide a method of evaluating gastric cancer, a gastric cancer-evaluating apparatus, a gastric cancer-evaluating method, a gastric cancer-evaluating system, a gastric cancer-evaluating program and a recording medium, which are capable of evaluating a gastric cancer state accurately by utilizing the concentration of amino acids related to a gastric cancer state among amino acids in blood.

The present inventors have made extensive study for solving the problem described above, and as a result they have identified amino acids which are useful in discrimination of between 2 groups of gastric cancer and gastric cancer-free (specifically, amino acids varying with a statistically significant difference between the 2 groups of the gastric cancer and the gastric cancer-free), amino acids which are useful in discrimination of a stage of gastric cancer (specifically, amino acids varying with a statistically significant difference in stage Ia, Ib, II, IIIa, IIIb, and IV of gastric cancer), and amino acids which are useful in discrimination of the presence or absence of metastasis of gastric cancer to other organs (specifically, amino acids varying with a statistically significant difference between the 2 groups of the presence of metastasis and the absence of metastasis to other organs), and have found that multivariate discriminant (index formula, correlation equation) including the concentrations of the identified amino acids as explanatory variables correlates significantly with the state (specifically, progress of a morbid state) of gastric cancer (specifically, early gastric cancer), and the present invention is thereby completed.

To solve the problem and achieve the object described above, a method of evaluating gastric cancer according to one aspect of the present invention includes a measuring step of measuring amino acid concentration data on a concentration value of an amino acid in blood collected from a subject to be evaluated, and a concentration value criterion evaluating step of evaluating a gastric cancer state in the subject based on the concentration value of at least one of Asn, Cys, His, Met, Orn, Phe, Trp, Pro, Lys, Leu, Glu, Arg, Ala, Thr, and Tyr contained in the amino acid concentration data of the subject measured at the measuring step.

Another aspect of the present invention is the method of evaluating gastric cancer, wherein the concentration value criterion evaluating step further includes a concentration value criterion discriminating step of discriminating between gastric cancer and gastric cancer-free, discriminating a stage of gastric cancer, or discriminating the presence or absence of metastasis of gastric cancer to other organ in the subject based on the concentration value of at least one of Asn, Cys, His, Met, Orn, Phe, Trp, Pro, Lys, Leu, Glu, Arg, Ala, Thr, and Tyr contained in the amino acid concentration data of the subject measured at the measuring step.

Still another aspect of the present invention is the method of evaluating gastric cancer, wherein the concentration criterion evaluating step further includes a discriminant value calculating step of calculating a discriminant value that is a value of a multivariate discriminant with a concentration of the amino acid as an explanatory variable, based on both the concentration value of at least one of Asn, Cys, His, Met, Orn, Phe, Trp, Pro, Lys, Leu, Glu, Arg, Ala, Thr, and Tyr contained in the amino acid concentration data of the subject measured at the measuring step and the previously established multivariate discriminant, and a discriminant value criterion evaluating step of evaluating the gastric cancer state in the subject based on the discriminant value calculated at the discriminant value calculating step, wherein the multivariate discriminant contains at least one of Asn, Cys, His, Met, Orn, Phe, Trp, Pro, Lys, Leu, Glu, Arg, Ala, Thr, and Tyr as the explanatory variable.

Still another aspect of the present invention is the method of evaluating gastric cancer, wherein the discriminant value criterion evaluating step further includes a discriminant value criterion discriminating step of discriminating between gastric cancer and gastric cancer-free, discriminating a stage of gastric cancer, or discriminating the presence or absence of metastasis of gastric cancer to other organ in the subject based on the discriminant value calculated at the discriminant value calculating step.

Still another aspect of the present invention is the method of evaluating gastric cancer, wherein the multivariate discriminant is expressed by one fractional expression or the sum of a plurality of the fractional expressions and contains at least one of Asn, Cys, His, Met, Orn, Phe, Trp, Pro, Lys, Leu, Glu, Arg, Ala, Thr, and Tyr as the explanatory variable in any one of the numerator and denominator or both in the fractional expression constituting the multivariate discriminant.

Still another aspect of the present invention is the method of evaluating gastric cancer, wherein (a) when the discrimination between the gastric cancer and the gastric cancer-free is conducted at the discriminant value criterion discriminating step, the multivariate discriminant is formula 1, 2, or 3, (b) when the discrimination of the stage of gastric cancer is conducted at the discriminant value criterion discriminating step, the multivariate discriminant is formula 4, and (c) when the discrimination of the presence or absence of metastasis of gastric cancer to other organ is conducted at the discriminant value criterion discriminating step, the multivariate discriminant is formula 5:

a₁×Orn/(Trp+His)+b₁×(ABA+Ile)/Leu+c₁ (formula 1)

a₂×Glu/His+b₂×Ser/Trp+c₂×Arg/Pro+d₂ (formula 2)

a₃×Trp/Gln+b₃×His/Glu+c₃ (formula 3)

a₄×Gly/(Glu+Trp+Val)+b₄×Arg/His+c₄ (formula 4)

a₅×Ile/Glu+b₅×(Gly+Asn+Arg)/His+c₅ (formula 5)

wherein a₁ and b₁ in the formula 1 are arbitrary non-zero real numbers, c₁ in the formula 1 is arbitrary real number, a₂, b₂, and c₂ in the formula 2 are arbitrary non-zero real numbers, d₂ in the formula 2 is arbitrary real number, a₃ and b₃ in the formula 3 are arbitrary non-zero real numbers, c₃ in the formula 3 is arbitrary real number, a₄ and b₄ in the formula 4 are arbitrary non-zero real numbers, c₄ in the formula 4 is arbitrary real number, a₅ and b₅ in the formula 5 are arbitrary non-zero real numbers, and c₅ in the formula 5 is arbitrary real number.

Still another aspect of the present invention is the method of evaluating gastric cancer, wherein the multivariate discriminant is any one of a logistic regression equation, a linear discriminant, a multiple regression equation, a discriminant prepared by a support vector machine, a discriminant prepared by a Mahalanobis' generalized distance method, a discriminant prepared by canonical discriminant analysis, and a discriminant prepared by a decision tree.

Still another aspect of the present invention is the method of evaluating gastric cancer, wherein the multivariate discriminant is the logistic regression equation with Orn, Gln, Trp, and Cit as the explanatory variables, the linear discriminant with Orn, Gln, Trp, Phe, Cit, and Try as the explanatory variables, the logistic regression equation with Glu, Phe, His, and Trp as the explanatory variables, the linear discriminant with Glu, Pro, His, and Trp as the explanatory variables, the logistic regression equation with Val, Ile, His, and Trp as the explanatory variables, or the linear discriminant with Thr, Ile, His, and Trp as the explanatory variables.

The present invention also relates to a gastric cancer-evaluating apparatus, the gastric cancer-evaluating apparatus according to one aspect of the present invention includes a control unit and a memory unit to evaluate a gastric cancer state in a subject to be evaluated. The control unit includes a discriminant value-calculating unit that calculates a discriminant value that is a value of a multivariate discriminant with a concentration of an amino acid as an explanatory variable, based on both (a) a concentration value of at least one of Asn, Cys, His, Met, Orn, Phe, Trp, Pro, Lys, Leu, Glu, Arg, Ala, Thr, and Tyr contained in previously obtained amino acid concentration data of the subject on the concentration value of the amino acid and (b) the multivariate discriminant containing at least one of Asn, Cys, His, Met, Orn, Phe, Trp, Pro, Lys, Leu, Glu, Arg, Ala, Thr, and Tyr as the explanatory variable, stored in the memory unit, and a discriminant value criterion-evaluating unit that evaluates the gastric cancer state in the subject based on the discriminant value calculated by the discriminant value-calculating unit.

Another aspect of the present invention is the gastric cancer-evaluating apparatus, wherein the discriminant value criterion-evaluating unit further includes a discriminant value criterion-discriminating unit that discriminates between gastric cancer and gastric cancer-free, discriminates a stage of gastric cancer, or discriminates the presence or absence of metastasis of gastric cancer to other organ in the subject based on the discriminant value calculated by the discriminant value-calculating unit.

Still another aspect of the present invention is the gastric cancer-evaluating apparatus, wherein the multivariate discriminant is expressed by one fractional expression or the sum of a plurality of the fractional expressions and contains at least one of Asn, Cys, His, Met, Orn, Phe, Trp, Pro, Lys, Leu, Glu, Arg, Ala, Thr, and Tyr as the explanatory variable in any one of the numerator and denominator or both in the fractional expression constituting the multivariate discriminant.

Still another aspect of the present invention is the gastric cancer-evaluating apparatus, wherein (a) when the discrimination between the gastric cancer and the gastric cancer-free is conducted by the discriminant value criterion-discriminating unit, the multivariate discriminant is formula 1, 2, or 3, (b) when the discrimination of the stage of gastric cancer is conducted by the discriminant value criterion-discriminating unit, the multivariate discriminant is formula 4, and (c) when the discrimination of the presence or absence of metastasis of gastric cancer to other organ is conducted by the discriminant value criterion-discriminating unit, the multivariate discriminant is formula 5:

a₁×Orn/(Trp+His)+b₁×(ABA+Ile)/Leu+c₁ (formula 1)

a₂×Glu/His+b₂×Ser/Trp+c₂×Arg/Pro+d₂ (formula 2)

a₃×Trp/Gln+b₃×His/Glu+c₃ (formula 3)

a₄×Gly/(Glu+Trp+Val)+b₄×Arg/His+c₄ (formula 4)

a₅×Ile/Glu+b₅×(Gly+Asn+Arg)/His+c₅ (formula 5)

wherein a₁ and b₁ in the formula 1 are arbitrary non-zero real numbers, c₁ in the formula 1 is arbitrary real number, a₂, b₂, and c₂ in the formula 2 are arbitrary non-zero real numbers, d₂ in the formula 2 is arbitrary real number, a₃ and b₃ in the formula 3 are arbitrary non-zero real numbers, c₃ in the formula 3 is arbitrary real number, a₄ and b₄ in the formula 4 are arbitrary non-zero real numbers, c₄ in the formula 4 is arbitrary real number, a₅ and b₅ in the formula 5 are arbitrary non-zero real numbers, and c₅ in the formula 5 is arbitrary real number.

Still another aspect of the present invention is the gastric cancer-evaluating apparatus, wherein the multivariate discriminant is any one of a logistic regression equation, a linear discriminant, a multiple regression equation, a discriminant prepared by a support vector machine, a discriminant prepared by a Mahalanobis' generalized distance method, a discriminant prepared by canonical discriminant analysis, and a discriminant prepared by a decision tree.

Still another aspect of the present invention is the gastric cancer-evaluating apparatus, wherein the multivariate discriminant is the logistic regression equation with Orn, Gln, Trp, and Cit as the explanatory variables, the linear discriminant with Orn, Gln, Trp, Phe, Cit, and Try as the explanatory variables, the logistic regression equation with Glu, Phe, His, and Trp as the explanatory variables, the linear discriminant with Glu, Pro, His, and Trp as the explanatory variables, the logistic regression equation with Val, Ile, His, and Trp as the explanatory variables, or the linear discriminant with Thr, Ile, His, and Trp as the explanatory variables.

Still another aspect of the present invention is the gastric cancer-evaluating apparatus, wherein the control unit further includes a multivariate discriminant-preparing unit that prepares the multivariate discriminant stored in the memory unit, based on gastric cancer state information containing the amino acid concentration data and gastric cancer state index data on an index for indicating the gastric cancer state, stored in the memory unit. The multivariate discriminant-preparing unit further includes a candidate multivariate discriminant-preparing unit that prepares a candidate multivariate discriminant that is a candidate of the multivariate discriminant, based on a predetermined discriminant-preparing method from the gastric cancer state information, a candidate multivariate discriminant-verifying unit that verifies the candidate multivariate discriminant prepared by the candidate multivariate discriminant-preparing unit, based on a predetermined verifying method, and an explanatory variable-selecting unit that selects the explanatory variable of the candidate multivariate discriminant based on a predetermined explanatory variable-selecting method from a verification result obtained by the candidate multivariate discriminant-verifying unit, thereby selecting a combination of the amino acid concentration data contained in the gastric cancer state information used in preparing the candidate multivariate discriminant. The multivariate discriminant-preparing unit prepares the multivariate discriminant by selecting the candidate multivariate discriminant used as the multivariate discriminant, from a plurality of the candidate multivariate discriminants, based on the verification results accumulated by repeatedly executing the candidate multivariate discriminant-preparing unit, the candidate multivariate discriminant-verifying unit, and the explanatory variable-selecting unit.

The present invention also relates to a gastric cancer-evaluating method, one aspect of the present invention is the gastric cancer-evaluating method of evaluating a gastric cancer state in a subject to be evaluated. The method is carried out with an information processing apparatus including a control unit and a memory unit. The method includes (i) a discriminant value calculating step of calculating a discriminant value that is a value of a multivariate discriminant with a concentration of an amino acid as an explanatory variable, based on both (a) a concentration value of at least one of Asn, Cys, His, Met, Orn, Phe, Trp, Pro, Lys, Leu, Glu, Arg, Ala, Thr, and Tyr contained in previously obtained amino acid concentration data of the subject on the concentration value of the amino acid and (b) the multivariate discriminant containing at least one of Asn, Cys, His, Met, Orn, Phe, Trp, Pro, Lys, Leu, Glu, Arg, Ala, Thr, and Tyr as the explanatory variable, stored in the memory unit, and (ii) a discriminant value criterion evaluating step of evaluating the gastric cancer state in the subject based on the discriminant value calculated at the discriminant value calculating step. The steps (i) and (ii) are executed by the control unit.

Another aspect of the present invention is the gastric cancer-evaluating method, wherein the discriminant value criterion evaluating step further includes a discriminant value criterion discriminating step of discriminating between gastric cancer and gastric cancer-free, discriminating a stage of gastric cancer, or discriminating the presence or absence of metastasis of gastric cancer to other organ in the subject based on the discriminant value calculated at the discriminant value calculating step.

Still another aspect of the present invention is the gastric cancer-evaluating method, wherein the multivariate discriminant is expressed by one fractional expression or the sum of a plurality of the fractional expressions and contains at least one of Asn, Cys, His, Met, Orn, Phe, Trp, Pro, Lys, Leu, Glu, Arg, Ala, Thr, and Tyr as the explanatory variable in any one of the numerator and denominator or both in the fractional expression constituting the multivariate discriminant.

Still another aspect of the present invention is the gastric cancer-evaluating method, wherein (a) when the discrimination between the gastric cancer and the gastric cancer-free is conducted at the discriminant value criterion discriminating step, the multivariate discriminant is formula 1, 2, or 3, (b) when the discrimination of the stage of gastric cancer is conducted at the discriminant value criterion discriminating step, the multivariate discriminant is formula 4, and (c) when the discrimination of the presence or absence of metastasis of gastric cancer to other organ is conducted at the discriminant value criterion discriminating step, the multivariate discriminant is formula 5:

a₁×Orn/(Trp+His)+b₁×(ABA+Ile)/Leu+c₁ (formula 1)

a₂×Glu/His+b₂×Ser/Trp+c₂×Arg/Pro+d₂ (formula 2)

a₃×Trp/Gln+b₃×His/Glu+c₃ (formula 3)

a₄×Gly/(Glu+Trp+Val)+b₄×Arg/His+c₄ (formula 4)

a₅×Ile/Glu+b₅×(Gly+Asn+Arg)/His+c₅ (formula 5)

wherein a₁ and b₁ in the formula 1 are arbitrary non-zero real numbers, c₁ in the formula 1 is arbitrary real number, a₂, b₂, and c₂ in the formula 2 are arbitrary non-zero real numbers, d₂ in the formula 2 is arbitrary real number, a₃ and b₃ in the formula 3 are arbitrary non-zero real numbers, c₃ in the formula 3 is arbitrary real number, a₄ and b₄ in the formula 4 are arbitrary non-zero real numbers, c₄ in the formula 4 is arbitrary real number, a₅ and b₅ in the formula 5 are arbitrary non-zero real numbers, and c₅ in the formula 5 is arbitrary real number.

Still another aspect of the present invention is the gastric cancer-evaluating method, wherein the multivariate discriminant is any one of a logistic regression equation, a linear discriminant, a multiple regression equation, a discriminant prepared by a support vector machine, a discriminant prepared by a Mahalanobis' generalized distance method, a discriminant prepared by canonical discriminant analysis, and a discriminant prepared by a decision tree.

Still another aspect of the present invention is the gastric cancer-evaluating method, wherein the multivariate discriminant is the logistic regression equation with Orn, Gln, Trp, and Cit as the explanatory variables, the linear discriminant with Orn, Gln, Trp, Phe, Cit, and Try as the explanatory variables, the logistic regression equation with Glu, Phe, His, and Trp as the explanatory variables, the linear discriminant with Glu, Pro, His, and Trp as the explanatory variables, the logistic regression equation with Val, Ile, His, and Trp as the explanatory variables, or the linear discriminant with Thr, Ile, His, and Trp as the explanatory variables.

Still another aspect of the present invention is the gastric cancer-evaluating method, wherein the method further includes a multivariate discriminant preparing step of preparing the multivariate discriminant stored in the memory unit, based on gastric cancer state information containing the amino acid concentration data and gastric cancer state index date on an index for indicating the gastric cancer state, stored in the memory unit that is executed by the control unit. The multivariate discriminant preparing step further includes a candidate multivariate discriminant preparing step of preparing a candidate multivariate discriminant that is a candidate of the multivariate discriminant, based on a predetermined discriminant-preparing method from the gastric cancer state information, a candidate multivariate discriminant verifying step of verifying the candidate multivariate discriminant prepared at the candidate multivariate preparing step, based on a predetermined verifying method, and an explanatory variable selecting step of selecting the explanatory variable of the candidate multivariate discriminant based on a predetermined explanatory variable-selecting method from a verification result obtained at the candidate multivariate discriminant verifying step, thereby selecting a combination of the amino acid concentration data contained in the gastric cancer state information used in preparing the candidate multivariate discriminant. At the multivariate discriminant preparing step, the multivariate discriminant is prepared by selecting the candidate multivariate discriminant used as the multivariate discriminant from a plurality of the candidate multivariate discriminants, based on the verification results accumulated by repeatedly executing the candidate multivariate discriminant preparing step, the candidate multivariate discriminant verifying step, and the explanatory variable selecting step.

The present invention also relates to a gastric cancer-evaluating system, the gastric cancer-evaluating system according to one aspect of the present invention includes a gastric cancer-evaluating apparatus including a control unit and a memory unit to evaluate a gastric cancer state in a subject to be evaluated and an information communication terminal apparatus that provides amino acid concentration data of the subject on a concentration value of an amino acid connected to each other communicatively via a network. The information communication terminal apparatus includes an amino acid concentration data-sending unit that transmits the amino acid concentration data of the subject to the gastric cancer-evaluating apparatus, and an evaluation result-receiving unit that receives an evaluation result of the gastric cancer state of the subject transmitted from the gastric cancer-evaluating apparatus. The control unit of the gastric cancer-evaluating apparatus includes an amino acid concentration data-receiving unit that receives the amino acid concentration data of the subject transmitted from the information communication terminal apparatus, a discriminant value-calculating unit that calculates a discriminant value that is a value of a multivariate discriminant with a concentration of the amino acid as an explanatory variable, based on both (a) the concentration value of at least one of Asn, Cys, His, Met, Orn, Phe, Trp, Pro, Lys, Leu, Glu, Arg, Ala, Thr, and Tyr contained in the amino acid concentration data of the subject received by the amino acid concentration data-receiving unit and (b) the multivariate discriminant containing at least one of Asn, Cys, His, Met, Orn, Phe, Trp, Pro, Lys, Leu, Glu, Arg, Ala, Thr, and Tyr as the explanatory variable, stored in the memory unit, a discriminant value criterion-evaluating unit that evaluates the gastric cancer state in the subject based on the discriminant value calculated by the discriminant value-calculating unit, and an evaluation result-sending unit that transmits the evaluation result of the subject obtained by the discriminant value criterion-evaluating unit to the information communication terminal apparatus.

Another aspect of the present invention is the gastric cancer-evaluating system, wherein the discriminant value criterion-evaluating unit further includes a discriminant value criterion-discriminating unit that discriminates between gastric cancer and gastric cancer-free, discriminates a stage of gastric cancer, or discriminates the presence or absence of metastasis of gastric cancer to other organ in the subject based on the discriminant value calculated by the discriminant value-calculating unit.

Still another aspect of the present invention is the gastric cancer-evaluating system, wherein the multivariate discriminant is expressed by one fractional expression or the sum of a plurality of the fractional expressions and contains at least one of Asn, Cys, His, Met, Orn, Phe, Trp, Pro, Lys, Leu, Glu, Arg, Ala, Thr, and Tyr as the explanatory variable in any one of the numerator and denominator or both in the fractional expression constituting the multivariate discriminant.

Still another aspect of the present invention is the gastric cancer-evaluating system, wherein (a) when the discrimination between the gastric cancer and the gastric cancer-free is conducted by the discriminant value criterion-discriminating unit, the multivariate discriminant is formula 1, 2, or 3, (b) when the discrimination of the stage of gastric cancer is conducted by the discriminant value criterion-discriminating unit, the multivariate discriminant is formula 4, and (c) when the discrimination of the presence or absence of metastasis of gastric cancer to other organ is conducted by the discriminant value criterion-discriminating unit, the multivariate discriminant is formula 5:

a₁×Orn/(Trp+His)+b₁×(ABA+Ile)/Leu+c₁ (formula 1)

a₂×Glu/His+b₂×Ser/Trp+c₂×Arg/Pro+d₂ (formula 2)

a₃×Trp/Gln+b₃×His/Glu+c₃ (formula 3)

a₄×Gly/(Glu+Trp+Val)+b₄×Arg/His+c₄ (formula 4)

a₅×Ile/Glu+b₅×(Gly+Asn+Arg)/His+c₅ (formula 5)

wherein a₁ and b₁ in the formula 1 are arbitrary non-zero real numbers, c₁ in the formula 1 is arbitrary real number, a₂, b₂, and c₂ in the formula 2 are arbitrary non-zero real numbers, d₂ in the formula 2 is arbitrary real number, a₃ and b₃ in the formula 3 are arbitrary non-zero real numbers, c₃ in the formula 3 is arbitrary real number, a₄ and b₄ in the formula 4 are arbitrary non-zero real numbers, c₄ in the formula 4 is arbitrary real number, a₅ and b₅ in the formula 5 are arbitrary non-zero real numbers, and c₅ in the formula 5 is arbitrary real number.

Still another aspect of the present invention is the gastric cancer-evaluating system, wherein the multivariate discriminant is any one of a logistic regression equation, a linear discriminant, a multiple regression equation, a discriminant prepared by a support vector machine, a discriminant prepared by a Mahalanobis' generalized distance method, a discriminant prepared by canonical discriminant analysis, and a discriminant prepared by a decision tree.

Still another aspect of the present invention is the gastric cancer-evaluating system, wherein the multivariate discriminant is the logistic regression equation with Orn, Gln, Trp, and Cit as the explanatory variables, the linear discriminant with Orn, Gln, Trp, Phe, Cit, and Try as the explanatory variables, the logistic regression equation with Glu, Phe, His, and Trp as the explanatory variables, the linear discriminant with Glu, Pro, His, and Trp as the explanatory variables, the logistic regression equation with Val, Ile, His, and Trp as the explanatory variables, or the linear discriminant with Thr, Ile, His, and Trp as the explanatory variables.

Still another aspect of the present invention is the gastric cancer-evaluating system, wherein the control unit further includes a multivariate discriminant-preparing unit that prepares the multivariate discriminant stored in the memory unit, based on gastric cancer state information containing the amino acid concentration data and gastric cancer state index data on an index for indicating the gastric cancer state, stored in the memory unit. The multivariate discriminant-preparing unit further includes a candidate multivariate discriminant-preparing unit that prepares a candidate multivariate discriminant that is a candidate of the multivariate discriminant, based on a predetermined discriminant-preparing method from the gastric cancer state information, a candidate multivariate discriminant-verifying unit that verifies the candidate multivariate discriminant prepared by the candidate multivariate discriminant-preparing unit, based on a predetermined verifying method, and an explanatory variable-selecting unit that selects the explanatory variable of the candidate multivariate discriminant based on a predetermined explanatory variable-selecting method from a verification result obtained by the candidate multivariate discriminant-verifying unit, thereby selecting a combination of the amino acid concentration data contained in the gastric cancer state information used in preparing the candidate multivariate discriminant. The multivariate discriminant-preparing unit prepares the multivariate discriminant by selecting the candidate multivariate discriminant used as the multivariate discriminant, from a plurality of the candidate multivariate discriminants, based on the verification results accumulated by repeatedly executing the candidate multivariate discriminant-preparing unit, the candidate multivariate discriminant-verifying unit, and the explanatory variable-selecting unit.

The present invention also relates to a gastric cancer-evaluating program product, one aspect of the present invention is the gastric cancer-evaluating program product that makes an information processing apparatus including a control unit and a memory unit execute a method of evaluating a gastric cancer state in a subject to be evaluated. The method includes (i) a discriminant value calculating step of calculating a discriminant value that is a value of a multivariate discriminant with a concentration of an amino acid as an explanatory variable, based on both (a) a concentration value of at least one of Asn, Cys, His, Met, Orn, Phe, Trp, Pro, Lys, Leu, Glu, Arg, Ala, Thr, and Tyr contained in previously obtained amino acid concentration data of the subject on the concentration value of the amino acid and (b) the multivariate discriminant containing at least one of Asn, Cys, His, Met, Orn, Phe, Trp, Pro, Lys, Leu, Glu, Arg, Ala, Thr, and Tyr as the explanatory variable, stored in the memory unit, and (ii) a discriminant value criterion evaluating step of evaluating the gastric cancer state in the subject based on the discriminant value calculated at the discriminant value calculating step. The steps (i) and (ii) are executed by the control unit.

Another aspect of the present invention is the gastric cancer-evaluating program product, wherein the discriminant value criterion evaluating step further includes a discriminant value criterion discriminating step of discriminating between gastric cancer and gastric cancer-free, discriminating a stage of gastric cancer, or discriminating the presence or absence of metastasis of gastric cancer to other organ in the subject based on the discriminant value calculated at the discriminant value calculating step.

Still another aspect of the present invention is the gastric cancer-evaluating program product, wherein the multivariate discriminant is expressed by one fractional expression or the sum of a plurality of the fractional expressions and contains at least one of Asn, Cys, His, Met, Orn, Phe, Trp, Pro, Lys, Leu, Glu, Arg, Ala, Thr, and Tyr as the explanatory variable in any one of the numerator and denominator or both in the fractional expression constituting the multivariate discriminant.

Still another aspect of the present invention is the gastric cancer-evaluating program product, wherein (a) when the discrimination between the gastric cancer and the gastric cancer-free is conducted at the discriminant value criterion discriminating step, the multivariate discriminant is formula 1, 2, or 3, (b) when the discrimination of the stage of gastric cancer is conducted at the discriminant value criterion discriminating step, the multivariate discriminant is formula 4, and (c) when the discrimination of the presence or absence of metastasis of gastric cancer to other organ is conducted at the discriminant value criterion discriminating step, the multivariate discriminant is formula 5:

a₁×Orn/(Trp+His)+b₁×(ABA+Ile)/Leu+c₁ (formula 1)

a₂×Glu/His+b₂×Ser/Trp+c₂×Arg/Pro+d₂ (formula 2)

a₃×Trp/Gln+b₃×His/Glu+c₃ (formula 3)

a₄×Gly/(Glu+Trp+Val)+b₄×Arg/His+c₄ (formula 4)

a₅×Ile/Glu+b₅×(Gly+Asn+Arg)/His+c₅ (formula 5)

wherein a₁ and b₁ in the formula 1 are arbitrary non-zero real numbers, c₁ in the formula 1 is arbitrary real number, a₂, b₂, and c₂ in the formula 2 are arbitrary non-zero real numbers, d₂ in the formula 2 is arbitrary real number, a₃ and b₃ in the formula 3 are arbitrary non-zero real numbers, c₃ in the formula 3 is arbitrary real number, a₄ and b₄ in the formula 4 are arbitrary non-zero real numbers, c₄ in the formula 4 is arbitrary real number, a₅ and b₅ in the formula 5 are arbitrary non-zero real numbers, and c₅ in the formula 5 is arbitrary real number.

Still another aspect of the present invention is the gastric cancer-evaluating program product, wherein the multivariate discriminant is any one of a logistic regression equation, a linear discriminant, a multiple regression equation, a discriminant prepared by a support vector machine, a discriminant prepared by a Mahalanobis' generalized distance method, a discriminant prepared by canonical discriminant analysis, and a discriminant prepared by a decision tree.

Still another aspect of the present invention is the gastric cancer-evaluating program product, wherein the multivariate discriminant is the logistic regression equation with Orn, Gln, Trp, and Cit as the explanatory variables, the linear discriminant with Orn, Gln, Trp, Phe, Cit, and Try as the explanatory variables, the logistic regression equation with Glu, Phe, His, and Trp as the explanatory variables, the linear discriminant with Glu, Pro, His, and Trp as the explanatory variables, the logistic regression equation with Val, Ile, His, and Trp as the explanatory variables, or the linear discriminant with Thr, Ile, His, and Trp as the explanatory variables. Still another aspect of the present invention is the gastric cancer-evaluating program product, wherein the method further includes a multivariate discriminant preparing step of preparing the multivariate discriminant stored in the memory unit, based on gastric cancer state information containing the amino acid concentration data and gastric cancer state index date on an index for indicating the gastric cancer state, stored in the memory unit that is executed by the control unit. The multivariate discriminant preparing step further includes a candidate multivariate discriminant preparing step of preparing a candidate multivariate discriminant that is a candidate of the multivariate discriminant, based on a predetermined discriminant-preparing method from the gastric cancer state information, a candidate multivariate discriminant verifying step of verifying the candidate multivariate discriminant prepared at the candidate multivariate preparing step, based on a predetermined verifying method, and an explanatory variable selecting step of selecting the explanatory variable of the candidate multivariate discriminant based on a predetermined explanatory variable-selecting method from a verification result obtained at the candidate multivariate discriminant verifying step, thereby selecting a combination of the amino acid concentration data contained in the gastric cancer state information used in preparing the candidate multivariate discriminant. At the multivariate discriminant preparing step, the multivariate discriminant is prepared by selecting the candidate multivariate discriminant used as the multivariate discriminant from a plurality of the candidate multivariate discriminants, based on the verification results accumulated by repeatedly executing the candidate multivariate discriminant preparing step, the candidate multivariate discriminant verifying step, and the explanatory variable selecting step.

The present invention also relates to a recording medium, the recording medium according to one aspect of the present invention includes the gastric cancer-evaluating program product described above.

According to the method of evaluating gastric cancer of the present invention, amino acid concentration data on a concentration value of an amino acid in blood collected from a subject to be evaluated is measured, and a gastric cancer state in the subject is evaluated based on the concentration value of at least one of Asn, Cys, His, Met, Orn, Phe, Trp, Pro, Lys, Leu, Glu, Arg, Ala, Thr, and Tyr contained in the measured amino acid concentration data of the subject. Thus, concentrations of amino acids which among amino acids in blood, are related to the gastric cancer state can be utilized to bring about an effect of enabling an accurate evaluation of the gastric cancer state.

According to the method of evaluating gastric cancer of the present invention, discrimination between gastric cancer and gastric cancer-free, discrimination a stage of gastric cancer, or discrimination the presence or absence of metastasis of gastric cancer to other organ in the subject is conducted based on the concentration value of at least one of Asn, Cys, His, Met, Orn, Phe, Trp, Pro, Lys, Leu, Glu, Arg, Ala, Thr, and Tyr contained in the measured amino acid concentration data of the subject. Thus, concentrations of amino acids which among amino acids in blood, are useful for discriminating between the 2 groups of the gastric cancer and the gastric cancer-free, discriminating the stage of gastric cancer, or discriminating between the 2 groups of the presence of metastasis of gastric cancer to other organ and the absence of the metastasis can be utilized to bring about an effect of enabling accurately these discriminations.

According to the method of evaluating gastric cancer of the present invention, a discriminant value that is a value of a multivariate discriminant with a concentration of the amino acid as an explanatory variable is calculated based on both (a) the concentration value of at least one of Asn, Cys, His, Met, Orn, Phe, Trp, Pro, Lys, Leu, Glu, Arg, Ala, Thr, and Tyr contained in the measured amino acid concentration data of the subject and (b) the previously established multivariate discriminant containing at least one of Asn, Cys, His, Met, Orn, Phe, Trp, Pro, Lys, Leu, Glu, Arg, Ala, Thr, and Tyr as the explanatory variable, and the gastric cancer state in the subject is evaluated based on the calculated discriminant value. Thus, discriminant values obtained in multivariate discriminants which are correlated significantly with the gastric cancer state can be utilized to bring about an effect of enabling an accurate evaluation of the gastric cancer state.

According to the method of evaluating gastric cancer of the present invention, discrimination between gastric cancer and gastric cancer-free, discrimination a stage of gastric cancer, or discrimination the presence or absence of metastasis of gastric cancer to other organ in the subject is conducted based on the calculated discriminant value. Thus, discriminant values obtained in multivariate discriminants useful for discriminating between the 2 groups of the gastric cancer and the gastric cancer-free, discriminating the stage of gastric cancer, or discriminating between the 2 groups of the presence of metastasis of gastric cancer to other organ and the absence of the metastasis can be utilized to bring about an effect of enabling accurately these discriminations.

According to the method of evaluating gastric cancer of the present invention, the multivariate discriminant is expressed by one fractional expression or the sum of a plurality of the fractional expressions and contains at least one of Asn, Cys, His, Met, Orn, Phe, Trp, Pro, Lys, Leu, Glu, Arg, Ala, Thr, and Tyr as the explanatory variable in any one of the numerator and denominator or both in the fractional expression constituting the multivariate discriminant. Thus, discriminant values obtained in multivariate discriminants useful particularly for discriminating between the 2 groups of the gastric cancer and the gastric cancer-free, discriminating the stage of gastric cancer, or discriminating between the 2 groups of the presence of metastasis of gastric cancer to other organ and the absence of the metastasis can be utilized to bring about an effect of enabling more accurately these discriminations.

According to the method of evaluating gastric cancer of the present invention, (a) when the discrimination between the gastric cancer and the gastric cancer-free is conducted, the multivariate discriminant is formula 1, 2, or 3, (b) when the discrimination of the stage of gastric cancer is conducted, the multivariate discriminant is formula 4, and (c) when the discrimination of the presence or absence of metastasis of gastric cancer to other organ is conducted, the multivariate discriminant is formula 5:

a₁×Orn/(Trp+His)+b₁×(ABA+Ile)/Leu+c₁ (formula 1)

a₂×Glu/His+b₂×Ser/Trp+c₂×Arg/Pro+d₂ (formula 2)

a₃×Trp/Gln+b₃×His/Glu+c₃ (formula 3)

a₄×Gly/(Glu+Trp+Val)+b₄×Arg/His+c₄ (formula 4)

a₅×Ile/Glu+b₅×(Gly+Asn+Arg)/His+c₅ (formula 5)

wherein a₁ and b₁ in the formula 1 are arbitrary non-zero real numbers, c₁ in the formula 1 is arbitrary real number, a₂, b₂, and c₂ in the formula 2 are arbitrary non-zero real numbers, d₂ in the formula 2 is arbitrary real number, a₃ and b₃ in the formula 3 are arbitrary non-zero real numbers, c₃ in the formula 3 is arbitrary real number, a₄ and b₄ in the formula 4 are arbitrary non-zero real numbers, c₄ in the formula 4 is arbitrary real number, a₅ and b₅ in the formula 5 are arbitrary non-zero real numbers, and c₅ in the formula 5 is arbitrary real number. Thus, discriminant values obtained in multivariate discriminants useful particularly for discriminating between the 2 groups of the gastric cancer and the gastric cancer-free, discriminating the stage of gastric cancer, or discriminating between the 2 groups of the presence of metastasis of gastric cancer to other organ and the absence of the metastasis can be utilized to bring about an effect of enabling more accurately these discriminations.

According to the method of evaluating gastric cancer of the present invention, the multivariate discriminant is any one of a logistic regression equation, a linear discriminant, a multiple regression equation, a discriminant prepared by a support vector machine, a discriminant prepared by a Mahalanobis' generalized distance method, a discriminant prepared by canonical discriminant analysis, and a discriminant prepared by a decision tree. Thus, discriminant values obtained in multivariate discriminants useful particularly for discriminating between the 2 groups of the gastric cancer and the gastric cancer-free, discriminating the stage of gastric cancer, or discriminating between the 2 groups of the presence of metastasis of gastric cancer to other organ and the absence of the metastasis can be utilized to bring about an effect of enabling more accurately these discriminations.

According to the method of evaluating gastric cancer of the present invention, the multivariate discriminant is the logistic regression equation with Orn, Gln, Trp, and Cit as the explanatory variables, the linear discriminant with Orn, Gln, Trp, Phe, Cit, and Try as the explanatory variables, the logistic regression equation with Glu, Phe, His, and Trp as the explanatory variables, the linear discriminant with Glu, Pro, His, and Trp as the explanatory variables, the logistic regression equation with Val, Ile, His, and Trp as the explanatory variables, or the linear discriminant with Thr, Ile, His, and Trp as the explanatory variables. Thus, discriminant values obtained in multivariate discriminants useful particularly for discriminating between the 2 groups of the gastric cancer and the gastric cancer-free, discriminating the stage of gastric cancer, or discriminating between the 2 groups of the presence of metastasis of gastric cancer to other organ and the absence of the metastasis can be utilized to bring about an effect of enabling more accurately these discriminations.

According to the gastric cancer-evaluating apparatus, the gastric cancer-evaluating method and the gastric cancer-evaluating program of the present invention, a discriminant value that is a value of a multivariate discriminant with a concentration of an amino acid as an explanatory variable is calculated based on both (a) a concentration value of at least one of Asn, Cys, His, Met, Orn, Phe, Trp, Pro, Lys, Leu, Glu, Arg, Ala, Thr, and Tyr contained in previously obtained amino acid concentration data of a subject to be evaluated on the concentration value of the amino acid and (b) the multivariate discriminant containing at least one of Asn, Cys, His, Met, Orn, Phe, Trp, Pro, Lys, Leu, Glu, Arg, Ala, Thr, and Tyr as the explanatory variable, stored in a memory unit, and a gastric cancer state in the subject is evaluated based on the calculated discriminant value. Thus, discriminant values obtained in multivariate discriminants which are correlated significantly with the gastric cancer state can be utilized to bring about an effect of enabling an accurate evaluation of the gastric cancer state.

According to the gastric cancer-evaluating apparatus, the gastric cancer-evaluating method and the gastric cancer-evaluating program of the present invention, discrimination between gastric cancer and gastric cancer-free, discrimination a stage of gastric cancer, or discrimination the presence or absence of metastasis of gastric cancer to other organ in the subject is conducted based on the calculated discriminant value. Thus, discriminant values obtained in multivariate discriminants useful for discriminating between the 2 groups of the gastric cancer and the gastric cancer-free, discriminating the stage of gastric cancer, or discriminating between the 2 groups of the presence of metastasis of gastric cancer to other organ and the absence of the metastasis can be utilized to bring about an effect of enabling accurately these discriminations.

According to the gastric cancer-evaluating apparatus, the gastric cancer-evaluating method and the gastric cancer-evaluating program of the present invention, the multivariate discriminant is expressed by one fractional expression or the sum of a plurality of the fractional expressions and contains at least one of Asn, Cys, His, Met, Orn, Phe, Trp, Pro, Lys, Leu, Glu, Arg, Ala, Thr, and Tyr as the explanatory variable in any one of the numerator and denominator or both in the fractional expression constituting the multivariate discriminant. Thus, discriminant values obtained in multivariate discriminants useful particularly for discriminating between the 2 groups of the gastric cancer and the gastric cancer-free, discriminating the stage of gastric cancer, or discriminating between the 2 groups of the presence of metastasis of gastric cancer to other organ and the absence of the metastasis can be utilized to bring about an effect of enabling more accurately these discriminations.

According to the gastric cancer-evaluating apparatus, the gastric cancer-evaluating method and the gastric cancer-evaluating program of the present invention, (a) when the discrimination between the gastric cancer and the gastric cancer-free is conducted, the multivariate discriminant is formula 1, 2, or 3, (b) when the discrimination of the stage of gastric cancer is conducted, the multivariate discriminant is formula 4, and (c) when the discrimination of the presence or absence of metastasis of gastric cancer to other organ is conducted, the multivariate discriminant is formula 5:

a₁×Orn/(Trp+His)+b₁×(ABA+Ile)/Leu+c₁ (formula 1)

a₂×Glu/His+b₂×Ser/Trp+c₂×Arg/Pro+d₂ (formula 2)

a₃×Trp/Gln+b₃×His/Glu+c₃ (formula 3)

a₄×Gly/(Glu+Trp+Val)+b₄×Arg/His+c₄ (formula 4)

a₅×Ile/Glu+b₅×(Gly+Asn+Arg)/His+c₅ (formula 5)

wherein a₁ and b₁ in the formula 1 are arbitrary non-zero real numbers, c₁ in the formula 1 is arbitrary real number, a₂, b₂, and c₂ in the formula 2 are arbitrary non-zero real numbers, d₂ in the formula 2 is arbitrary real number, a₃ and b₃ in the formula 3 are arbitrary non-zero real numbers, c₃ in the formula 3 is arbitrary real number, a₄ and b₄ in the formula 4 are arbitrary non-zero real numbers, c₄ in the formula 4 is arbitrary real number, a₅ and b₅ in the formula 5 are arbitrary non-zero real numbers, and c₅ in the formula 5 is arbitrary real number. Thus, discriminant values obtained in multivariate discriminants useful particularly for discriminating between the 2 groups of the gastric cancer and the gastric cancer-free, discriminating the stage of gastric cancer, or discriminating between the 2 groups of the presence of metastasis of gastric cancer to other organ and the absence of the metastasis can be utilized to bring about an effect of enabling more accurately these discriminations.

According to the gastric cancer-evaluating apparatus, the gastric cancer-evaluating method and the gastric cancer-evaluating program of the present invention, the multivariate discriminant is any one of a logistic regression equation, a linear discriminant, a multiple regression equation, a discriminant prepared by a support vector machine, a discriminant prepared by a Mahalanobis' generalized distance method, a discriminant prepared by canonical discriminant analysis, and a discriminant prepared by a decision tree. Thus, discriminant values obtained in multivariate discriminants useful particularly for discriminating between the 2 groups of the gastric cancer and the gastric cancer-free, discriminating the stage of gastric cancer, or discriminating between the 2 groups of the presence of metastasis of gastric cancer to other organ and the absence of the metastasis can be utilized to bring about an effect of enabling more accurately these discriminations.

According to the gastric cancer-evaluating apparatus, the gastric cancer-evaluating method and the gastric cancer-evaluating program of the present invention, the multivariate discriminant is the logistic regression equation with Orn, Gln, Trp, and Cit as the explanatory variables, the linear discriminant with Orn, Gln, Trp, Phe, Cit, and Try as the explanatory variables, the logistic regression equation with Glu, Phe, His, and Trp as the explanatory variables, the linear discriminant with Glu, Pro, His, and Trp as the explanatory variables, the logistic regression equation with Val, Ile, His, and Trp as the explanatory variables, or the linear discriminant with Thr, Ile, His, and Trp as the explanatory variables. Thus, discriminant values obtained in multivariate discriminants useful particularly for discriminating between the 2 groups of the gastric cancer and the gastric cancer-free, discriminating the stage of gastric cancer, or discriminating between the 2 groups of the presence of metastasis of gastric cancer to other organ and the absence of the metastasis can be utilized to bring about an effect of enabling more accurately these discriminations.

According to the gastric cancer-evaluating apparatus, the gastric cancer-evaluating method and the gastric cancer-evaluating program of the present invention, the multivariate discriminant stored in the memory unit is prepared based on gastric cancer state information containing the amino acid concentration data and gastric cancer state index data on an index for indicating the gastric cancer state, stored in the memory unit. Specifically, (1) a candidate multivariate discriminant is prepared based on a predetermined discriminant-preparing method from the gastric cancer state information, (2) the prepared candidate multivariate discriminant is verified based on a predetermined verifying method, (3) the explanatory variables of the candidate multivariate discriminant are selected based on a predetermined explanatory variable-selecting method from verification results obtained by executing (2), thereby selecting a combination of the amino acid concentration data contained in the gastric cancer state information used in preparing the candidate multivariate discriminant, and (4) the candidate multivariate discriminant used as the multivariate discriminant is selected from a plurality of the candidate multivariate discriminants based on the verification results accumulated by repeatedly executing (1), (2) and (3), thereby preparing the multivariate discriminant. There can thereby be brought about an effect of enabling a preparation of multivariate discriminants most appropriate for evaluating the gastric cancer state (specifically, multivariate discriminants correlating significantly with the state (progress of a morbid state) of gastric cancer (early gastric cancer) (more specifically, multivariate discriminants useful for discriminating between the 2 groups of the gastric cancer and the gastric cancer-free, multivariate discriminants useful for discriminating the stage of gastric cancer, or multivariate discriminants useful for discriminating between the 2 groups of the presence of metastasis of gastric cancer to other organ and the absence of the metastasis)).

According to the gastric cancer-evaluating system of the present invention, an information communication terminal apparatus first transmits amino acid concentration data of a subject to be evaluated to a gastric cancer-evaluating apparatus. The gastric cancer-evaluating apparatus (i) receives the amino acid concentration data of the subject transmitted from the information communication terminal apparatus, (ii) calculates a discriminant value that is a value of a multivariate discriminant with a concentration of an amino acid as an explanatory variable, based on both (a) a concentration value of at least one of Asn, Cys, His, Met, Orn, Phe, Trp, Pro, Lys, Leu, Glu, Arg, Ala, Thr, and Tyr contained in the received amino acid concentration data of the subject and (b) the multivariate discriminant containing at least one of Asn, Cys, His, Met, Orn, Phe, Trp, Pro, Lys, Leu, Glu, Arg, Ala, Thr, and Tyr as the explanatory variable, stored in a memory unit, (iii) evaluates a gastric cancer state in the subject based on the calculated discriminant value, and (iv) transmits evaluation result of the subject to the information communication terminal apparatus. Then, the information communication terminal apparatus receives the evaluation result of the subject concerning the gastric cancer state transmitted from the gastric cancer-evaluating apparatus. Thus, discriminant values obtained in multivariate discriminants which are correlated significantly with the gastric cancer state can be utilized to bring about an effect of enabling an accurate evaluation of the gastric cancer state.

According to the gastric cancer-evaluating system of the present invention, discrimination between gastric cancer and gastric cancer-free, discrimination a stage of gastric cancer, or discrimination the presence or absence of metastasis of gastric cancer to other organ in the subject is conducted based on the calculated discriminant value. Thus, discriminant values obtained in multivariate discriminants useful for discriminating between the 2 groups of the gastric cancer and the gastric cancer-free, discriminating the stage of gastric cancer, or discriminating between the 2 groups of the presence of metastasis of gastric cancer to other organ and the absence of the metastasis can be utilized to bring about an effect of enabling accurately these discriminations.

According to the gastric cancer-evaluating system of the present invention, the multivariate discriminant is expressed by one fractional expression or the sum of a plurality of the fractional expressions and contains at least one of Asn, Cys, His, Met, Orn, Phe, Trp, Pro, Lys, Leu, Glu, Arg, Ala, Thr, and Tyr as the explanatory variable in any one of the numerator and denominator or both in the fractional expression constituting the multivariate discriminant. Thus, discriminant values obtained in multivariate discriminants useful particularly for discriminating between the 2 groups of the gastric cancer and the gastric cancer-free, discriminating the stage of gastric cancer, or discriminating between the 2 groups of the presence of metastasis of gastric cancer to other organ and the absence of the metastasis can be utilized to bring about an effect of enabling more accurately these discriminations.

According to the gastric cancer-evaluating system of the present invention, (a) when the discrimination between the gastric cancer and the gastric cancer-free is conducted, the multivariate discriminant is formula 1, 2, or 3, (b) when the discrimination of the stage of gastric cancer is conducted, the multivariate discriminant is formula 4, and (c) when the discrimination of the presence or absence of metastasis of gastric cancer to other organ is conducted, the multivariate discriminant is formula 5:

a₁×Orn/(Trp+His)+b₁×(ABA+Ile)/Leu+c₁ (formula 1)

a₂×Glu/His+b₂×Ser/Trp+c₂×Arg/Pro+d₂ (formula 2)

a₃×Trp/Gln+b₃×His/Glu+c₃ (formula 3)

a₄×Gly/(Glu+Trp+Val)+b₄×Arg/His+c₄ (formula 4)

a₅×Ile/Glu+b₅×(Gly+Asn+Arg)/His+c₅ (formula 5)

wherein a₁ and b₁ in the formula 1 are arbitrary non-zero real numbers, c₁ in the formula 1 is arbitrary real number, a₂, b₂, and c₂ in the formula 2 are arbitrary non-zero real numbers, d₂ in the formula 2 is arbitrary real number, a₃ and b₃ in the formula 3 are arbitrary non-zero real numbers, c₃ in the formula 3 is arbitrary real number, a₄ and b₄ in the formula 4 are arbitrary non-zero real numbers, c₄ in the formula 4 is arbitrary real number, a₅ and b₅ in the formula 5 are arbitrary non-zero real numbers, and c₅ in the formula 5 is arbitrary real number. Thus, discriminant values obtained in multivariate discriminants useful particularly for discriminating between the 2 groups of the gastric cancer and the gastric cancer-free, discriminating the stage of gastric cancer, or discriminating between the 2 groups of the presence of metastasis of gastric cancer to other organ and the absence of the metastasis can be utilized to bring about an effect of enabling more accurately these discriminations.

According to the gastric cancer-evaluating system of the present invention, the multivariate discriminant is any one of a logistic regression equation, a linear discriminant, a multiple regression equation, a discriminant prepared by a support vector machine, a discriminant prepared by a Mahalanobis' generalized distance method, a discriminant prepared by canonical discriminant analysis, and a discriminant prepared by a decision tree. Thus, discriminant values obtained in multivariate discriminants useful particularly for discriminating between the 2 groups of the gastric cancer and the gastric cancer-free, discriminating the stage of gastric cancer, or discriminating between the 2 groups of the presence of metastasis of gastric cancer to other organ and the absence of the metastasis can be utilized to bring about an effect of enabling more accurately these discriminations.

According to the gastric cancer-evaluating system of the present invention, the multivariate discriminant is the logistic regression equation with Orn, Gln, Trp, and Cit as the explanatory variables, the linear discriminant with Orn, Gln, Trp, Phe, Cit, and Try as the explanatory variables, the logistic regression equation with Glu, Phe, His, and Trp as the explanatory variables, the linear discriminant with Glu, Pro, His, and Trp as the explanatory variables, the logistic regression equation with Val, Ile, His, and Trp as the explanatory variables, or the linear discriminant with Thr, Ile, His, and Trp as the explanatory variables. Thus, discriminant values obtained in multivariate discriminants useful particularly for discriminating between the 2 groups of the gastric cancer and the gastric cancer-free, discriminating the stage of gastric cancer, or discriminating between the 2 groups of the presence of metastasis of gastric cancer to other organ and the absence of the metastasis can be utilized to bring about an effect of enabling more accurately these discriminations.

According to the gastric cancer-evaluating system of the present invention, the gastric cancer-evaluating apparatus prepares the multivariate discriminant stored in the memory unit based on gastric cancer state information containing the amino acid concentration data and gastric cancer state index data on an index for indicating the gastric cancer state, stored in the memory unit. Specifically, (1) a candidate multivariate discriminant is prepared based on a predetermined discriminant-preparing method from the gastric cancer state information, (2) the prepared candidate multivariate discriminant is verified based on a predetermined verifying method, (3) the explanatory variables of the candidate multivariate discriminant are selected based on a predetermined explanatory variable-selecting method from verification results obtained by executing (2), thereby selecting a combination of the amino acid concentration data contained in the gastric cancer state information used in preparing the candidate multivariate discriminant, and (4) the candidate multivariate discriminant used as the multivariate discriminant is selected from a plurality of the candidate multivariate discriminants based on the verification results accumulated by repeatedly executing (1), (2) and (3), thereby preparing the multivariate discriminant. There can thereby be brought about an effect of enabling a preparation of multivariate discriminants most appropriate for evaluating the gastric cancer state (specifically, multivariate discriminants correlating significantly with the state (progress of a morbid state) of gastric cancer (early gastric cancer) (more specifically, multivariate discriminants useful for discriminating between the 2 groups of the gastric cancer and the gastric cancer-free, multivariate discriminants useful for discriminating the stage of gastric cancer, or multivariate discriminants useful for discriminating between the 2 groups of the presence of metastasis of gastric cancer to other organ and the absence of the metastasis)).

According to the recording medium of the present invention, the gastric cancer-evaluating program recorded on the recording medium is read and executed by the computer, thereby allowing the computer to execute the gastric cancer-evaluating program, thus bringing about an effect of obtaining the same effect as in the gastric cancer-evaluating program.

When the gastric cancer state is evaluated (specifically, for example, the discrimination between the gastric cancer and the gastric cancer-free is conducted, the discrimination of the stage of gastric cancer is conducted, or the discrimination of the presence or absence of metastasis of gastric cancer to other organs is conducted) in the present invention, concentrations of other metabolites (biological metabolites), protein expression level, age and sex of the subject, biological indices or the like may be used in addition to the concentrations of the amino acids. When the gastric cancer state is evaluated (specifically, for example, the discrimination between the gastric cancer and the gastric cancer-free is conducted, the discrimination of the stage of gastric cancer is conducted, or the discrimination of the presence or absence of metastasis of gastric cancer to other organs is conducted) in the present invention, concentrations of other metabolites (biological metabolites), protein expression level, age and sex of the subject, biological indices or the like may be used as the explanatory variables in the multivariate discriminants in addition to the concentrations of the amino acids.

The above and other objects, features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a principle configurational diagram showing a basic principle of the present invention;

FIG. 2 is a flowchart showing one example of a method of evaluating gastric cancer according to a first embodiment;

FIG. 3 is a principle configurational diagram showing a basic principle of the present invention;

FIG. 4 is a diagram showing an example of an entire configuration of a present system;

FIG. 5 is a diagram showing another example of an entire configuration of the present system;

FIG. 6 is a block diagram showing an example of a configuration of a gastric cancer-evaluating apparatus 100 in the present system;

FIG. 7 is a chart showing an example of information stored in a user information file 106a;

FIG. 8 is a chart showing an example of information stored in an amino acid concentration data file 106b;

FIG. 9 is a chart showing an example of information stored in a gastric cancer state information file 106c;

FIG. 10 is a chart showing an example of information stored in a designated gastric cancer state information file 106d;

FIG. 11 is a chart showing an example of information stored in a candidate multivariable discriminant file 106e1;

FIG. 12 is a chart showing an example of information stored in a verification result file 106e2;

FIG. 13 is a chart showing an example of information stored in a selected gastric cancer state information file 106e3;

FIG. 14 is a chart showing an example of information stored in a multivariable discriminant file 106e4;

FIG. 15 is a chart showing an example of information stored in a discriminant value file 106f;

FIG. 16 is a chart showing an example of information stored in an evaluation result file 106g;

FIG. 17 is a block diagram showing a configuration of a multivariable discriminant-preparing part 102h;

FIG. 18 is a block diagram showing a configuration of a discriminant criterion-evaluating part 102j;

FIG. 19 is a block diagram showing an example of a configuration of a client apparatus 200 in the present system;

FIG. 20 is a block diagram showing an example of a configuration of a database apparatus 400 in the present system;

FIG. 21 is a flowchart showing an example of a gastric cancer evaluation service processing performed in the present system;

FIG. 22 is a flowchart showing an example of a multivariate discriminant-preparing processing performed in the gastric cancer-evaluating apparatus 100 in the present system;

FIG. 23 is boxplots showing distributions of amino acid explanatory variables between 2 groups of gastric cancer and gastric cancer-free;

FIG. 24 is a graph showing AUCs of ROC curves of amino acid explanatory variables;

FIG. 25 is a graph showing an ROC curve for evaluation of diagnostic performance between 2 groups;

FIG. 26 is a chart showing a list of indices having the same diagnostic performance as that of index formula 1;

FIG. 27 is a chart showing a list of indices having the same diagnostic performance as that of index formula 1;

FIG. 28 is a chart showing a list of indices having the same diagnostic performance as that of index formula 1;

FIG. 29 is a chart showing a list of indices having the same diagnostic performance as that of index formula 1;

FIG. 30 is a graph showing an ROC curve for evaluation of diagnostic performance between 2 groups;

FIG. 31 is a chart showing a list of indices having the same diagnostic performance as that of index formula 2;

FIG. 32 is a chart showing a list of indices having the same diagnostic performance as that of index formula 2;

FIG. 33 is a chart showing a list of indices having the same diagnostic performance as that of index formula 2;

FIG. 34 is a chart showing a list of indices having the same diagnostic performance as that of index formula 2;

FIG. 35 is a graph showing an ROC curve for evaluation of diagnostic performance between 2 groups;

FIG. 36 is a chart showing a list of indices having the same diagnostic performance as that of index formula 3;

FIG. 37 is a chart showing a list of indices having the same diagnostic performance as that of index formula 3;

FIG. 38 is a chart showing a list of indices having the same diagnostic performance as that of index formula 3;

FIG. 39 is a chart showing a list of indices having the same diagnostic performance as that of index formula 3;

FIG. 40 is a graph showing pathological stages of gastric cancer and values of index formula 4;

FIG. 41 is a chart showing a list of indices having the same diagnostic performance as that of index formula 4;

FIG. 42 is a chart showing a list of indices having the same diagnostic performance as that of index formula 4;

FIG. 43 is a chart showing a list of indices having the same diagnostic performance as that of index formula 4;

FIG. 44 is a chart showing a list of indices having the same diagnostic performance as that of index formula 4;

FIG. 45 is a graph showing pathological stages of gastric cancer and values of index formula 5;

FIG. 46 is a chart showing a list of indices having the same diagnostic performance as that of index formula 5;

FIG. 47 is a chart showing a list of indices having the same diagnostic performance as that of index formula 5;

FIG. 48 is a chart showing a list of indices having the same diagnostic performance as that of index formula 5;

FIG. 49 is a chart showing a list of indices having the same diagnostic performance as that of index formula 5;

FIG. 50 is a graph showing an ROC curve for evaluation of diagnostic performance between 2 groups;

FIG. 51 is a chart showing a list of indices having the same diagnostic performance as that of index formula 6;

FIG. 52 is a chart showing a list of indices having the same diagnostic performance as that of index formula 6;

FIG. 53 is a chart showing a list of indices having the same diagnostic performance as that of index formula 6;

FIG. 54 is a chart showing a list of indices having the same diagnostic performance as that of index formula 6;

FIG. 55 is a graph showing an ROC curve for evaluation of diagnostic performance between 2 groups;

FIG. 56 is a chart showing a list of indices having the same diagnostic performance as that of index formula 7;

FIG. 57 is a chart showing a list of indices having the same diagnostic performance as that of index formula 7;

FIG. 58 is a chart showing a list of indices having the same diagnostic performance as that of index formula 7;

FIG. 59 is a chart showing a list of indices having the same diagnostic performance as that of index formula 7;

FIG. 60 is a graph showing an ROC curve for evaluation of diagnostic performance between 2 groups;

FIG. 61 is a chart showing a list of indices having the same diagnostic performance as that of index formula 8;

FIG. 62 is a chart showing a list of indices having the same diagnostic performance as that of index formula 8;

FIG. 63 is a chart showing a list of indices having the same diagnostic performance as that of index formula 8;

FIG. 64 is a chart showing a list of indices having the same diagnostic performance as that of index formula 8;

FIG. 65 is a chart showing a list of amino acids extracted based on AUCs of ROC curves;

FIG. 66 is graphs showing distributions of amino acid explanatory variables between 2 groups of gastric cancer patients and gastric cancer-free subjects;

FIG. 67 is a graph showing AUCs of ROC curves of amino acid explanatory variables;

FIG. 68 is a graph showing an ROC curve for evaluation of diagnostic performance between 2 groups;

FIG. 69 is a chart showing a list of indices having the same diagnostic performance as that of index formula 9;

FIG. 70 is a chart showing a list of indices having the same diagnostic performance as that of index formula 9;

FIG. 71 is a graph showing an ROC curve for evaluation of diagnostic performance between 2 groups;

FIG. 72 is a chart showing a list of indices having the same diagnostic performance as that of index formula 10;

FIG. 73 is a chart showing a list of indices having the same diagnostic performance as that of index formula 10;

FIG. 74 is a graph showing an ROC curve for evaluation of diagnostic performance between 2 groups;

FIG. 75 is a chart showing a list of indices having the same diagnostic performance as that of index formula 11;

FIG. 76 is a chart showing a list of indices having the same diagnostic performance as that of index formula 11;

FIG. 77 is a chart showing a list of amino acids extracted based on AUCs of ROC curves;

FIG. 78 is graphs showing distributions of amino acid explanatory variables between 2 groups of gastric cancer patients and gastric cancer-free subjects;

FIG. 79 is a graph showing AUCs of ROC curves of amino acid explanatory variables;

FIG. 80 is a chart showing a list of indices having the same diagnostic performance as that of index formula 12;

FIG. 81 is a chart showing a list of indices having the same diagnostic performance as that of index formula 12;

FIG. 82 is a chart showing a list of indices having the same diagnostic performance as that of index formula 12;

FIG. 83 is a chart showing a list of indices having the same diagnostic performance as that of index formula 12;

FIG. 84 is a graph showing an ROC curve for evaluation of diagnostic performance between 2 groups;

FIG. 85 is a chart showing a list of indices having the same diagnostic performance as that of index formula 13;

FIG. 86 is a chart showing a list of indices having the same diagnostic performance as that of index formula 13;

FIG. 87 is a chart showing a list of indices having the same diagnostic performance as that of index formula 13;

FIG. 88 is a chart showing a list of indices having the same diagnostic performance as that of index formula 13;

FIG. 89 is a graph showing an ROC curve for evaluation of diagnostic performance between 2 groups;

FIG. 90 is a chart showing a list of indices having the same diagnostic performance as that of index formula 14;

FIG. 91 is a chart showing a list of indices having the same diagnostic performance as that of index formula 14;

FIG. 92 is a chart showing a list of indices having the same diagnostic performance as that of index formula 14;

FIG. 93 is a graph showing an ROC curve for evaluation of diagnostic performance between 2 groups; and

FIG. 94 is a chart showing a list of amino acids extracted based on AUCs of ROC curves.

### DETAILED DESCRIPTION OF THE PREFERRED EMBODIMENTS

Hereinafter, an embodiment (first embodiment) of the method of evaluating gastric cancer of the present invention and an embodiment (second embodiment) of the gastric cancer-evaluating apparatus, the gastric cancer-evaluating method, the gastric cancer-evaluating system, the gastric cancer-evaluating program and the recording medium of the present invention are described in detail with reference to the drawings. The present invention is not limited to these embodiments.

### First Embodiment

### 1-1. Outline of the Invention

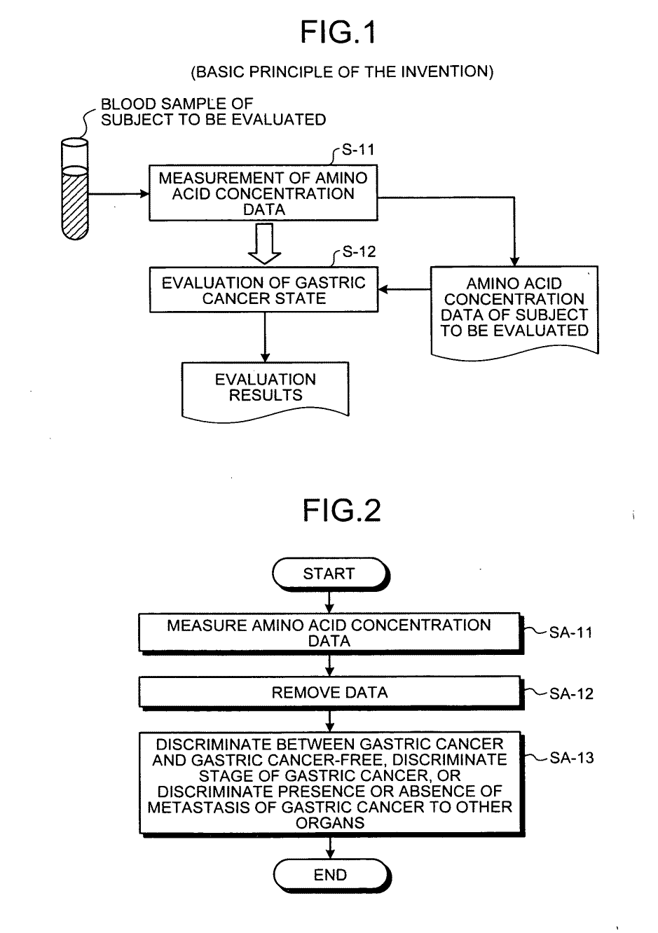

Here, an outline of the method of evaluating gastric cancer of the present invention will be described with reference to FIG. 1. FIG. 1 is a principle configurational diagram showing the basic principle of the present invention.

In the present invention, amino acid concentration data on a concentration value of an amino acid in blood collected from a subject (for example, an individual such as animal or human) to be evaluated are first measured (step S-11). Concentrations of amino acids in blood are analyzed in the following manner. A blood sample is collected in a heparin-treated tube, and then the blood plasma is separated by centrifugation of the collected blood sample. All blood plasma samples separated are frozen and stored at −70° C. before a measurement of amino acid concentrations. Before the measurement of amino acid concentrations, the blood plasma samples are deproteinized by adding sulfosalicylic acid to a concentration of 3%. An amino acid analyzer by high-performance liquid chromatography (HPLC) by using ninhydrin reaction in the post column is used for the measurement of amino acid concentrations. The unit of the amino acid concentration may be for example molar concentration, weight concentration, or these concentrations which are subjected to addition, subtraction, multiplication and division by an arbitrary constant.

In the present invention, a gastric cancer state in the subject is evaluated based on the concentration value of at least one of Asn, Cys, His, Met, Orn, Phe, Trp, Pro, Lys, Leu, Glu, Arg, Ala, Thr, and Tyr contained in the amino acid concentration data of the subject measured in the step S-11 (step S-12).

According to the present invention described above, the amino acid concentration data on the concentration value of the amino acid in blood collected from the subject is measured, and the gastric cancer state in the subject is evaluated based on the concentration value of at least one of Asn, Cys, His, Met, Orn, Phe, Trp, Pro, Lys, Leu, Glu, Arg, Ala, Thr, and Tyr contained in the measured amino acid concentration data of the subject. Thus, concentrations of amino acids which among amino acids in blood, are related to the gastric cancer state can be utilized to bring about an effect of enabling an accurate evaluation of the gastric cancer state.

Before step S-12 is executed, data such as defective and outliers may be removed from the amino acid concentration data of the subject measured in step S-11. Thereby, the gastric cancer state can be more accurately evaluated.

In step S-12, discrimination between gastric cancer and gastric cancer-free, discrimination a stage (specifically, Ia, Ib, II, IIIa, IIIb, and IV) of gastric cancer, or discrimination the presence or absence of metastasis of gastric cancer to other organs (specifically, lymph node, peritonea, liver, or the like) in the subject may be conducted based on the concentration value of at least one of Asn, Cys, His, Met, Orn, Phe, Trp, Pro, Lys, Leu, Glu, Arg, Ala, Thr, and Tyr contained in the amino acid concentration data of the subject measured in step S-11. Specifically, the concentration value of at least one of Asn, Cys, His, Met, Orn, Phe, Trp, Pro, Lys, Leu, Glu, Arg, Ala, Thr, and Tyr may be compared with a previously established threshold (cutoff value), thereby discriminating between the gastric cancer and the gastric cancer-free, discriminating the stage of gastric cancer, or discriminating the presence or absence of metastasis of gastric cancer to other organs in the subject. Thus, concentrations of amino acids which among amino acids in blood, are useful for discriminating between the 2 groups of the gastric cancer and the gastric cancer-free, discriminating the stage of gastric cancer, or discriminating between the 2 groups of the presence of metastasis of gastric cancer to other organs and the absence of the metastasis can be utilized to bring about an effect of enabling accurately these discriminations.

In step S-12, a discriminant value that is a value of a multivariate discriminant with a concentration of the amino acid as an explanatory variable may be calculated based on both (a) the concentration value of at least one of Asn, Cys, His, Met, Orn, Phe, Trp, Pro, Lys, Leu, Glu, Arg, Ala, Thr, and Tyr contained in the amino acid concentration data of the subject measured in step S-11 and (b) the previously established multivariate discriminant containing at least one of Asn, Cys, His, Met, Orn, Phe, Trp, Pro, Lys, Leu, Glu, Arg, Ala, Thr, and Tyr as the explanatory variable, and the gastric cancer state in the subject may be evaluated based on the calculated discriminant value. Thus, discriminant values obtained in multivariate discriminants which are correlated significantly with the gastric cancer state can be utilized to bring about an effect of enabling an accurate evaluation of the gastric cancer state.

In step S-12, the discriminant value that is the value of the multivariate discriminant may be calculated based on both (a) the concentration value of at least one of Asn, Cys, His, Met, Orn, Phe, Trp, Pro, Lys, Leu, Glu, Arg, Ala, Thr, and Tyr contained in the amino acid concentration data of the subject measured in step S-11 and (b) the previously established multivariate discriminant containing at least one of Asn, Cys, His, Met, Orn, Phe, Trp, Pro, Lys, Leu, Glu, Arg, Ala, Thr, and Tyr as the explanatory variable, the discrimination between the gastric cancer and the gastric cancer-free, the discrimination the stage of gastric cancer, or the discrimination the presence or absence of metastasis of gastric cancer to other organs in the subject may be conducted based on the calculated discriminant value. Specifically, the discriminant value may be compared with a previously established threshold (cutoff value), thereby discriminating between the gastric cancer and the gastric cancer-free, discriminating the stage of gastric cancer, or discriminating the presence or absence of metastasis of gastric cancer to other organs in the subject. Thus, discriminant values obtained in multivariate discriminants useful for discriminating between the 2 groups of the gastric cancer and the gastric cancer-free, discriminating the stage of gastric cancer, or discriminating between the 2 groups of the presence of metastasis of gastric cancer to other organs and the absence of the metastasis can be utilized to bring about an effect of enabling accurately these discriminations.

The multivariate discriminant may be expressed by one fractional expression or the sum of a plurality of the fractional expressions and may contain at least one of Asn, Cys, His, Met, Orn, Phe, Trp, Pro, Lys, Leu, Glu, Arg, Ala, Thr, and Tyr as the explanatory variable in any one of the numerator and denominator or both in the fractional expression constituting the multivariate discriminant. Specifically, (a) when the discrimination between the gastric cancer and the gastric cancer-free is conducted in step S-12, the multivariate discriminant may be formula 1, 2, or 3, (b) when the discrimination of the stage of gastric cancer is conducted in step S-12, the multivariate discriminant may be formula 4, and (c) when the discrimination of the presence or absence of metastasis of gastric cancer to other organs is conducted in step S-1l , the multivariate discriminant may be formula 5:

a₁×Orn/(Trp+His)+b₁×(ABA+Ile)/Leu+c₁ (formula 1)

a₂×Glu/His+b₂×Ser/Trp+c₂×Arg/Pro+d₂ (formula 2)

a₃×Trp/Gln+b₃×His/Glu+c₃ (formula 3)

a₄×Gly/(Glu+Trp+Val)+b₄×Arg/His+c₄ (formula 4)

a₅×Ile/Glu+b₅×(Gly+Asn+Arg)/His+c₅ (formula 5)

wherein a₁ and b₁ in the formula 1 are arbitrary non-zero real numbers, c₁ in the formula 1 is arbitrary real number, a₂, b₂, and c₂ in the formula 2 are arbitrary non-zero real numbers, d₂ in the formula 2 is arbitrary real number, a₃ and b₃ in the formula 3 are arbitrary non-zero real numbers, c₃ in the formula 3 is arbitrary real number, a₄ and b₄ in the formula 4 are arbitrary non-zero real numbers, c₄ in the formula 4 is arbitrary real number, a₅ and b₅ in the formula 5 are arbitrary non-zero real numbers, and c₅ in the formula 5 is arbitrary real number. Thus, discriminant values obtained in multivariate discriminants useful particularly for discriminating between the 2 groups of the gastric cancer and the gastric cancer-free, discriminating the stage of gastric cancer, or discriminating between the 2 groups of the presence of metastasis of gastric cancer to other organs and the absence of the metastasis can be utilized to bring about an effect of enabling more accurately these discriminations. The multivariate discriminants described above can be prepared by a method described in International Publication WO 2004/052191 Pamphlet that is an international application filed by the present applicant or by a method (multivariate discriminant-preparing processing described in the second embodiment described later) described in International Publication WO 2006/098192 Pamphlet that is an international application filed by the present applicant. Any multivariate discriminants obtained by these methods can be preferably used in the evaluation of the gastric cancer state, regardless of the unit of the amino acid concentration in the amino acid concentration data as input data.

In a fractional expression, the numerator of the fractional expression is expressed by the sum of the amino acids A, B, C etc. and the denominator of the fractional expression is expressed by the sum of the amino acids a, b, c etc. The fractional expression also includes the sum of the fractional expressions α, β, γ etc. (for example, α+β) having such constitution. The fractional expression also includes divided fractional expressions. The amino acids used in the numerator or denominator may have suitable coefficients respectively. The amino acids used in the numerator or denominator may appear repeatedly. Each fractional expression may have a suitable coefficient. A value of a coefficient for each explanatory variable and a value for a constant term may be any real numbers. In combinations where explanatory variables in the numerator and explanatory variables in the denominator in the fractional expression are switched with each other, the positive (or negative) sign is generally reversed in correlation with objective explanatory variables, but because their correlation is maintained, such combinations can be assumed to be equivalent to one another in discrimination, and thus the fractional expression also includes combinations where explanatory variables in the numerator and explanatory variables in the denominator in the fractional expression are switched with each other.

The multivariate discriminant may be any one of a logistic regression equation, a linear discriminant, a multiple regression equation, a discriminant prepared by a support vector machine, a discriminant prepared by a Mahalanobis' generalized distance method, a discriminant prepared by canonical discriminant analysis, and a discriminant prepared by a decision tree. Specifically, the multivariate discriminant may be the logistic regression equation with Orn, Gln, Trp, and Cit as the explanatory variables, the linear discriminant with Orn, Gln, Trp, Phe, Cit, and Try as the explanatory variables, the logistic regression equation with Glu, Phe, His, and Trp as the explanatory variables, the linear discriminant with Glu, Pro, His, and Trp as the explanatory variables, the logistic regression equation with Val, Ile, His, and Trp as the explanatory variables, or the linear discriminant with Thr, Ile, His, and Trp as the explanatory variables. Thus, discriminant values obtained in multivariate discriminants useful particularly for discriminating between the 2 groups of the gastric cancer and the gastric cancer-free, discriminating the stage of gastric cancer, or discriminating between the 2 groups of the presence of metastasis of gastric cancer to other organs and the absence of the metastasis can be utilized to bring about an effect of enabling more accurately these discriminations. The multivariate discriminants described above can be prepared by a method (multivariate discriminant-preparing processing described in the second embodiment described later) described in International Publication WO 2006/098192 Pamphlet that is an international application filed by the present applicant. Any multivariate discriminants obtained by this method can be preferably used in the evaluation of the gastric cancer state, regardless of the unit of the amino acid concentration in the amino acid concentration data as input data.

The multivariate discriminant refers to a form of equation used generally in multivariate analysis and includes, for example, multiple regression equation, multiple logistic regression equation, linear discriminant function, Mahalanobis' generalized distance, canonical discriminant function, support vector machine, and decision tree. The multivariate discriminant also includes an equation shown by the sum of different forms of multivariate discriminants. In the multiple regression equation, multiple logistic regression equation and canonical discriminant function, a coefficient and constant term are added to each explanatory variable, and the coefficient and constant term in this case are preferably real numbers, more preferably values in the range of 99% confidence interval for the coefficient and constant term obtained from data for discrimination, more preferably in the range of 95% confidence interval for the coefficient and constant term obtained from data for discrimination. The value of each coefficient and the confidence interval thereof may be those multiplied by a real number, and the value of each constant term and the confidence interval thereof may be those having an arbitrary actual constant added or subtracted or those multiplied or divided by an arbitrary actual constant.

When the gastric cancer state is evaluated (specifically, for example, the discrimination between the gastric cancer and the gastric cancer-free is conducted, the discrimination of the stage of gastric cancer is conducted, or the discrimination of the presence or absence of metastasis of gastric cancer to other organs is conducted) in the present invention, concentrations of other metabolites (biological metabolites), protein expression level, age and sex of the subject, biological indices or the like may be used in addition to the concentrations of the amino acids. When the gastric cancer state is evaluated (specifically, for example, the discrimination between the gastric cancer and the gastric cancer-free is conducted, the discrimination of the stage of gastric cancer is conducted, or the discrimination of the presence or absence of metastasis of gastric cancer to other organs is conducted) in the present invention, concentrations of other metabolites (biological metabolites), protein expression level, age and sex of the subject, biological indices or the like may be used as the explanatory variables in the multivariate discriminants in addition to the concentrations of the amino acids.

### 1-2. Method of Evaluating Gastric Cancer in Accordance With the First Embodiment

Herein, the method of evaluating gastric cancer according to the first embodiment is described with reference to FIG. 2. FIG. 2 is a flowchart showing one example of the method of evaluating gastric cancer according to the first embodiment.

The amino acid concentration data on the concentration values of amino acids is measured from blood collected from an individual such as animal or human (step SA-11). The measurement of the concentration values of the amino acids is conducted by the method described above.

Data such as defective and outliers is then removed from the amino acid concentration data of the individual measured in the step SA-11 (step SA-12).

Then, (i) the concentration value of at least one of Asn, Cys, His, Met, Orn, Phe, Trp, Pro, Lys, Leu, Glu, Arg, Ala, Thr, and Tyr contained in the amino acid concentration data of the individual from which the data such as the defective and the outliers have been removed in step SA-12 is compared with a previously established threshold (cutoff value), thereby discriminating between the gastric cancer and the gastric cancer-free, discriminating the stage of gastric cancer, or discriminating the presence or absence of metastasis of gastric cancer to other organs in the individual, or (ii) the discriminant value is calculated based on based on both (a) the concentration value of at least one of Asn, Cys, His, Met, Orn, Phe, Trp, Pro, Lys, Leu, Glu, Arg, Ala, Thr, and Tyr contained in the measured amino acid concentration data of the individual from which the data such as the defective and the outliers have been removed in step SA-12 and (b) the previously established multivariate discriminant containing at least one of Asn, Cys, His, Met, Orn, Phe, Trp, Pro, Lys, Leu, Glu, Arg, Ala, Thr, and Tyr as the explanatory variable, and the calculated discriminant value is compared with a previously established threshold (cutoff value), thereby discriminating between the gastric cancer and the gastric cancer-free, discriminating the stage of gastric cancer, or discriminating the presence or absence of metastasis of gastric cancer to other organs in the individual (step SA-13).

### 1-3. Summary of the First Embodiment and Other Embodiments

In the method of evaluating gastric cancer according to the first embodiment as described above in detail, (1) the amino acid concentration data is measured from blood collected from the individual, (2) the data such as the defective and the outliers is removed from the measured amino acid concentration data of the individual, and (3) (i) the concentration value of at least one of Asn, Cys, His, Met, Orn, Phe, Trp, Pro, Lys, Leu, Glu, Arg, Ala, Thr, and Tyr contained in the amino acid concentration data of the individual from which the data such as the defective and the outliers have been removed is compared with the previously established threshold (cutoff value), thereby discriminating between the gastric cancer and the gastric cancer-free, discriminating the stage of gastric cancer, or discriminating the presence or absence of metastasis of gastric cancer to other organs in the individual, or (ii) the discriminant value is calculated based on based on both (a) the concentration value of at least one of Asn, Cys, His, Met, Orn, Phe, Trp, Pro, Lys, Leu, Glu, Arg, Ala, Thr, and Tyr contained in the measured amino acid concentration data of the individual from which the data such as the defective and the outliers have been removed and (b) the previously established multivariate discriminant containing at least one of Asn, Cys, His, Met, Orn, Phe, Trp, Pro, Lys, Leu, Glu, Arg, Ala, Thr, and Tyr as the explanatory variable, and the calculated discriminant value is compared with the previously established threshold (cutoff value), thereby discriminating between the gastric cancer and the gastric cancer-free, discriminating the stage of gastric cancer, or discriminating the presence or absence of metastasis of gastric cancer to other organs in the individual. Thus, concentrations of amino acids which among amino acids in blood, are useful for discriminating between the 2 groups of the gastric cancer and the gastric cancer-free, discriminating the stage of gastric cancer, or discriminating between the 2 groups of the presence of metastasis of gastric cancer to other organs and the absence of the metastasis or discriminant values obtained in multivariate discriminants useful for these discriminations can be utilized to bring about an effect of enabling accurately these discriminations.

In step SA-13, the multivariate discriminant may be expressed by one fractional expression or the sum of a plurality of the fractional expressions and may contain at least one of Asn, Cys, His, Met, Orn, Phe, Trp, Pro, Lys, Leu, Glu, Arg, Ala, Thr, and Tyr as the explanatory variable in any one of the numerator and denominator or both in the fractional expression constituting the multivariate discriminant. Specifically, (a) when the discrimination between the gastric cancer and the gastric cancer-free is conducted in step SA-13, the multivariate discriminant may be formula 1, 2, or 3, (b) when the discrimination of the stage of gastric cancer is conducted in step SA-13, the multivariate discriminant may be formula 4, and (c) when the discrimination of the presence or absence of metastasis of gastric cancer to other organs is conducted in step SA-13, the multivariate discriminant may be formula 5:

a₁×Orn/(Trp+His)+b₁×(ABA+Ile)/Leu+c₁ (formula 1)

a₂×Glu/His+b₂×Ser/Trp+c₂×Arg/Pro+d₂ (formula 2)

a₃×Trp/Gln+b₃×His/Glu+c₃ (formula 3)

a₄×Gly/(Glu+Trp+Val)+b₄×Arg/His+c₄ (formula 4)

a₅×Ile/Glu+b₅×(Gly+Asn+Arg)/His+c₅ (formula 5)

wherein a₁ and b₁ in the formula 1 are arbitrary non-zero real numbers, c₁ in the formula 1 is arbitrary real number, a₂, b₂, and c₂ in the formula 2 are arbitrary non-zero real numbers, d₂ in the formula 2 is arbitrary real number, a₃ and b₃ in the formula 3 are arbitrary non-zero real numbers, c₃ in the formula 3 is arbitrary real number, a₄ and b₄ in the formula 4 are arbitrary non-zero real numbers, c₄ in the formula 4 is arbitrary real number, a₅ and b₅ in the formula 5 are arbitrary non-zero real numbers, and c₅ in the formula 5 is arbitrary real number. Thus, discriminant values obtained in multivariate discriminants useful particularly for discriminating between the 2 groups of the gastric cancer and the gastric cancer-free, discriminating the stage of gastric cancer, or discriminating between the 2 groups of the presence of metastasis of gastric cancer to other organs and the absence of the metastasis can be utilized to bring about an effect of enabling more accurately these discriminations. The multivariate discriminants described above can be prepared by a method described in International Publication WO 2004/052191 Pamphlet that is an international application filed by the present applicant or by a method (multivariate discriminant-preparing processing described in the second embodiment described later) described in International Publication WO 2006/098192 Pamphlet that is an international application filed by the present applicant. Any multivariate discriminants obtained by these methods can be preferably used in the evaluation of the gastric cancer state, regardless of the unit of the amino acid concentration in the amino acid concentration data as input data.

In step SA-13, the multivariate discriminant may be any one of a logistic regression equation, a linear discriminant, a multiple regression equation, a discriminant prepared by a support vector machine, a discriminant prepared by a Mahalanobis' generalized distance method, a discriminant prepared by canonical discriminant analysis, and a discriminant prepared by a decision tree. Specifically, the multivariate discriminant may be the logistic regression equation with Orn, Gln, Trp, and Cit as the explanatory variables, the linear discriminant with Orn, Gln, Trp, Phe, Cit, and Try as the explanatory variables, the logistic regression equation with Glu, Phe, His, and Trp as the explanatory variables, the linear discriminant with Glu, Pro, His, and Trp as the explanatory variables, the logistic regression equation with Val, Ile, His, and Trp as the explanatory variables, or the linear discriminant with Thr, Ile, His, and Trp as the explanatory variables. Thus, discriminant values obtained in multivariate discriminants useful particularly for discriminating between the 2 groups of the gastric cancer and the gastric cancer-free, discriminating the stage of gastric cancer, or discriminating between the 2 groups of the presence of metastasis of gastric cancer to other organs and the absence of the metastasis can be utilized to bring about an effect of enabling more accurately these discriminations. The multivariate discriminants described above can be prepared by a method (multivariate discriminant-preparing processing described in the second embodiment described later) described in International Publication WO 2006/098192 Pamphlet that is an international application filed by the present applicant. Any multivariate discriminants obtained by this method can be preferably used in the evaluation of the gastric cancer state, regardless of the unit of the amino acid concentration in the amino acid concentration data as input data.

### Second Embodiment

### 2-1. Outline of the Invention

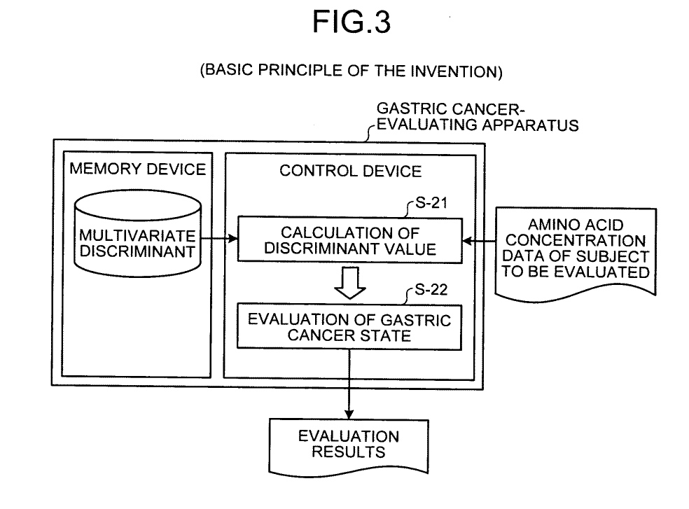

Herein, an outline of the gastric cancer-evaluating apparatus, the gastric cancer-evaluating method, the gastric cancer-evaluating system, the gastric cancer-evaluating program and the recording medium of the present invention are described in detail with reference to FIG. 3. FIG. 3 is a principle configurational diagram showing the basic principle of the present invention.

In the present invention, a discriminant value that is a value of a multivariate discriminant with a concentration of an amino acid as an explanatory variable is calculated in a control device based on both (a) a concentration value of at least one of Asn, Cys, His, Met, Orn, Phe, Trp, Pro, Lys, Leu, Glu, Arg, Ala, Thr, and Tyr contained in previously obtained amino acid concentration data of a subject (for example, an individual such as animal or human) to be evaluated on the concentration value of the amino acid and (b) the multivariate discriminant containing at least one of Asn, Cys, His, Met, Orn, Phe, Trp, Pro, Lys, Leu, Glu, Arg, Ala, Thr, and Tyr as the explanatory variable, stored in the memory device (step S-21).

In the present invention, a gastric cancer state in the subject is evaluated in the control device based on the discriminant value calculated in step S-21 (step S-22).

According to the present invention described above, the discriminant value that is the value of the multivariate discriminant with the concentration of the amino acid as the explanatory variable is calculated based on both (a) the concentration value of at least one of Asn, Cys, His, Met, Orn, Phe, Trp, Pro, Lys, Leu, Glu, Arg, Ala, Thr, and Tyr contained in the previously obtained amino acid concentration data of the subject on the concentration value of the amino acid and (b) the multivariate discriminant containing at least one of Asn, Cys, His, Met, Orn, Phe, Trp, Pro, Lys, Leu, Glu, Arg, Ala, Thr, and Tyr as the explanatory variable stored in the memory device, and the gastric cancer state in the subject is evaluated based on the calculated discriminant value. Thus, discriminant values obtained in multivariate discriminants which are correlated significantly with the gastric cancer state can be utilized to bring about an effect of enabling an accurate evaluation of the gastric cancer state.

In step S-22, discrimination between gastric cancer and gastric cancer-free, discrimination a stage of gastric cancer, or discrimination the presence or absence of metastasis of gastric cancer to other organs in the subject may be conducted based on the discriminant value calculated in step S-21. Specifically, the discriminant value may be compared with a previously established threshold (cutoff value), thereby discriminating between the gastric cancer and the gastric cancer-free, discriminating the stage of gastric cancer, or discriminating the presence or absence of metastasis of gastric cancer to other organs in the subject. Thus, discriminant values obtained in multivariate discriminants useful for discriminating between the 2 groups of the gastric cancer and the gastric cancer-free, discriminating the stage of gastric cancer, or discriminating between the 2 groups of the presence of metastasis of gastric cancer to other organs and the absence of the metastasis can be utilized to bring about an effect of enabling accurately these discriminations.

The multivariate discriminant may be expressed by one fractional expression or the sum of a plurality of the fractional expressions and may contain at least one of Asn, Cys, His, Met, Orn, Phe, Trp, Pro, Lys, Leu, Glu, Arg, Ala, Thr, and Tyr as the explanatory variable in any one of the numerator and denominator or both in the fractional expression constituting the multivariate discriminant. Specifically, (a) when the discrimination between the gastric cancer and the gastric cancer-free is conducted in step S-22, the multivariate discriminant may be formula 1, 2, or 3, (b) when the discrimination of the stage of gastric cancer is conducted in step S-22, the multivariate discriminant may be formula 4, and (c) when the discrimination of the presence or absence of metastasis of gastric cancer to other organs is conducted in step S-2l , the multivariate discriminant may be formula 5:

a₁×Orn/(Trp+His)+b₁×(ABA+Ile)/Leu+c₁ (formula 1)

a₂×Glu/His+b₂×Ser/Trp+c₂×Arg/Pro+d₂ (formula 2)

a₃×Trp/Gln+b₃×His/Glu+c₃ (formula 3)

a₄×Gly/(Glu+Trp+Val)+b₄×Arg/His+c₄ (formula 4)

a₅×Ile/Glu+b₅×(Gly+Asn+Arg)/His+c₅ (formula 5)

wherein a₁ and b₁ in the formula 1 are arbitrary non-zero real numbers, c₁ in the formula 1 is arbitrary real number, a₂, b₂, and c₂ in the formula 2 are arbitrary non-zero real numbers, d₂ in the formula 2 is arbitrary real number, a₃ and b₃ in the formula 3 are arbitrary non-zero real numbers, c₃ in the formula 3 is arbitrary real number, a₄ and b₄ in the formula 4 are arbitrary non-zero real numbers, c₄ in the formula 4 is arbitrary real number, a₅ and b₅ in the formula 5 are arbitrary non-zero real numbers, and c₅ in the formula 5 is arbitrary real number. Thus, discriminant values obtained in multivariate discriminants useful particularly for discriminating between the 2 groups of the gastric cancer and the gastric cancer-free, discriminating the stage of gastric cancer, or discriminating between the 2 groups of the presence of metastasis of gastric cancer to other organs and the absence of the metastasis can be utilized to bring about an effect of enabling more accurately these discriminations. The multivariate discriminants described above can be prepared by a method described in International Publication WO 2004/052191 Pamphlet that is an international application filed by the present applicant or by a method (multivariate discriminant-preparing processing described later) described in International Publication WO 2006/098192 Pamphlet that is an international application filed by the present applicant. Any multivariate discriminants obtained by these methods can be preferably used in the evaluation of the gastric cancer state, regardless of the unit of the amino acid concentration in the amino acid concentration data as input data.

In a fractional expression, the numerator of the fractional expression is expressed by the sum of the amino acids A, B, C etc. and the denominator of the fractional expression is expressed by the sum of the amino acids a, b, c etc. The fractional expression also includes the sum of the fractional expressions α, β, γ etc. (for example, α+β) having such constitution. The fractional expression also includes divided fractional expressions. The amino acids used in the numerator or denominator may have suitable coefficients respectively. The amino acids used in the numerator or denominator may appear repeatedly. Each fractional expression may have a suitable coefficient. A value of a coefficient for each explanatory variable and a value for a constant term may be any real numbers. In combinations where explanatory variables in the numerator and explanatory variables in the denominator in the fractional expression are switched with each other, the positive (or negative) sign is generally reversed in correlation with objective explanatory variables, but because their correlation is maintained, such combinations can be assumed to be equivalent to one another in discrimination, and thus the fractional expression also includes combinations where explanatory variables in the numerator and explanatory variables in the denominator in the fractional expression are switched with each other.

The multivariate discriminant may be any one of a logistic regression equation, a linear discriminant, a multiple regression equation, a discriminant prepared by a support vector machine, a discriminant prepared by a Mahalanobis' generalized distance method, a discriminant prepared by canonical discriminant analysis, and a discriminant prepared by a decision tree. Specifically, the multivariate discriminant may be the logistic regression equation with Orn, Gln, Trp, and Cit as the explanatory variables, the linear discriminant with Orn, Gln, Trp, Phe, Cit, and Try as the explanatory variables, the logistic regression equation with Glu, Phe, His, and Trp as the explanatory variables, the linear discriminant with Glu, Pro, His, and Trp as the explanatory variables, the logistic regression equation with Val, Ile, His, and Trp as the explanatory variables, or the linear discriminant with Thr, Ile, His, and Trp as the explanatory variables. Thus, discriminant values obtained in multivariate discriminants useful particularly for discriminating between the 2 groups of the gastric cancer and the gastric cancer-free, discriminating the stage of gastric cancer, or discriminating between the 2 groups of the presence of metastasis of gastric cancer to other organs and the absence of the metastasis can be utilized to bring about an effect of enabling more accurately these discriminations. The multivariate discriminants described above can be prepared by a method (multivariate discriminant-preparing processing described later) described in International Publication WO 2006/098192 Pamphlet that is an international application filed by the present applicant. Any multivariate discriminants obtained by this method can be preferably used in the evaluation of the gastric cancer state, regardless of the unit of the amino acid concentration in the amino acid concentration data as input data.

The multivariate discriminant refers to a form of equation used generally in multivariate analysis and includes, for example, multiple regression equation, multiple logistic regression equation, linear discriminant function, Mahalanobis' generalized distance, canonical discriminant function, support vector machine, and decision tree. The multivariate discriminant also includes an equation shown by the sum of different forms of multivariate discriminants. In the multiple regression equation, multiple logistic regression equation and canonical discriminant function, a coefficient and constant term are added to each explanatory variable, and the coefficient and constant term in this case are preferably real numbers, more preferably values in the range of 99% confidence interval for the coefficient and constant term obtained from data for discrimination, more preferably in the range of 95% confidence interval for the coefficient and constant term obtained from data for discrimination. The value of each coefficient and the confidence interval thereof may be those multiplied by a real number, and the value of each constant term and the confidence interval thereof may be those having an arbitrary actual constant added or subtracted or those multiplied or divided by an arbitrary actual constant.

When the gastric cancer state is evaluated (specifically, for example, the discrimination between the gastric cancer and the gastric cancer-free is conducted, the discrimination of the stage of gastric cancer is conducted, or the discrimination of the presence or absence of metastasis of gastric cancer to other organs is conducted) in the present invention, concentrations of other metabolites (biological metabolites), protein expression level, age and sex of the subject, biological indices or the like may be used in addition to the concentrations of the amino acids. When the gastric cancer state is evaluated (specifically, for example, the discrimination between the gastric cancer and the gastric cancer-free is conducted, the discrimination of the stage of gastric cancer is conducted, or the discrimination of the presence or absence of metastasis of gastric cancer to other organs is conducted) in the present invention, concentrations of other metabolites (biological metabolites), protein expression level, age and sex of the subject, biological indices or the like may be used as the explanatory variables in the multivariate discriminants in addition to the concentrations of the amino acids.

Here, the summary of the multivariate discriminant-preparing processing (steps 1 to 4) is described in detail.

First, a candidate multivariate discriminant (e.g., y=a₁x₁+a₂x₂+ . . . +aₙxₙ, y: gastric cancer state index data, xᵢ: amino acid concentration data, constant, i=1, 2, . . . n) that is a candidate of the multivariate discriminant is prepared in the control device based on a predetermined discriminant-preparing method from gastric cancer state information stored in a memory device containing the amino acid concentration data and gastric cancer state index data on an index for indicating the gastric cancer state (step 1). Data containing defective and outliers may be removed in advance from the gastric cancer state information.

In step 1, a plurality of the candidate multivariate discriminants may be prepared from the gastric cancer state information by using a plurality of the different discriminant-preparing methods (including those for multivariate analysis such as principal component analysis, discriminant analysis, support vector machine, multiple regression analysis, logistic regression analysis, k-means method, cluster analysis, and decision tree). Specifically, a plurality of the candidate multivariate discriminant groups may be prepared simultaneously and concurrently by using a plurality of different algorithms with the gastric cancer state information which is multivariate data composed of the amino acid concentration data and the gastric cancer state index data obtained by analyzing blood samples from a large number of healthy subjects and gastric cancer patients. For example, the two different candidate multivariate discriminants may be formed by performing discriminant analysis and logistic regression analysis simultaneously with the different algorithms. Alternatively, the candidate multivariate discriminant may be formed by converting the gastric cancer state information with the candidate multivariate discriminant prepared by performing principal component analysis and then performing discriminant analysis of the converted gastric cancer state information. In this way, it is possible to finally prepare the multivariate discriminant suitable for diagnostic condition.

The candidate multivariate discriminant prepared by principal component analysis is a linear expression consisting of amino acid explanatory variables maximizing the variance of all amino acid concentration data. The candidate multivariate discriminant prepared by discriminant analysis is a high-powered expression (including exponential and logarithmic expressions) consisting of amino acid explanatory variables minimizing the ratio of the sum of the variances in respective groups to the variance of all amino acid concentration data. The candidate multivariate discriminant prepared by using support vector machine is a high-powered expression (including kernel function) consisting of amino acid explanatory variables maximizing the boundary between groups. The candidate multivariate discriminant prepared by multiple regression analysis is a high-powered expression consisting of amino acid explanatory variables minimizing the sum of the distances from all amino acid concentration data. The candidate multivariate discriminant prepared by logistic regression analysis is a fraction expression having, as a component, the natural logarithm having a linear expression consisting of amino acid explanatory variables maximizing the likelihood as the exponent. The k-means method is a method of searching k pieces of neighboring amino acid concentration data in various groups, designating the group containing the greatest number of the neighboring points as its data-belonging group, and selecting the amino acid explanatory variable that makes the group to which input amino acid concentration data belong agree well with the designated group. The cluster analysis is a method of clustering (grouping) the points closest in entire amino acid concentration data. The decision tree is a method of ordering amino acid explanatory variables and predicting the group of amino acid concentration data from the pattern possibly held by the higher-ordered amino acid explanatory variable.

Returning to the description of the multivariate discriminant-preparing processing, the candidate multivariate discriminant prepared in step 1 is verified (mutually verified) in the control device based on a particular verifying method (step 2). The verification of the candidate multivariate discriminant is performed on each other to each candidate multivariate discriminant prepared in step 1.

In step 2, at least one of discrimination rate, sensitivity, specificity, information criterion, and the like of the candidate multivariate discriminant may be verified by at least one of the bootstrap method, holdout method, leave-one-out method, and the like. In this way, it is possible to prepare the candidate multivariate discriminant higher in predictability or reliability, by taking the gastric cancer state information and the diagnostic condition into consideration.

The discrimination rate is the rate of the gastric cancer states judged correct according to the present invention in all input data. The sensitivity is the rate of the gastric cancer states judged correct according to the present invention in the gastric cancer states declared in the input data. The specificity is the rate of the gastric cancer states judged correct according to the present invention in the gastric cancer states declared healthy in the input data. The information criterion is the sum of the number of the amino acid explanatory variables in the candidate multivariate discriminant prepared in the step 1 and the difference in number between the gastric cancer states evaluated according to the present invention and those declared in input data. The predictability is the average of the discrimination rate, sensitivity, or specificity obtained by repeating verification of the candidate multivariate discriminant. Alternatively, the reliability is the variance of the discrimination rate, sensitivity, or specificity obtained by repeating verification of the candidate multivariate discriminant.

Returning to the description of the multivariate discriminant-preparing processing, a combination of the amino acid concentration data contained in the gastric cancer state information used in preparing the candidate multivariate discriminant is selected by selecting the explanatory variable of the candidate multivariate discriminant in the control device based on a predetermined explanatory variable-selecting method from the verification result obtained in the step 2 (step 3). The selection of the amino acid explanatory variable is performed on each candidate multivariate discriminant prepared in the step 1.

In this way, it is possible to select the amino acid explanatory variable of the candidate multivariate discriminant properly. The step 1 is executed once again by using the gastric cancer state information including the amino acid concentration data selected in the step 3.

In the step 3, the amino acid explanatory variable of the candidate multivariate discriminant may be selected based on at least one of the stepwise method, best path method, local search method, and genetic algorithm from the verification result obtained in the step 2.

The best path method is a method of selecting an amino acid explanatory variable by optimizing an evaluation index of the candidate multivariate discriminant while eliminating the amino acid explanatory variables contained in the candidate multivariate discriminant one by one.

Returning to the description of the multivariate discriminant-preparing processing, the steps 1, 2 and 3 are repeatedly performed in the control device, and based on verification results thus accumulated, the candidate multivariate discriminant used as the multivariate discriminant is selected from a plurality of the candidate multivariate discriminants, thereby preparing the multivariate discriminant (step 4). In the selection of the candidate multivariate discriminants, there are cases where the optimum multivariate discriminant is selected from the candidate multivariate discriminants prepared in the same discriminant-preparing method or the optimum multivariate discriminant is selected from all candidate multivariate discriminants.

As described above, in the multivariate discriminant-preparing processing, the processing for the preparation of the candidate multivariate discriminants, the verification of the candidate multivariate discriminants, and the selection of the explanatory variables in the candidate multivariate discriminants are performed based on the gastric cancer state information in a series of operations in a systematized manner, whereby the optimum multivariate discriminant for the evaluation of gastric cancer state can be prepared.

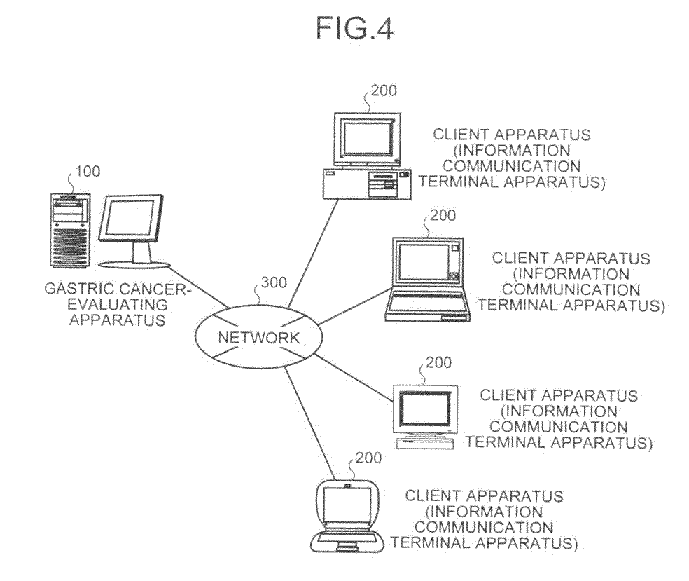

### 2-2. System Configuration Hereinafter, the configuration of the gastric cancer-evaluating system according to the second embodiment (hereinafter referred to sometimes as the present system) will be described with reference to FIGS. 4 to 20. This system is merely one example, and the present invention is not limited thereto.

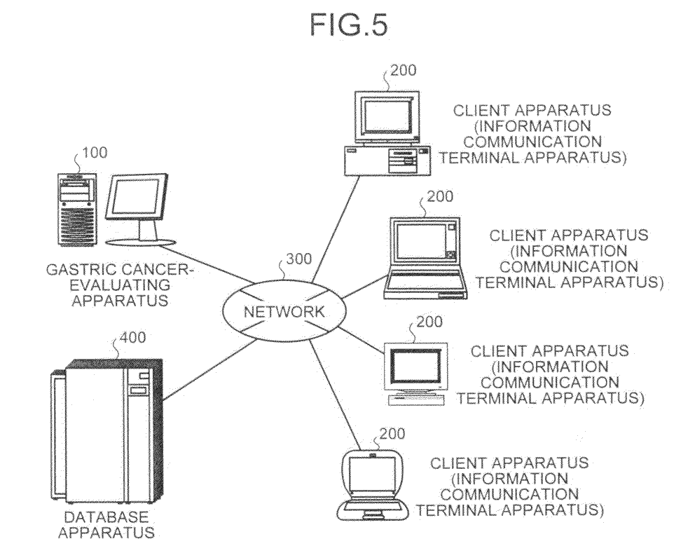

First, an entire configuration of the present system will be described with reference to FIGS. 4 and 5. FIG. 4 is a diagram showing an example of the entire configuration of the present system. FIG. 5 is a diagram showing another example of the entire configuration of the present system. As shown in FIG. 4, the present system is constituted in which the gastric cancer-evaluating apparatus 100 that evaluates the gastric cancer state in the subject, and the client apparatus 200 (corresponding to the information communication terminal apparatus of the present invention) that provides the amino acid concentration data of the subject on the concentration values of the amino acids, are communicatively connected to each other via a network 300.

In the present system as shown in FIG. 5, in addition to the gastric cancer-evaluating apparatus 100 and the client apparatus 200, the database apparatus 400 storing, for example, the gastric cancer state information used in preparing the multivariate discriminant and the multivariate discriminant used in evaluating the gastric cancer state in the gastric cancer-evaluating apparatus 100, may be communicatively connected via the network 300. In this configuration, the information on the gastric cancer state etc. are provided via the network 300 from the gastric cancer-evaluating apparatus 100 to the client apparatuses 200 and the database apparatus 400, or from the client apparatuses 200 and the database apparatus 400 to the gastric cancer-evaluating apparatus 100. The “information on the gastric cancer state” is information on measured values of particular items of the gastric cancer state of organisms including human. The information on the gastric cancer state is generated in the gastric cancer-evaluating apparatus 100, the client apparatus 200, and other apparatuses (e.g., various measuring apparatuses) and stored mainly in the database apparatus 400.

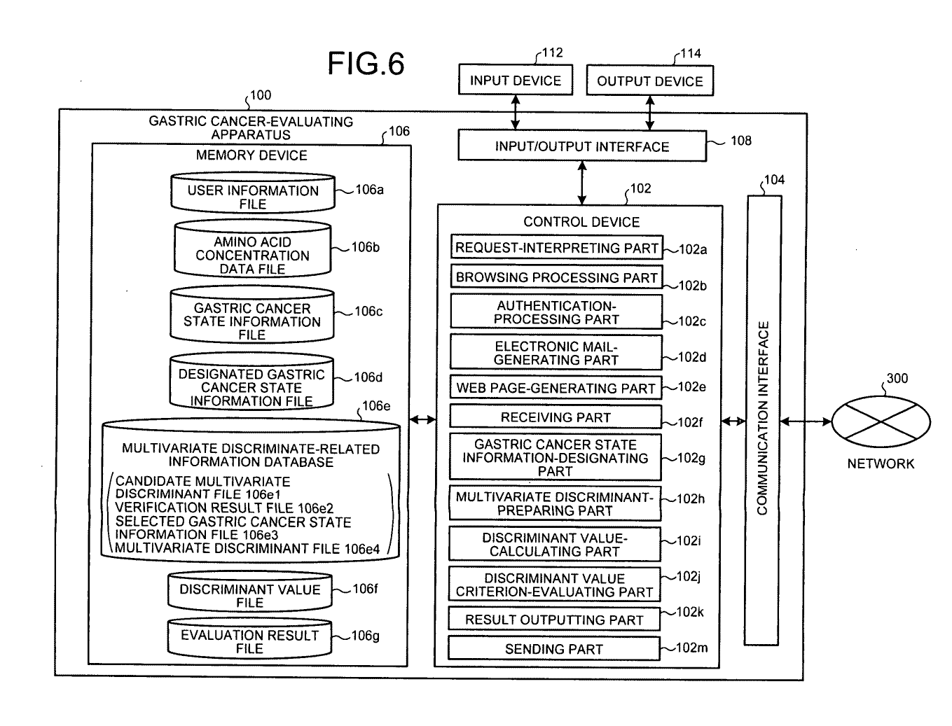

Now, the configuration of the gastric cancer-evaluating apparatus 100 in the present system will be described with reference to FIGS. 6 to 18. FIG. 6 is a block diagram showing an example of the configuration of the gastric cancer-evaluating apparatus 100 in the present system, showing conceptually only the region relevant to the present invention.

The gastric cancer-evaluating apparatus 100 includes (a) a control device 102, such as CPU (Central Processing Unit), that integrally controls the gastric cancer-evaluating apparatus 100, (b) a communication interface 104 that connects the gastric cancer-evaluating apparatus 100 to the network 300 communicatively via communication apparatuses such as a router and wired or wireless communication lines such as a private line, (c) a memory device 106 that stores various databases, tables, files and others, and (d) an input/output interface 108 connected to an input device 112 and an output device 114, and these parts are connected to each other communicatively via any communication channel. The gastric cancer-evaluating apparatus 100 may be present together with various analyzers (e.g., amino acid analyzer) in a same housing. A typical configuration of disintegration/integration of the gastric cancer-evaluating apparatus 100 is not limited to that shown in the figure, and all or a part of it may be disintegrated or integrated functionally or physically in any unit, for example, according to various loads applied. For example, a part of the processing may be performed via CGI (Common Gateway Interface).

The memory device 106 is a storage means, and examples thereof include memory apparatuses such as RAM (Random Access Memory) and ROM (Read Only Memory), a fixed disk drives such as a hard disk, a flexible disk, an optical disk, and the like. The memory device 106 stores computer programs giving instructions to the CPU for various processings, together with OS (Operating System). As shown in the figure, the memory device 106 stores the user information file 106a, the amino acid concentration data file 106b, the gastric cancer state information file 106c, the designated gastric cancer state information file 106d, a multivariate discriminant-related information database 106e, the discriminant value file 106f and the evaluation result file 106g.

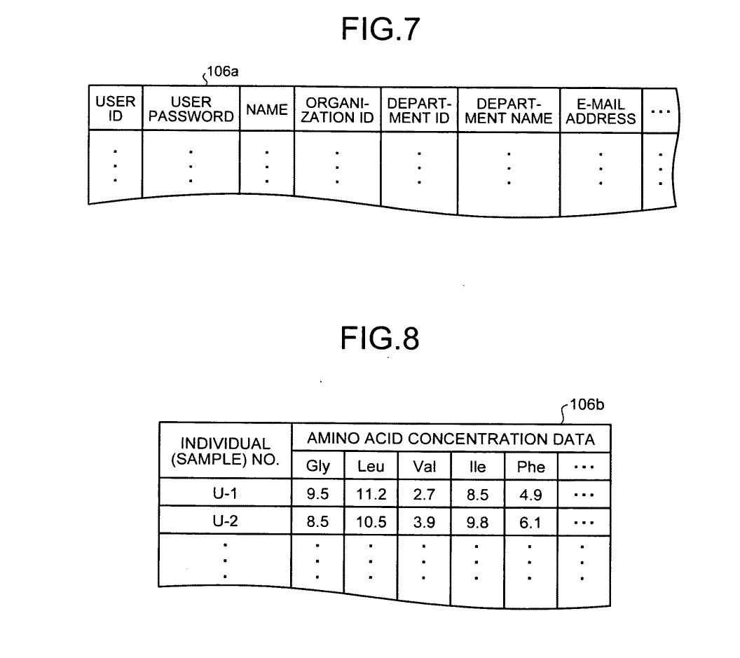

The user information file 106a stores user information on users. FIG. 7 is a chart showing an example of information stored in the user information file 106a. As shown in FIG. 7, the information stored in the user information file 106a includes user ID (identification) for identifying a user uniquely, user password for authentication of the user, user name, organization ID for uniquely identifying an organization of the user, department ID for uniquely identifying a department of the user organization, department name, and electronic mail address of the user that are correlated to one another.

Returning to FIG. 6, the amino acid concentration data file 106b stores the amino acid concentration data on the concentration values of the amino acids. FIG. 8 is a chart showing an example of the information stored in the amino acid concentration data file 106b. As shown in FIG. 8, the information stored in the amino acid concentration data file 106b includes individual number for uniquely identifying an individual (sample) as a subject to be evaluated and amino acid concentration data that are correlated to one another. In FIG. 8, the amino acid concentration data are assumed to be numerical values, i.e., on a continuous scale, but the amino acid concentration data may be expressed on a nominal scale or an ordinal scale. In the case of the nominal or ordinal scale, any number may be allocated to each state for analysis. The amino acid concentration data may be combined with other biological information (e.g., sex difference, age, smoking, digitalized electrocardiogram waveform, enzyme concentration, gene expression level, pepsinogen leve, the presence or absence of Helicobacter pylori infection, and the concentrations of metabolites other than amino acids).

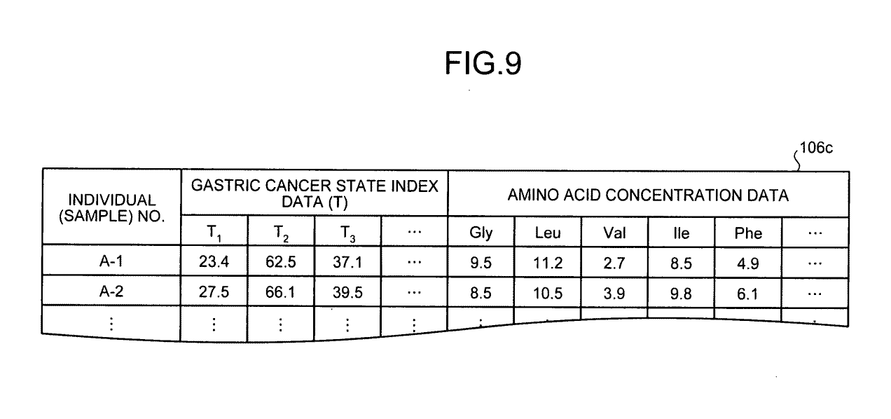

Returning to FIG. 6, the gastric cancer state information file 106c stores the gastric cancer state information used in preparing the multivariate discriminant. FIG. 9 is a chart showing an example of information stored in the gastric cancer state information file 106c. As shown in FIG. 9, the information stored in the gastric cancer state information file 106c includes individual (sample) number, gastric cancer state index data (T) corresponding to a gastric cancer state index (index T₁, index T₂, index T₃ . . . ), and amino acid concentration data that are correlated to one another. In FIG. 9, the gastric cancer state index data and the amino acid concentration data are assumed to be numerical values, i.e., on a continuous scale, but the gastric cancer state index data and the amino acid concentration data may be expressed on a nominal scale or an ordinal scale. In the case of a nominal or ordinal scale, any number may be allocated to each state for analysis. The gastric cancer state index data is a single known condition index serving as a marker of gastric cancer state, and numerical data may be used.

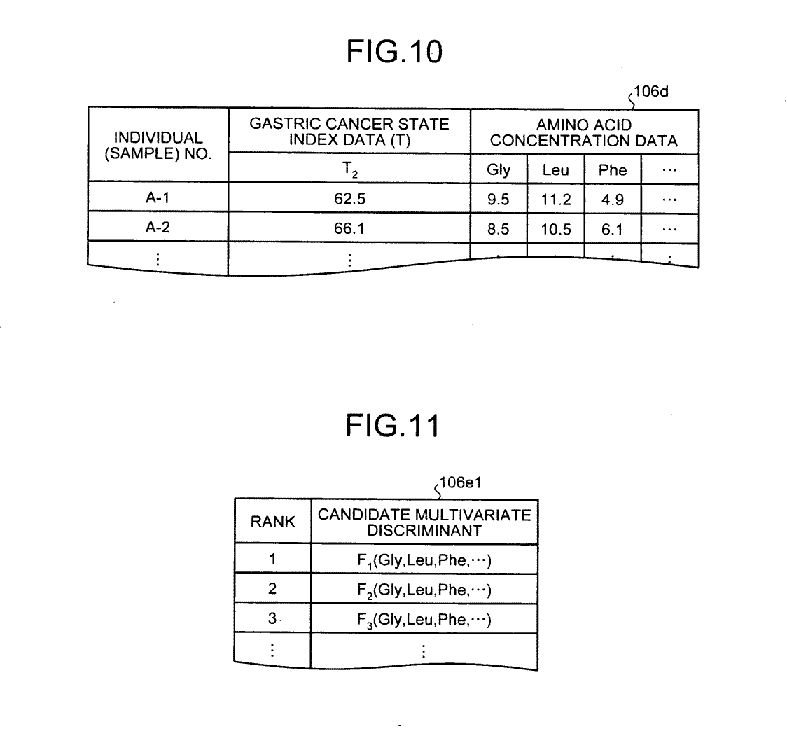

Returning to FIG. 6, the designated gastric cancer state information file 106d stores the gastric cancer state information designated in a gastric cancer state information-designating part 102g described below. FIG. 10 is a chart showing an example of information stored in the designated gastric cancer state information file 106d. As shown in FIG. 10, the information stored in the designated gastric cancer state information file 106d includes individual number, designated gastric cancer state index data, and designated amino acid concentration data that are correlated to one another.

Returning to FIG. 6, the multivariate discriminant-related information database 106e is composed of (i) the candidate multivariate discriminant file 106e1 storing the candidate multivariate discriminant prepared in a candidate multivariate discriminant-preparing part 102h1 described below, (ii) the verification result file 106e2 storing the verification results obtained in a candidate multivariate discriminant-verifying part 102h2 described below, (iii) the selected gastric cancer state information file 106e3 storing the gastric cancer state information containing the combination of the amino acid concentration data selected in an explanatory variable-selecting part 102h3 described below and (iv) the multivariate discriminant file 106e4 storing the multivariate discriminants prepared in the multivariate discriminant-preparing part 102h described below.

The candidate multivariate discriminant file 106e1 stores the candidate multivariate discriminants prepared in the candidate multivariate discriminant-preparing part 102h1 described below. FIG. 11 is a chart showing an example of information stored in the candidate multivariate discriminant file 106e1. As shown in FIG. 11, the information stored in the candidate multivariate discriminant file 106e1 includes rank, and candidate multivariate discriminant (e.g., F₁ (Gly, Leu, Phe, . . . ), F₂ (Gly, Leu, Phe, . . . ), or F₃ (Gly, Leu, Phe, . . . ) in FIG. 11) that are correlated to each other.

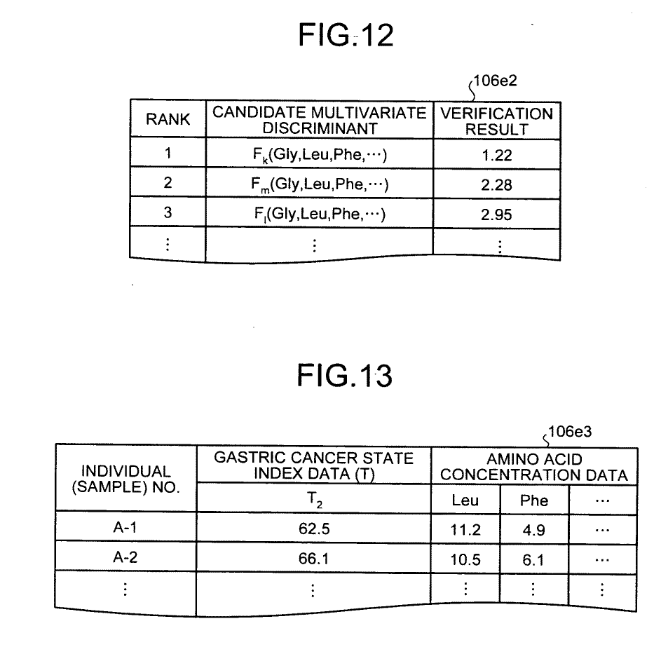

Returning to FIG. 6, the verification result file 106e2 stores the verification results obtained in the candidate multivariate discriminant-verifying part 102h2 described below. FIG. 12 is a chart showing an example of information stored in the verification result file 106e2.

As shown in FIG. 12, the information stored in the verification result file 106e2 includes rank, candidate multivariate discriminant (e.g., Fₖ (Gly, Leu, Phe, . . . ), Fₘ (Gly, Leu, Phe, . . . ), F₁ (Gly, Leu, Phe, . . . ) in FIG. 12), and verification result of each candidate multivariate discriminant (e.g., evaluation value of each candidate multivariate discriminant) that are correlated to one another.

Returning to FIG. 6, the selected gastric cancer state information file 106e3 stores the gastric cancer state information including the combination of the amino acid concentration data corresponding to the explanatory variable selected in the explanatory variable-selecting part 102h3 described below. FIG. 13 is a chart showing an example of information stored in the selected gastric cancer state information file 106e3. As shown in FIG. 13, the information stored in the selected gastric cancer state information file 106e3 includes individual number, gastric cancer state index data designated in the gastric cancer state information-designating part 102g described below, and amino acid concentration data selected in the explanatory variable-selecting part 102h3 described below that are correlated to one another.

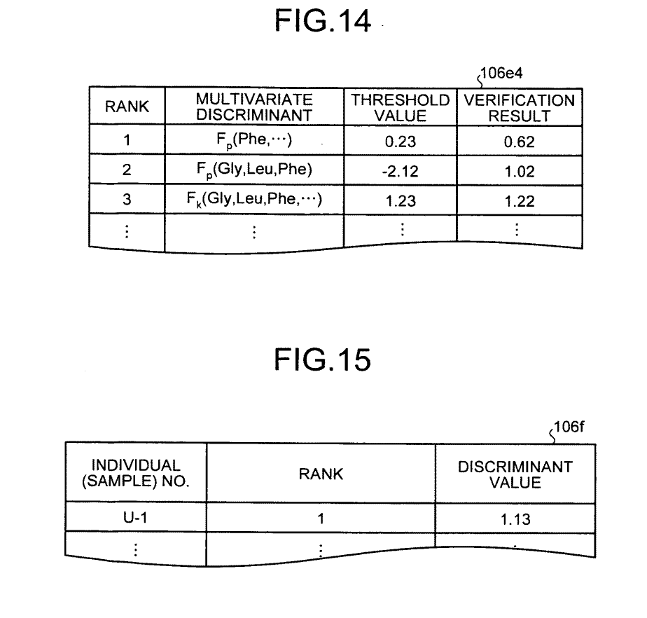

Returning to FIG. 6, the multivariate discriminant file 106e4 stores the multivariate discriminant prepared in the multivariate discriminant-preparing part 102h described below. FIG. 14 is a chart showing an example of information stored in the multivariate discriminant file 106e4. As shown in FIG. 14, the information stored in the multivariate discriminant file 106e4 includes rank, multivariate discriminant (e.g., Fₚ (Phe, . . . ), Fₚ (Gly, Leu, Phe), Fₖ (Gly, Leu, Phe, . . . ) in FIG. 14), threshold corresponding to each discriminant-preparing method, and verification result of each multivariate discriminant (e.g., evaluation value of each multivariate discriminant) that are correlated to one another.

Returning to FIG. 6, the discriminant value file 106f stores the discriminant value calculated in a discriminant value-calculating part 102i described below. FIG. 15 is a chart showing an example of information stored in the discriminant value file 106f. As shown in FIG. 15, the information stored in the discriminant value file 106f includes individual number for uniquely identifying the individual (sample) as the subject, rank (number for uniquely identifying the multivariate discriminant), and discriminant value that are correlated to one another.

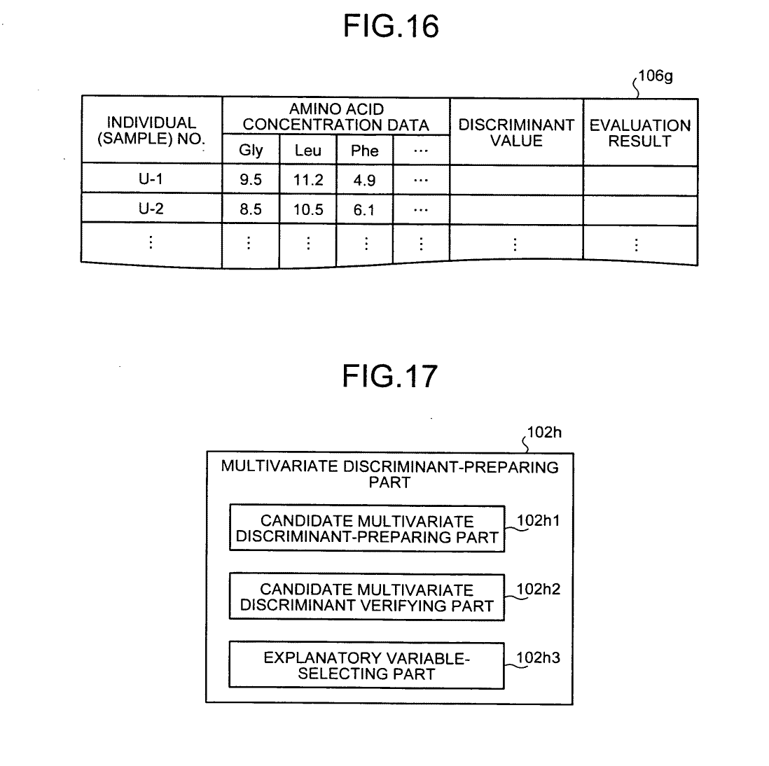

Returning to FIG. 6, the evaluation result file 106g stores the evaluation results obtained in the discriminant value criterion-evaluating part 102j described below (specifically the discrimination results obtained in a discriminant value criterion-discriminating part 102j1 described below). FIG. 16 is a chart showing an example of information stored in the evaluation result file 106g. The information stored in the evaluation result file 106g includes individual number for uniquely identifying the individual (sample) as the subject, previously obtained amino acid concentration data of the subject, discriminant value calculated in the multivariate discriminant, and evaluation result on the gastric cancer state (specifically, for example, discrimination result on the discrimination between the gastric cancer and the gastric cancer-free, discrimination result on the discrimination of the stage of gastric cancer, or discrimination result on the discrimination of the presence or absence of metastasis of gastric cancer to other organs) that are correlated to one another.

Returning to FIG. 6, the memory device 106 stores various Web data for providing the client apparatuses 200 with web site information, CGI programs, and others as information other than the information described above. The Web data include data for displaying various Web pages described below and others, and the data are generated as, for example, HTML (HyperText Markup Language) or XML (Extensible Markup Language) text file. Files for components and files for operation for generation of the Web data, other temporary files, and the like are also stored in the memory device 106. In addition, the memory device 106 may store as needed sound files of sounds for transmission to the client apparatuses 200 in WAVE format or AIFF (Audio Interchange File Format) format and image files of still images or motion pictures in JPEG (Joint Photographic Experts Group) format or MPEG2 (Moving Picture Experts Group phase 2) format.

The communication interface 104 allows communication between the gastric cancer-evaluating apparatus 100 and the network 300 (or communication apparatus such as a router). Thus, the communication interface 104 has a function to communicate data via a communication line with other terminals.

The input/output interface 108 is connected to the input device 112 and the output device 114. A monitor (including a home television), a speaker, or a printer may be used as the output device 114 (hereinafter, the output device 114 may be described as a monitor 114). A keyboard, a mouse, a microphone, or a monitor functioning as a pointing device together with a mouse may be used as the input device 112.

The control device 102 has an internal memory storing control programs such as OS (Operating System), programs for various processing procedures, and other needed data, and performs various information processings according to these programs. As shown in the figure, the control device 102 includes mainly a request-interpreting part 102a, a browsing processing part 102b, an authentication-processing part 102c, an electronic mail-generating part 102d, a Web page-generating part 102e, a receiving part 102f, the gastric cancer state information-designating part 102g, the multivariate discriminant-preparing part 102h, the discriminant value-calculating part 102i, the discriminant value criterion-evaluating part 102j, a result outputting part 102k and a sending part 102m. The control device 102 performs data processings such as removal of data including defective, removal of data including many outliers, and removal of explanatory variables for the defective-including data in the gastric cancer state information transmitted from the database apparatus 400 and in the amino acid concentration data transmitted from the client apparatus 200.

The request-interpreting part 102a interprets the requests transmitted from the client apparatus 200 or the database apparatus 400 and sends the requests to other parts in the control device 102 according to results of interpreting the requests. Upon receiving browsing requests for various screens transmitted from the client apparatus 200, the browsing processing part 102b generates and transmits web data for these screens. Upon receiving authentication requests transmitted from the client apparatus 200 or the database apparatus 400, the authentication-processing part 102c performs authentication. The electronic mail-generating part 102d generates electronic mails including various kinds of information. The Web page-generating part 102e generates Web pages for users to browse with the client apparatus 200.

The receiving part 102f receives, via the network 300, information (specifically, the amino acid concentration data, the gastric cancer state information, the multivariate discriminant etc.) transmitted from the client apparatus 200 or the database apparatus 400. The gastric cancer state information-designating part 102g designates objective gastric cancer state index data and objective amino acid concentration data in preparing the multivariate discriminant.

The multivariate discriminant-preparing part 102h generates the multivariate discriminants based on the gastric cancer state information received in the receiving part 102f and the gastric cancer state information designated in the gastric cancer state information-designating part 102g. Specifically, the multivariate discriminant-preparing part 102h prepares the multivariate discriminant by selecting the candidate multivariate discriminant to be used as the multivariate discriminant from a plurality of the candidate multivariate discriminants, based on verification results accumulated by repeating processings in the candidate multivariate discriminant-preparing part 102h1, the candidate multivariate discriminant-verifying part 102h2, and the explanatory variable-selecting part 102h3 from the gastric cancer state information.

If the multivariate discriminant is stored previously in a predetermined region of the memory device 106, the multivariate discriminant-preparing part 102h may generate the multivariate discriminant by selecting the desired multivariate discriminant out of the memory device 106. Alternatively, the multivariate discriminant-preparing part 102h may generate the multivariate discriminant by selecting and downloading the desired multivariate discriminant from the multivariate discriminants previously stored in another computer apparatus (e.g., the database apparatus 400).

Hereinafter, a configuration of the multivariate discriminant-preparing part 102h will be described with reference to FIG. 17. FIG. 17 is a block diagram showing the configuration of the multivariate discriminant-preparing part 102h, and only a part in the configuration related to the present invention is shown conceptually. The multivariate discriminant-preparing part 102h has the candidate multivariate discriminant-preparing part 102h1, the candidate multivariate discriminant-verifying part 102h2, and the explanatory variable-selecting part 102h3, additionally. The candidate multivariate discriminant-preparing part 102h1 prepares the candidate multivariate discriminant that is a candidate of the multivariate discriminant, from the gastric cancer state information based on a predetermined discriminant-preparing method. The candidate multivariate discriminant-preparing part 102h1 may prepare a plurality of the candidate multivariate discriminants from the gastric cancer state information, by using a plurality of the different discriminant-preparing methods. The candidate multivariate discriminant-verifying part 102h2 verifies the candidate multivariate discriminants prepared in the candidate multivariate discriminant-preparing part 102h1 based on a particular verifying method. The candidate multivariate discriminant-verifying part 102h2 may verify at least one of the discrimination rate, sensitivity, specificity, and information criterion of the candidate multivariate discriminants based on at least one of the bootstrap method, holdout method, and leave-one-out method. The explanatory variable-selecting part 102h3 selects the combination of the amino acid concentration data contained in the gastric cancer state information used in preparing the candidate multivariate discriminant, by selecting the explanatory variables of the candidate multivariate discriminant based on a particular explanatory variable-selecting method from the verification results obtained in the candidate multivariate discriminant-verifying part 102h2. The explanatory variable-selecting part 102h3 may select the explanatory variables of the candidate multivariate discriminant based on at least one of the stepwise method, best path method, local search method, and genetic algorithm from the verification results.

Returning to FIG. 6, the discriminant value-calculating part 102i calculates the discriminant value that is a value of the multivariate discriminant, based on (a) the concentration value of at least one of Asn, Cys, His, Met, Orn, Phe, Trp, Pro, Lys, Leu, Glu, Arg, Ala, Thr, and Tyr contained in the amino acid concentration data of the subject received in the receiving part 102f and (b) the multivariate discriminant containing at least one of Asn, Cys, His, Met, Orn, Phe, Trp, Pro, Lys, Leu, Glu, Arg, Ala, Thr, and Tyr as the explanatory variable prepared in the multivariate discriminant-preparing part 102h.

The multivariate discriminant may be expressed by one fractional expression or the sum of a plurality of the fractional expressions and may contain at least one of Asn, Cys, His, Met, Orn, Phe, Trp, Pro, Lys, Leu, Glu, Arg, Ala, Thr, and Tyr as the explanatory variable in any one of the numerator and denominator or both in the fractional expression constituting the multivariate discriminant. Specifically, (a) when the discrimination between the gastric cancer and the gastric cancer-free is conducted, the multivariate discriminant may be formula 1, 2, or 3, (b) when the discrimination of the stage of gastric cancer is conducted, the multivariate discriminant may be formula 4, and (c) when the discrimination of the presence or absence of metastasis of gastric cancer to other organs is conducted, the multivariate discriminant may be formula 5:

a₁×Orn/(Trp+His)+b₁×(ABA+Ile)/Leu+c₁ (formula 1)

a₂×Glu/His+b₂×Ser/Trp+c₂×Arg/Pro+d₂ (formula 2)

a₃×Trp/Gln+b₃×His/Glu+c₃ (formula 3)

a₄×Gly/(Glu+Trp+Val)+b₄×Arg/His+c₄ (formula 4)

a₅×Ile/Glu+b₅×(Gly+Asn+Arg)/His+c₅ (formula 5)

wherein a₁ and b₁ in the formula 1 are arbitrary non-zero real numbers, c₁ in the formula 1 is arbitrary real number, a₂, b₂, and c₂ in the formula 2 are arbitrary non-zero real numbers, d₂ in the formula 2 is arbitrary real number, a₃ and b₃ in the formula 3 are arbitrary non-zero real numbers, c₃ in the formula 3 is arbitrary real number, a₄ and b₄ in the formula 4 are arbitrary non-zero real numbers, c₄ in the formula 4 is arbitrary real number, a₅ and b₅ in the formula 5 are arbitrary non-zero real numbers, and c₅ in the formula 5 is arbitrary real number.

The multivariate discriminant may be any one of a logistic regression equation, a linear discriminant, a multiple regression equation, a discriminant prepared by a support vector machine, a discriminant prepared by a Mahalanobis' generalized distance method, a discriminant prepared by canonical discriminant analysis, and a discriminant prepared by a decision tree. Specifically, the multivariate discriminant may be the logistic regression equation with Orn, Gln, Trp, and Cit as the explanatory variables, the linear discriminant with Orn, Gln, Trp, Phe, Cit, and Try as the explanatory variables, the logistic regression equation with Glu, Phe, His, and Trp as the explanatory variables, the linear discriminant with Glu, Pro, His, and Trp as the explanatory variables, the logistic regression equation with Val, Ile, His, and Trp as the explanatory variables, or the linear discriminant with Thr, Ile, His, and Trp as the explanatory variables.

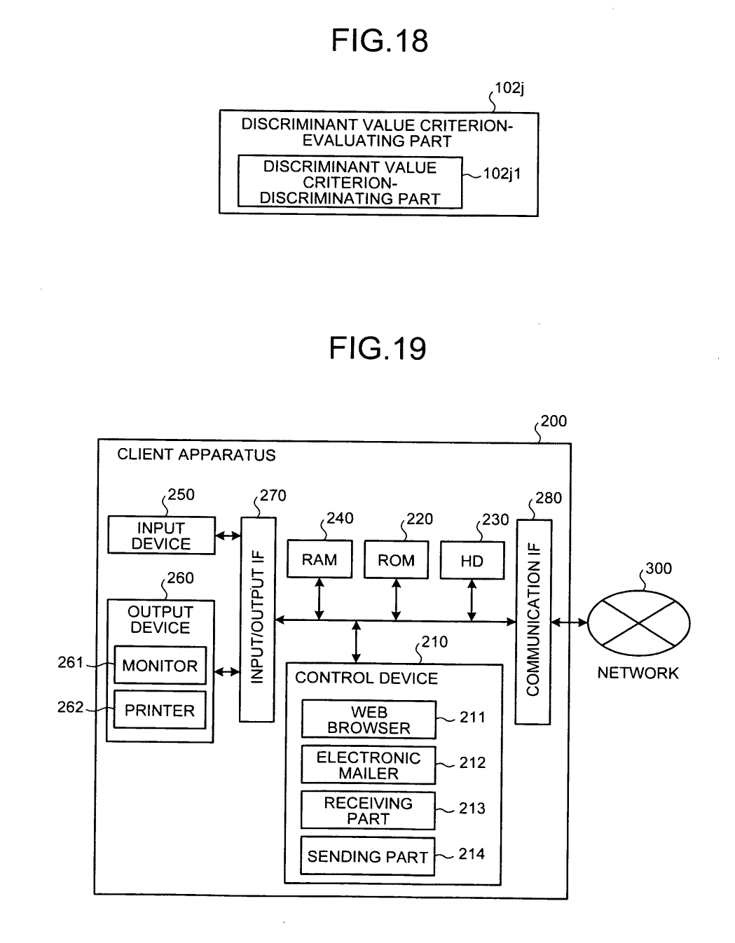

The discriminant value criterion-evaluating part 102j evaluates the gastric cancer state in the subject based on the discriminant value calculated in the discriminant value-calculating part 102i. The discriminant value criterion-evaluating part 102j further includes the discriminant value criterion-discriminating part 102j1. Now, a configuration of the discriminant value criterion-evaluating part 102j will be described with reference to FIG. 18. FIG. 18 is a block diagram showing the configuration of the discriminant value criterion-evaluating part 102j, and only a part in the configuration related to the present invention is shown conceptually. The discriminant value criterion-discriminating part 102j1 discriminates between the gastric cancer and the gastric cancer-free, discriminates the stage of gastric cancer, or discriminates the presence or absence of metastasis of gastric cancer to other organs in the subject based on the discriminant value. Specifically, the discriminant value criterion-discriminating part 102j1 compares the discriminant value with a predetermined threshold value (cutoff value), thereby discriminating between the gastric cancer and the gastric cancer-free, discriminating the stage of gastric cancer, or discriminating the presence or absence of metastasis of gastric cancer to other organs in the subject.

Returning to FIG. 6, the result outputting part 102k outputs, into the output device 114, the processing results in each processing part in the control device 102 (the evaluation results obtained in the discriminant value criterion-evaluating part 102j (specifically the discrimination results obtained in the discriminant value criterion-discriminating part 102j1)) etc.

The sending part 102m transmits the evaluation results to the client apparatus 200 that is a sender of the amino acid concentration data of the subject, and transmits the multivariate discriminants prepared in the gastric cancer-evaluating apparatus 100 and the evaluation results to the database apparatus 400.

Hereinafter, a configuration of the client apparatus 200 in the present system will be described with reference to FIG. 19. FIG. 19 is a block diagram showing an example of the configuration of the client apparatus 200 in the present system, and only the part in the configuration relevant to the present invention is shown conceptually. The client apparatus 200 includes a control device 210, ROM 220, HD (Hard Disk) 230, RAM 240, an input device 250, an output device 260, an input/output IF 270, and a communication IF 280 that are connected communicatively to one another through a communication channel.

The control device 210 has a Web browser 211, an electronic mailer 212, a receiving part 213, and a sending part 214. The Web browser 211 performs browsing processings of interpreting Web data and displaying the interpreted Web data on a monitor 261 described below. The Web browser 211 may have various plug-in softwares such as stream player having functions to receive, display and feedback streaming screen images. The electronic mailer 212 sends and receives electronic mails using a particular protocol (e.g., SMTP (Simple Mail Transfer Protocol) or POP3 (Post Office Protocol version 3)). The receiving part 213 receives various kinds of information such as the evaluation results transmitted from the gastric cancer-evaluating apparatus 100, via the communication IF 280. The sending part 214 sends various kinds of information such as the amino acid concentration data of the subject, via the communication IF 280, to the gastric cancer-evaluating apparatus 100.

The input device 250 is for example a keyboard, a mouse or a microphone. The monitor 261 described below also functions as a pointing device together with a mouse. The output device 260 is an output means for outputting information received via the communication IF 280, and includes the monitor 261 (including home television) and a printer 262. In addition, the output device 260 may have a speaker or the like additionally. The input/output IF 270 is connected to the input device 250 and the output device 260.

The communication IF 280 connects the client apparatus 200 to the network 300 (or communication apparatus such as a router) communicatively. In other words, the client apparatuses 200 are connected to the network 300 via a communication apparatus such as a modem, TA (Terminal Adapter) or a router, and a telephone line, or a private line. In this way, the client apparatuses 200 can access to the gastric cancer-evaluating apparatus 100 by using a particular protocol.

The client apparatus 200 may be realized by installing softwares (including programs, data and others) for a Web data-browsing function and an electronic mail-processing function to an information processing apparatus (for example, an information processing terminal such as a known personal computer, a workstation, a family computer, Internet TV (Television), PHS (Personal Handyphone System) terminal, a mobile phone terminal, a mobile unit communication terminal or PDA (Personal Digital Assistants)) connected as needed with peripheral devices such as a printer, a monitor, and an image scanner.

All or a part of processings of the control device 210 in the client apparatus 200 may be performed by CPU and programs read and executed by the CPU. Computer programs for giving instructions to the CPU and executing various processings together with the OS (Operating System) are recorded in the ROM 220 or HD 230. The computer programs, which are executed as they are loaded in the RAM 240, constitute the control device 210 with the CPU. The computer programs may be stored in application program servers connected via any network to the client apparatus 200, and the client apparatus 200 may download all or a part of them as needed. All or any part of processings of the control device 210 may be realized by hardware such as wired-logic.

Hereinafter, the network 300 in the present system will be described with reference to FIGS. 4 and 5. The network 300 has a function to connect the gastric cancer-evaluating apparatus 100, the client apparatuses 200, and the database apparatus 400 mutually, communicatively to one another, and is for example the Internet, an intranet, or LAN (Local Area Network (both wired/wireless)). The network 300 may be VAN (Value Added Network), a personal computer communication network, a public telephone network (including both analog and digital), a leased line network (including both analog and digital), CATV (Community Antenna Television) network, a portable switched network or a portable packet-switched network (including IMT2000 (International Mobile Telecommunication 2000) system, GSM (Global System for Mobile Communications) system, or PDC (Personal Digital Cellular)/PDC-P system), a wireless calling network, a local wireless network such as Bluetooth (registered trademark), PHS network, a satellite communication network (including CS (Communication Satellite), BS (Broadcasting Satellite), ISDB (Integrated Services Digital Broadcasting), and the like), or the like.

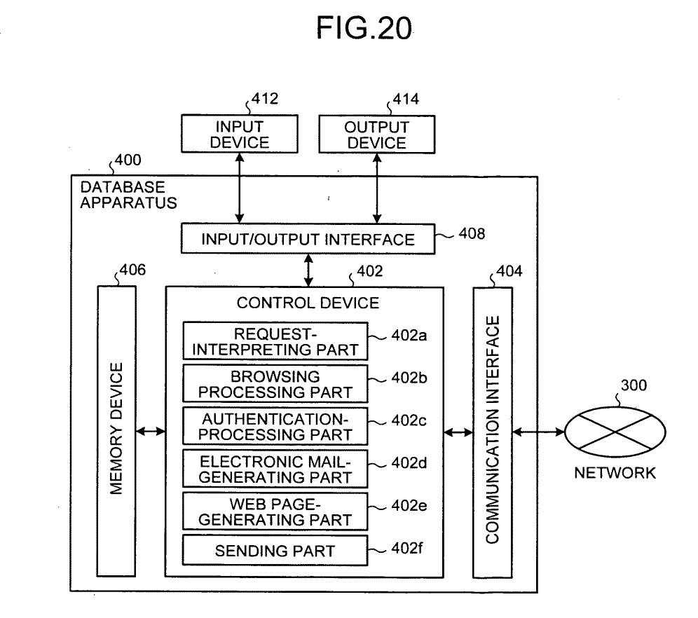

Hereinafter, the configuration of the database apparatus 400 in the present system will be described with reference to FIG. 20. FIG. 20 is a block diagram showing an example of the configuration of the database apparatus 400 in the present system, showing conceptually only the region relevant to the present invention.

The database apparatus 400 has functions to store, for example, the gastric cancer state information used in preparing the multivariate discriminants in the gastric cancer-evaluating apparatus 100 or in the database apparatus 400, the multivariate discriminants prepared in the gastric cancer-evaluating apparatus 100, and the evaluation results obtained in the gastric cancer-evaluating apparatus 100. As shown in FIG. 20, the database apparatus 400 includes (a) a control device 402, such as CPU, which integrally controls the entire database apparatus 400, (b) a communication interface 404 connecting the database apparatus to the network 300 communicatively via a communication apparatus such as a router and via wired or wireless communication circuits such as a private line, (c) a memory device 406 storing various databases, tables and files (for example, files for Web pages), and (d) an input/output interface 408 connected to an input device 412 and an output device 414, and these parts are connected communicatively to each other via any communication channel.

The memory device 406 is a storage means, and may be, for example, memory apparatus such as RAM or ROM, a fixed disk drive such as a hard disk, a flexible disk, an optical disk, and the like. The memory device 406 stores various processings used in various processings. The communication interface 404 allows communication between the database apparatus 400 and the network 300 (or a communication apparatus such as a router). Thus, the communication interface 404 has a function to communicate data via a communication line with other terminals. The input/output interface 408 is connected to the input device 412 and the output device 414. A monitor (including a home television), a speaker, or a printer may be used as the output device 414 (hereinafter, the output device 414 may be described as a monitor 414). A keyboard, a mouse, a microphone, or a monitor functioning as a pointing device together with a mouse may be used as the input device 412.

The control device 402 has an internal memory storing control programs such as OS (Operating System), programs for various processing procedures, and other needed data, and performs various information processings according to these programs. As shown in the figure, the control device 402 includes mainly a request-interpreting part 402a, a browsing processing part 402b, an authentication-processing part 402c, an electronic mail-generating part 402d, a Web page-generating part 402e, and a sending part 402f.

The request-interpreting part 402a interprets the request transmitted from the gastric cancer-evaluating apparatus 100 and sends the request to other parts in the control device 402 according to results of interpreting the requests. Upon receiving browsing requests for various screens transmitted from the gastric cancer-evaluating apparatus 100, the browsing processing part 402b generates and transmits web data for these screens. Upon receiving authentication requests transmitted from the gastric cancer-evaluating apparatus 100, the authentication-processing part 402c performs authentication. The electronic mail-generating part 402d generates electronic mails including various kinds of information. The Web page-generating part 402e generates Web pages for users to browse with the client apparatus 200. The sending part 402f transmits various kinds of information such as the gastric cancer state information and the multivariate discriminants to the gastric cancer-evaluating apparatus 100.

### 2-3. Processing in the Present System

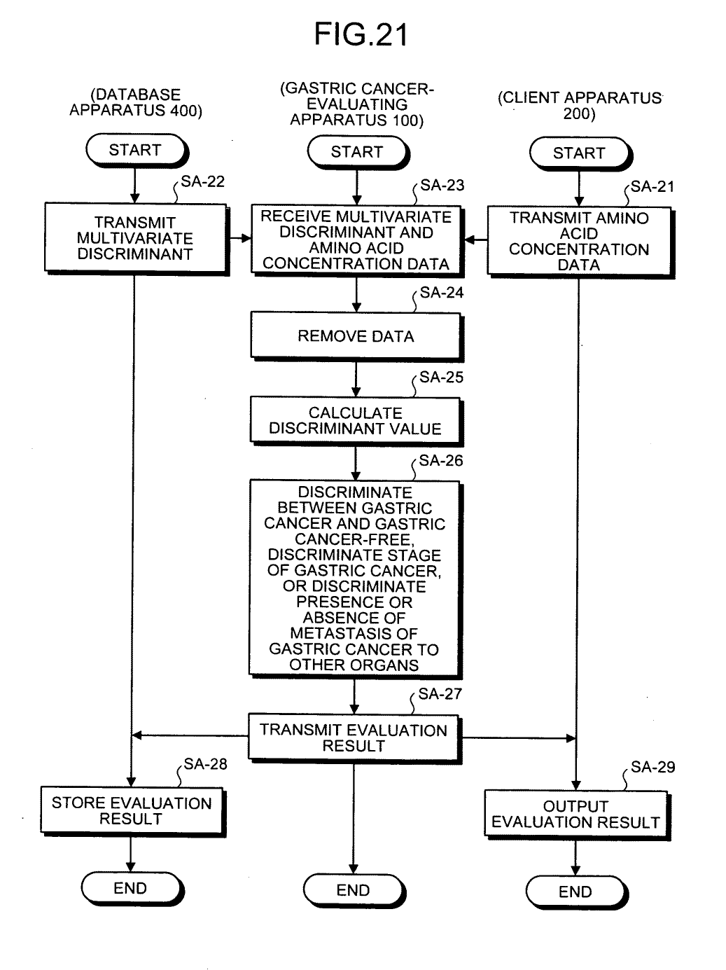

Here, an example of a gastric cancer evaluation service processing performed in the present system constituted as described above will be described with reference to FIG. 21. FIG. 21 is a flowchart showing the example of the gastric cancer evaluation service processing.

The amino acid concentration data used in the present processing is data concerning the concentration values of amino acids obtained by analyzing blood previously collected from an individual. Hereinafter, the method of analyzing blood amino acid will be described briefly.

First, a blood sample is collected in a heparin-treated tube, and then the blood plasma is separated by centrifugation of the tube. All blood plasma samples separated are frozen and stored at −70° C. before a measurement of an amino acid concentration. Before the measurement of the amino acid concentration, the blood plasma samples are deproteinized by adding sulfosalicylic acid to a concentration of 3%. An amino acid analyzer by high-performance liquid chromatography (HPLC) by using ninhydrin reaction in the post column is used for the measurement of the amino acid concentration.

First, the client apparatus 200 accesses the gastric cancer-evaluating apparatus 100 when the user specifies the Web site address (such as URL) provided from the gastric cancer-evaluating apparatus 100, via the input device 250 on the screen displaying the Web browser 211. Specifically, when the user instructs update of the Web browser 211 screen on the client apparatus 200, the Web browser 211 sends the Web site address provided from the gastric cancer-evaluating apparatus 100 by a particular protocol to the gastric cancer-evaluating apparatus 100, thereby transmitting requests demanding a transmission of Web page corresponding to an amino acid concentration data transmission screen to the gastric cancer-evaluating apparatus 100 based on a routing of the address.

Then, upon receipt of the request transmitted from the client apparatus 200, the request-interpreting part 102a in the gastric cancer-evaluating apparatus 100 analyzes the transmitted requests and sends the requests to other parts in the control device 102 according to analytical results. Specifically, when the transmitted requests are requests to send the Web page corresponding to the amino acid concentration data transmission screen, mainly the browsing processing part 102b in the gastric cancer-evaluating apparatus 100 obtains the Web data for displaying the Web page stored in a predetermined region of the memory device 106 and sends the obtained Web data to the client apparatus 200. More specifically, upon receiving the requests to transmit the Web page corresponding to the amino acid concentration data transmission screen by the user, the control device 102 in the gastric cancer-evaluating apparatus 100 demands inputs of user ID and user password from the user. If the user ID and password are input, the authentication-processing part 102c in the gastric cancer-evaluating apparatus 100 examines the input user ID and password by comparing them with the user ID and user password stored in the user information file 106a for authentication. Only when the user is authenticated, the browsing processing part 102b in the gastric cancer-evaluating apparatus 100 sends the Web data for displaying the Web page corresponding to the amino acid concentration data transmission screen to the client apparatus 200. The client apparatus 200 is identified with the IP (Internet Protocol) address transmitted from the client apparatus 200 together with the transmission requests.

Then, the client apparatus 200 receives, in the receiving part 213, the Web data (for displaying the Web page corresponding to the amino acid concentration data transmission screen) transmitted from the gastric cancer-evaluating apparatus 100, interprets the received Web data with the Web browser 211, and displays the amino acid concentration data transmission screen on the monitor 261.

When the user inputs and selects, via the input device 250, for example the amino acid concentration data of the individual on the amino acid concentration data transmission screen displayed on the monitor 261, the sending part 214 in the client apparatus 200 transmits an identifier for identifying input information and selected items to the gastric cancer-evaluating apparatus 100, thereby transmitting the amino acid concentration data of the individual as the subject to the gastric cancer-evaluating apparatus 100 (step SA-21). In the step SA-21, the transmission of the amino acid concentration data may be realized for example by using an existing file transfer technology such as FTP (File Transfer Protocol).

Then, the request-interpreting part 102a of the gastric cancer-evaluating apparatus 100 interprets the identifier transmitted from the client apparatus 200 thereby interpreting the requests from the client apparatus 200, and requests the database apparatus 400 to send the multivariate discriminants for the evaluation of gastric cancer (specifically, for example, for the discrimination of the 2 groups of the gastric cancer and the gastric cancer-free, for the discrimination of the stage of gastric cancer, or for the discrimination of the presence or absence of metastasis of gastric cancer to other organs).

Then, the request-interpreting part 402a in the database apparatus 400 interprets the transmission requests from the gastric cancer-evaluating apparatus 100 and transmits, to the gastric cancer-evaluating apparatus 100, the multivariate discriminant (for example, the updated newest multivariate discriminant) stored in a predetermined memory region of the memory device 406 containing at least one of Asn, Cys, His, Met, Orn, Phe, Trp, Pro, Lys, Leu, Glu, Arg, Ala, Thr, and Tyr as the explanatory variables (step SA-22).

In step SA-22, the multivariate discriminant transmitted to the gastric cancer-evaluating apparatus 100 may be expressed by one fractional expression or the sum of a plurality of the fractional expressions, and may contain at least one of Asn, Cys, His, Met, Orn, Phe, Trp, Pro, Lys, Leu, Glu, Arg, Ala, Thr, and Tyr as the explanatory variable in any one of the numerator and denominator or both in the fractional expression constituting the multivariate discriminant. Specifically, (a) when the discrimination between the gastric cancer and the gastric cancer-free is conducted in step SA-26, the multivariate discriminant transmitted to the gastric cancer-evaluating apparatus 100 may be formula 1, 2, or 3, (b) when the discrimination of the stage of gastric cancer is conducted in step SA-26, the multivariate discriminant may be formula 4, and (c) when the discrimination of the presence or absence of metastasis of gastric cancer to other organs is conducted in step SA-26, the multivariate discriminant may be formula 5:

a₁×Orn/(Trp+His)+b₁×(ABA+Ile)/Leu+c₁ (formula 1)

a₂×Glu/His+b₂×Ser/Trp+c₂×Arg/Pro+d₂ (formula 2)

a₃×Trp/Gln+b₃×His/Glu+c₃ (formula 3)

a₄×Gly/(Glu+Trp+Val)+b₄×Arg/His+c₄ (formula 4)

a₅×Ile/Glu+b₅×(Gly+Asn+Arg)/His+c₅ (formula 5)

wherein a₁ and b₁ in the formula 1 are arbitrary non-zero real numbers, c₁ in the formula 1 is arbitrary real number, a₂, b₂, and c₂ in the formula 2 are arbitrary non-zero real numbers, d₂ in the formula 2 is arbitrary real number, a₃ and b₃ in the formula 3 are arbitrary non-zero real numbers, c₃ in the formula 3 is arbitrary real number, a₄ and b₄ in the formula 4 are arbitrary non-zero real numbers, c₄ in the formula 4 is arbitrary real number, a₅ and b₅ in the formula 5 are arbitrary non-zero real numbers, and c₅ in the formula 5 is arbitrary real number.

In step SA-22, the multivariate discriminant transmitted to the gastric cancer-evaluating apparatus 100 may be any one of a logistic regression equation, a linear discriminant, a multiple regression equation, a discriminant prepared by a support vector machine, a discriminant prepared by a Mahalanobis' generalized distance method, a discriminant prepared by canonical discriminant analysis, and a discriminant prepared by a decision tree. Specifically, the multivariate discriminant transmitted to the gastric cancer-evaluating apparatus 100 may be the logistic regression equation with Orn, Gln, Trp, and Cit as the explanatory variables, the linear discriminant with Orn, Gln, Trp, Phe, Cit, and Try as the explanatory variables, the logistic regression equation with Glu, Phe, His, and Trp as the explanatory variables, the linear discriminant with Glu, Pro, His, and Trp as the explanatory variables, the logistic regression equation with Val, Ile, His, and Trp as the explanatory variables, or the linear discriminant with Thr, Ile, His, and Trp as the explanatory variables.

The gastric cancer-evaluating apparatus 100 receives, in the receiving part 102f, the amino acid concentration data of the individual transmitted from the client apparatuses 200 and the multivariate discriminant transmitted from the database apparatus 400, and stores the received amino acid concentration data in a predetermined memory region of the amino acid concentration data file 106b and the received multivariate discriminant in a predetermined memory region of the multivariate discriminant file 106e4 (step SA-23).

Then, the control device 102 in the gastric cancer-evaluating apparatus 100 removes data such as defective and outliers from the amino acid concentration data of the individual received in step SA-23 (step SA-24).

Then, the discriminant value in the discriminant value-calculating part 102i in the gastric cancer-evaluating apparatus 100 calculates the discriminant value based on both the multivariate discriminant received in step SA-23 and the amino acid concentration data of the individual from which the data such as the defective and outliers have been removed in step SA-24 (step SA-25).

Then, the discriminant value criterion-discriminating part 102j1 in the gastric cancer-evaluating apparatus 100 compares the discriminant value calculated in step SA-25 with a previously established threshold (cutoff value), thereby discriminating between the gastric cancer and the gastric cancer-free, discriminating the stage of gastric cancer, or discriminating the presence or absence of metastasis of gastric cancer to other organs in the individual, and the discrimination results are stored in a predetermined memory region of the evaluation result file 106g (step SA-26).

Then, the sending part 102m in the gastric cancer-evaluating apparatus 100 sends, to the client apparatus 200 that has sent the amino acid concentration data and to the database apparatus 400, the discrimination results (the discrimination results on the discrimination between the gastric cancer and the gastric cancer-free, the discrimination results on the discrimination of the stage of gastric cancer, or the discrimination results on the discrimination of the presence or absence of metastasis of gastric cancer to other organs) obtained in step SA-26 (step SA-27). Specifically, the gastric cancer-evaluating apparatus 100 first generates a Web page for displaying the discrimination results in the Web page-generating part 102e and stores the Web data corresponding to the generated Web page in a predetermined memory region of the memory device 106. Then, the user is authenticated as described above by inputting a predetermined URL (Uniform Resource Locator) into the Web browser 211 of the client apparatus 200 via the input device 250, and the client apparatus 200 sends a Web page browsing request to the gastric cancer-evaluating apparatus 100. The gastric cancer-evaluating apparatus 100 then interprets the browsing request transmitted from the client apparatus 200 in the browsing processing part 102b and reads the Web data corresponding to the Web page for displaying the discrimination results, out of the predetermined memory region of the memory device 106. The sending part 102m in the gastric cancer-evaluating apparatus 100 then sends the read-out Web data to the client apparatus 200 and simultaneously sends the Web data or the discrimination results to the database apparatus 400.

In step SA-27, the control device 102 in the gastric cancer-evaluating apparatus 100 may notify the discrimination results to the user client apparatus 200 by electronic mail. Specifically, the electronic mail-generating part 102d in the gastric cancer-evaluating apparatus 100 first acquires the user electronic mail address by referencing the user information stored in the user information file 106a based on the user ID and the like at the transmission timing. The electronic mail-generating part 102d in the gastric cancer-evaluating apparatus 100 then generates electronic mail data with the acquired electronic mail address as its mail address, including the user name and the discrimination results. The sending part 102m in the gastric cancer-evaluating apparatus 100 then sends the generated electronic mail data to the user client apparatus 200.

Also in step SA-27, the gastric cancer-evaluating apparatus 100 may send the discrimination results to the user client apparatus 200 by using, for example, an existing file transfer technology such as FTP.

Returning to FIG. 21, the control device 402 in the database apparatus 400 receives the discrimination results or the Web data transmitted from the gastric cancer-evaluating apparatus 100 and stores (accumulates) the received discrimination results or the received Web data in a predetermined memory region of the memory device 406 (step SA-28).

The receiving part 213 of the client apparatus 200 receives the Web data transmitted from the gastric cancer-evaluating apparatus 100, and the received Web data is interpreted with the Web browser 211, to display on the monitor 261 the Web page screen displaying the discrimination results of the individual (step SA-29). When the discrimination results are sent from the gastric cancer-evaluating apparatus 100 by electronic mail, the electronic mail transmitted from the gastric cancer-evaluating apparatus 100 is received at any timing, and the received electronic mail is displayed on the monitor 261 with the known function of the electronic mailer 212 in the client apparatus 200.

In this way, the user can confirm the discrimination results on the discrimination of the 2 groups of the gastric cancer and the gastric cancer-free in the individual, the discrimination results on the discrimination of the stage of gastric cancer in the individual, or the discrimination results on the discrimination between the 2 groups of the presence of metastasis of gastric cancer to other organs and the absence of the metastasis in the individual by browsing the Web page displayed on the monitor 261. The user can print out the content of the Web page displayed on the monitor 261 by the printer 262.

When the discrimination results are transmitted by electronic mail from the gastric cancer-evaluating apparatus 100, the user reads the electronic mail displayed on the monitor 261, whereby the user can confirm the discrimination results on the discrimination of the 2 groups of the gastric cancer and the gastric cancer-free in the individual, the discrimination results on the discrimination of the stage of gastric cancer in the individual, or the discrimination results on the discrimination between the 2 groups of the presence of metastasis of gastric cancer to other organs and the absence of the metastasis in the individual. The user may print out the content of the electronic mail displayed on the monitor 261 by the printer 262.

Given the foregoing description, the explanation of the gastric cancer evaluation service processing is finished.

### 2-4. Summary of the Second Embodiment and Other Embodiments

According to the gastric cancer-evaluating system described above in detail, the client apparatus 200 sends the amino acid concentration data of the individual to the gastric cancer-evaluating apparatus 100. Upon receiving the requests from the gastric cancer-evaluating apparatus 100, the database apparatus 400 transmits the multivariate discriminant for the evaluation of gastric cancer (specifically, for example, the multivariate discriminant for the discrimination between the 2 groups of the gastric cancer and the gastric cancer-free, the multivariate discriminant for the discrimination of the stage of gastric cancer, or the multivariate discriminant for the discrimination of the presence of metastasis of gastric cancer to other organs and the absence of the metastasis) to the gastric cancer-evaluating apparatus 100. By the gastric cancer-evaluating apparatus 100, (1) the amino acid concentration data transmitted from the client apparatus 200 is received and the multivariate discriminant transmitted from the database apparatus 400 is received simultaneously, (2) the discriminant values are calculated based on the received amino acid concentration data and the received multivariate discriminant, (3) the calculated discriminant values are compared with the previously established threshold, thereby discriminating between the gastric cancer and the gastric cancer-free, discriminating the stage of gastric cancer, or discriminating the presence or absence of metastasis of gastric cancer to other organs in the individual, and (4) the discrimination results are transmitted to the client apparatus 200 and the database apparatus 400. Then, the client apparatus 200 receives and displays the discrimination results transmitted from the gastric cancer-evaluating apparatus 100, and the database apparatus 400 receives and stores the discrimination results transmitted from the gastric cancer-evaluating apparatus 100. Thus, discriminant values obtained in multivariate discriminants useful for discriminating between the 2 groups of the gastric cancer and the gastric cancer-free, discriminating the stage of gastric cancer, or discriminating between the 2 groups of the presence of metastasis of gastric cancer to other organs and the absence of the metastasis can be utilized to bring about an effect of enabling accurately these discriminations.

According to the gastric cancer-evaluating system, the multivariate discriminant may be expressed by one fractional expression or the sum of a plurality of the fractional expressions and may contain at least one of Asn, Cys, His, Met, Orn, Phe, Trp, Pro, Lys, Leu, Glu, Arg, Ala, Thr, and Tyr as the explanatory variable in any one of the numerator and denominator or both in the fractional expression constituting the multivariate discriminant. Specifically, (a) when the discrimination between the gastric cancer and the gastric cancer-free is conducted, the multivariate discriminant may be formula 1, 2, or 3, (b) when the discrimination of the stage of gastric cancer is conducted, the multivariate discriminant may be formula 4, and (c) when the discrimination of the presence or absence of metastasis of gastric cancer to other organs is conducted, the multivariate discriminant may be formula 5:

a₁×Orn/(Trp+His)+b₁×(ABA+Ile)/Leu+c₁ (formula 1)

a₂×Glu/His+b₂×Ser/Trp+c₂×Arg/Pro+d₂ (formula 2)

a₃×Trp/Gln+b₃×His/Glu+c₃ (formula 3)

a₄×Gly/(Glu+Trp+Val)+b₄×Arg/His+c₄ (formula 4)

a₅×Ile/Glu+b₅×(Gly+Asn+Arg)/His+c₅ (formula 5)

wherein a₁ and b₁ in the formula 1 are arbitrary non-zero real numbers, c₁ in the formula 1 is arbitrary real number, a₂, b₂, and c₂ in the formula 2 are arbitrary non-zero real numbers, d₂ in the formula 2 is arbitrary real number, a₃ and b₃ in the formula 3 are arbitrary non-zero real numbers, c₃ in the formula 3 is arbitrary real number, a₄ and b₄ in the formula 4 are arbitrary non-zero real numbers, c₄ in the formula 4 is arbitrary real number, a₅ and b₅ in the formula 5 are arbitrary non-zero real numbers, and c₅ in the formula 5 is arbitrary real number. Thus, discriminant values obtained in multivariate discriminants useful particularly for discriminating between the 2 groups of the gastric cancer and the gastric cancer-free, discriminating the stage of gastric cancer, or discriminating between the 2 groups of the presence of metastasis of gastric cancer to other organs and the absence of the metastasis can be utilized to bring about an effect of enabling more accurately these discriminations. The multivariate discriminants described above can be prepared by a method described in International Publication WO 2004/052191 Pamphlet that is an international application filed by the present applicant or by a method (multivariate discriminant-preparing processing described later) described in International Publication WO 2006/098192 Pamphlet that is an international application filed by the present applicant. Any multivariate discriminants obtained by these methods can be preferably used in the evaluation of the gastric cancer state, regardless of the unit of the amino acid concentration in the amino acid concentration data as input data.

According to the gastric cancer-evaluating system, the multivariate discriminant may be any one of a logistic regression equation, a linear discriminant, a multiple regression equation, a discriminant prepared by a support vector machine, a discriminant prepared by a Mahalanobis' generalized distance method, a discriminant prepared by canonical discriminant analysis, and a discriminant prepared by a decision tree. Specifically, the multivariate discriminant may be the logistic regression equation with Orn, Gln, Trp, and Cit as the explanatory variables, the linear discriminant with Orn, Gln, Trp, Phe, Cit, and Try as the explanatory variables, the logistic regression equation with Glu, Phe, His, and Trp as the explanatory variables, the linear discriminant with Glu, Pro, His, and Trp as the explanatory variables, the logistic regression equation with Val, Ile, His, and Trp as the explanatory variables, or the linear discriminant with Thr, Ile, His, and Trp as the explanatory variables. Thus, discriminant values obtained in multivariate discriminants useful particularly for discriminating between the 2 groups of the gastric cancer and the gastric cancer-free, discriminating the stage of gastric cancer, or discriminating between the 2 groups of the presence of metastasis of gastric cancer to other organs and the absence of the metastasis can be utilized to bring about an effect of enabling more accurately these discriminations. The multivariate discriminants described above can be prepared by a method (multivariate discriminant-preparing processing described later) described in International Publication WO 2006/098192 Pamphlet that is an international application filed by the present applicant.

In addition to the second embodiment described above, the gastric cancer-evaluating apparatus, the gastric cancer-evaluating method, the gastric cancer-evaluating system, the gastric cancer-evaluating program product and the recording medium according to the present invention can be practiced in various different embodiments within the technological scope of the claims. For example, among the processings described in the second embodiment above, all or a part of the processings described above as performed automatically may be performed manually, and all or a part of the manually conducted processings may be performed automatically by known methods. In addition, the processing procedure, control procedure, specific name, various registered data, information including parameters such as retrieval condition, screen, and database configuration shown in the description above or drawings may be modified arbitrarily, unless specified otherwise. For example, the components of the gastric cancer-evaluating apparatus 100 shown in the figures are conceptual and functional and may not be the same physically as those shown in the figure. In addition, all or an arbitrary part of the operational function of each component and each device in the gastric cancer-evaluating apparatus 100 (in particular, the operational functions executed in the control device 102) may be executed by the CPU (Central Processing Unit) or the programs executed by the CPU, and may be realized as wired-logic hardware.

The “program” is a data processing method written in any language or by any description method and may be of any format such as source code or binary code. The “program” may not be limited to a program configured singly, and may include a program configured decentrally as a plurality of modules or libraries, and a program to achieve the function together with a different program such as OS (Operating System). The program is stored on a recording medium and read mechanically as needed by the gastric cancer-evaluating apparatus 100. Any well-known configuration or procedure may be used as specific configuration, reading procedure, installation procedure after reading, and the like for reading the programs recorded on the recording medium in each apparatus.

The “recording media” includes any “portable physical media”, “fixed physical media”, and “communication media”. Examples of the “portable physical media” include flexible disk, magnetic optical disk, ROM, EPROM (Erasable Programmable Read Only Memory), EEPROM (Electronically Erasable and Programmable Read Only Memory), CD-ROM (Compact Disk Read Only Memory), MO (Magneto-Optical disk), DVD (Digital Versatile Disk), and the like. Examples of the “fixed physical media” include ROM, RAM, HD, and the like which are installed in various computer systems. The “communication media” for example stores the program for a short period of time such as communication line and carrier wave when the program is transmitted via a network such as LAN (Local Area Network), WAN (Wide Area Network), or the Internet.

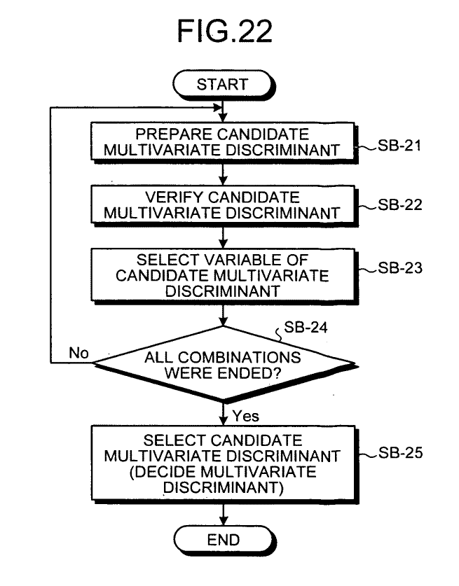

Finally, an example of the multivariate discriminant-preparing processing performed in the gastric cancer-evaluating apparatus 100 is described in detail with reference to FIG. 22. FIG. 22 is a flowchart showing an example of the multivariate discriminant-preparing processing. The multivariate discriminant-preparing processing may be performed in the database apparatus 400 handling the gastric cancer state information.

In the present description, the gastric cancer-evaluating apparatus 100 stores the gastric cancer state information previously obtained from the database apparatus 400 in a predetermined memory region of the gastric cancer state information file 106c. The gastric cancer-evaluating apparatus 100 shall store, in a predetermined memory region of the designated gastric cancer state information file 106d, the gastric cancer state information including the gastric cancer state index data and amino acid concentration data designated previously in the gastric cancer state information-designating part 102g.

The candidate multivariate discriminant-preparing part 102h1 in the multivariate discriminant-preparing part 102h first prepares the candidate multivariate discriminant according to a predetermined discriminant-preparing method from the gastric cancer state information stored in a predetermine memory region of the designated gastric cancer state information file 106d, and stores the prepared candidate multivariate discriminate in a predetermined memory region of the candidate multivariate discriminant file 106e1 (step SB-21). Specifically, the candidate multivariate discriminant-preparing part 102h1 in the multivariate discriminant-preparing part 102h first selects a desired method out of a plurality of different discriminant-preparing methods (including those for multivariate analysis such as principal component analysis, discriminant analysis, support vector machine, multiple regression analysis, logistic regression analysis, k-means method, cluster analysis, and decision tree) and determines the form of the candidate multivariate discriminant to be prepared based on the selected discriminant-preparing method. The candidate multivariate discriminant-preparing part 102h1 in the multivariate discriminant-preparing part 102h then performs various calculation corresponding to the selected function-selecting method (e.g., average or variance), based on the gastric cancer state information. The candidate multivariate discriminant-preparing part 102h1 in the multivariate discriminant-preparing part 102h then determines the parameters for the calculation result and the determined candidate multivariate discriminant. In this way, the candidate multivariate discriminant is generated based on the selected discriminant-preparing method. When candidate multivariate discriminants are generated simultaneously and concurrently (in parallel) by using a plurality of different discriminant-preparing methods in combination, the processings described above may be executed concurrently for each selected discriminant-preparing method. Alternatively when candidate multivariate discriminants are generated in series by using a plurality of different discriminant-preparing methods in combination, for example, candidate multivariate discriminants may be generated by converting the gastric cancer state information with the candidate multivariate discriminant prepared by performing principal component analysis and performing discriminant analysis of the converted gastric cancer state information.

The candidate multivariate discriminant-verifying part 102h2 in the multivariate discriminant-preparing part 102h verifies (mutually verifies) the candidate multivariate discriminant prepared in the step SB-21 according to a particular verifying method and stores the verification result in a predetermined memory region of the verification result file 106e2 (step SB-22). Specifically, the candidate multivariate discriminant-verifying part 102h2 in the multivariate discriminant-preparing part 102h first generates the verification data to be used in verification of the candidate multivariate discriminant, based on the gastric cancer state information stored in a predetermined memory region of the designated gastric cancer state information file 106d, and verifies the candidate multivariate discriminant according to the generated verification data. If a plurality of candidate multivariate discriminants is generated by using a plurality of different discriminant-preparing methods in the step SB-21, the candidate multivariate discriminant-verifying part 102h2 in the multivariate discriminant-preparing part 102h verifies each candidate multivariate discriminant corresponding to each discriminant-preparing method according to a particular verifying method. Here in the step SB-22, at least one of the discrimination rate, sensitivity, specificity, information criterion, and the like of the candidate multivariate discriminant may be verified based on at least one method of the bootstrap method, holdout method, leave-one-out method, and the like. Thus, it is possible to select the candidate multivariate discriminant higher in predictability or reliability, by taking the gastric cancer state information and diagnostic condition into consideration.

Then, the explanatory variable-selecting part 102h3 in the multivariate discriminant-preparing part 102h selects the combination of the amino acid concentration data contained in the gastric cancer state information used in preparing the candidate multivariate discriminant by selecting the explanatory variable of the candidate multivariate discriminant from the verification result obtained in the step SB-22 according to a predetermined explanatory variable-selecting method, and stores the gastric cancer state information including the selected combination of the amino acid concentration data in a predetermined memory region of the selected gastric cancer state information file 106e3 (step SB-23). When a plurality of candidate multivariate discriminants is generated by using a plurality of different discriminant-preparing methods in the step SB-21 and each candidate multivariate discriminant corresponding to each discriminant-preparing method is verified according to a predetermined verifying method in the step SB-22, the explanatory variable-selecting part 102h3 in the multivariate discriminant-preparing part 102h selects the explanatory variable of the candidate multivariate discriminant for each candidate multivariate discriminant corresponding to the verification result obtained in the step SB-22, according to a predetermined explanatory variable-selecting method in the step SB-23. Here in the step SB-23, the explanatory variable of the candidate multivariate discriminant may be selected from the verification results according to at least one of the stepwise method, best path method, local search method, and genetic algorithm. The best path method is a method of selecting an explanatory variable by optimizing an evaluation index of the candidate multivariate discriminant while eliminating the explanatory variables contained in the candidate multivariate discriminant one by one. In the step SB-23, the explanatory variable-selecting part 102h3 in the multivariate discriminant-preparing part 102h may select the combination of the amino acid concentration data based on the gastric cancer state information stored in a predetermined memory region of the designated gastric cancer state information file 106d.

The multivariate discriminant-preparing part 102h then judges whether all combinations of the amino acid concentration data contained in the gastric cancer state information stored in a predetermined memory region of the designated gastric cancer state information file 106d are processed, and if the judgment result is “End” (Yes in step SB-24), the processing advances to the next step (step SB-25), and if the judgment result is not “End” (No in step SB-24), it returns to the step SB-21. The multivariate discriminant-preparing part 102h judges whether the processing is performed a predetermined number of times, and if the judgment result is “End” (Yes in step SB-24), the processing may advance to the next step (step SB-25), and if the judgment result is not “End” (No in step SB-24), it may return to the step SB-21. The multivariate discriminant-preparing part 102h may judge whether the combination of the amino acid concentration data selected in the step SB-23 is the same as the combination of the amino acid concentration data contained in the gastric cancer state information stored in a predetermined memory region of the designated gastric cancer state information file 106d or the combination of the amino acid concentration data selected in the previous step SB-23, and if the judgment result is “the same” (Yes in step SB-24), the processing may advance to the next step (step SB-25) and if the judgment result is not “the same” (No in step SB-24), it may return to the step SB-21. If the verification result is specifically the evaluation value for each multivariate discriminant, the multivariate discriminant-preparing part 102h may advance to the step SB-25 or return to the step SB-21, based on the comparison of the evaluation value with a particular threshold corresponding to each discriminant-preparing method.

Then, the multivariate discriminant-preparing part 102h determines the multivariate discriminant by selecting the candidate multivariate discriminant used as the multivariate discriminant based on the verification results from a plurality of the candidate multivariate discriminants, and stores the determined multivariate discriminant (the selected candidate multivariate discriminant) in particular memory region of the multivariate discriminant file 106e4 (step SB-25). Here, in the step SB-25, for example, there are cases where the optimal multivariate discriminant is selected from the candidate multivariate discriminants prepared in the same discriminant-preparing method or the optical multivariate discriminant is selected from all candidate multivariate discriminants.

Given the foregoing description, the explanation of the multivariate discriminant-preparing processing is finished.

### Example 1

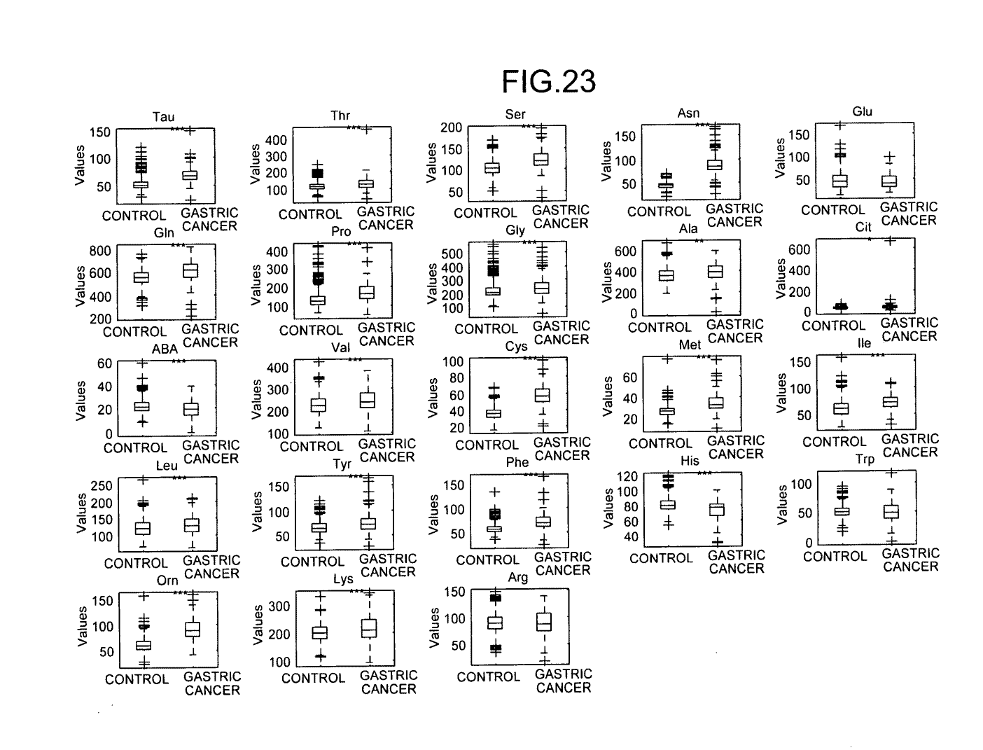

Blood samples of a group of gastric cancer patients definitively diagnosed as gastric cancer, and blood samples of a group of gastric cancer-free, are subjected to measurement of amino acid concentration in blood by the amino acid analysis method. The unit of amino acid concentration is nmol/ml. FIG. 23 is a boxplot showing the distribution of amino acid explanatory variables in the gastric cancer patients and the gastric cancer-free subjects. In FIG. 23, the horizontal axis indicates the gastric cancer-free group (control) and the gastric cancer group, and ABA and Cys in the figure represent α-ABA (α-aminobutyric acid) and Cystine, respectively. For the purpose of discrimination between the gastric cancer group and the gastric cancer-free group, a t-test between the 2 groups is performed.

In the gastric cancer group as compared with the gastric cancer-free group, Thr, Ser, Pro, Gly, Ala, Cit, Cys, Val, Met, Ile, Leu, Tyr, Phe, Orn, and Lys significantly increase (significant difference probability P<0.05), and ABA and His significantly decrease (significant difference probability P<0.05). Thus, it is made clear that the amino acid explanatory variables Thr, Ser, Pro, Gly, Ala, Cit, Cys, Val, Met, Ile, Leu, Tyr, Phe, Orn, Lys, ABA, and His have an ability to discriminate between the 2 groups of the gastric cancer group and the gastric cancer-free group.

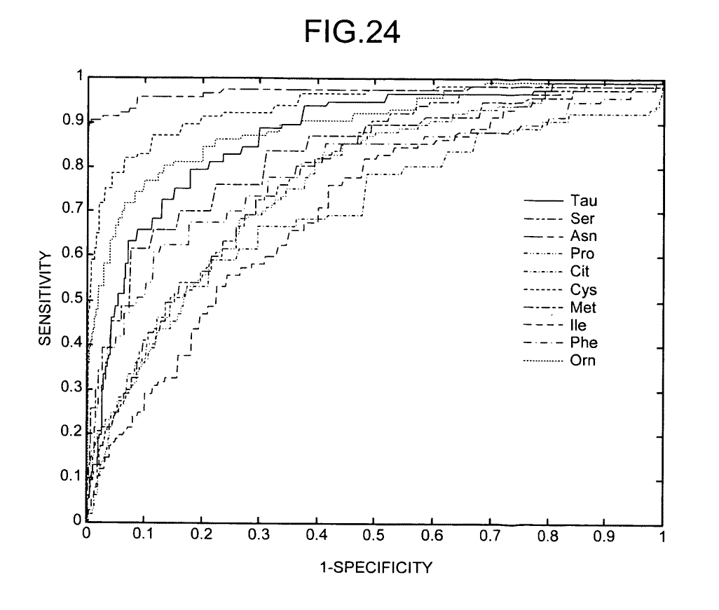

Furthermore, an evaluation using the area under the curve (AUC) of the ROC (receiver operating characteristic) curve (FIG. 24) is carried out for the discrimination between the 2 groups of the gastric cancer group and the gastric cancer-free group based on respective amino acid explanatory variables, and the AUC shows values larger than 0.7 for amino acid explanatory variables Ser, Asn, Pro, Cit, Cys, Met, Ile, Phe, His, and Orn. Therefore, it is made clear that the amino acid explanatory variables Ser, Asn, Cys, Pro, Cit, Met, Ile, Phe, His, and Orn have an ability to discriminate between the 2 groups of the gastric cancer group and the gastric cancer-free group.

### Example 2

The sample data used in Example 1 is used. Using a method described in International Publication WO 2004/052191 Pamphlet that is an international application filed by the present applicant, indices by which the performance of discriminating between the 2 groups of the gastric cancer group and the gastric cancer-free group is maximized with regard to the discrimination of gastric cancer are eagerly searched, and an index formula 1 is obtained among a plurality of indices having an equivalent performance.

(Asn)/(ABA)+(Leu)/(His) Index formula 1

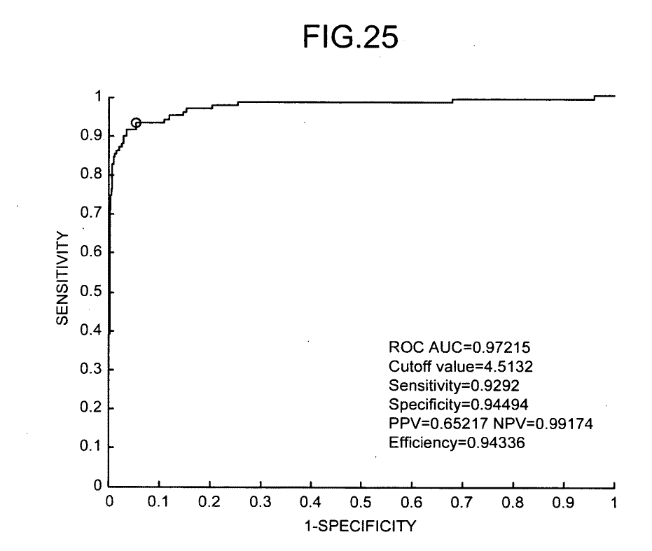

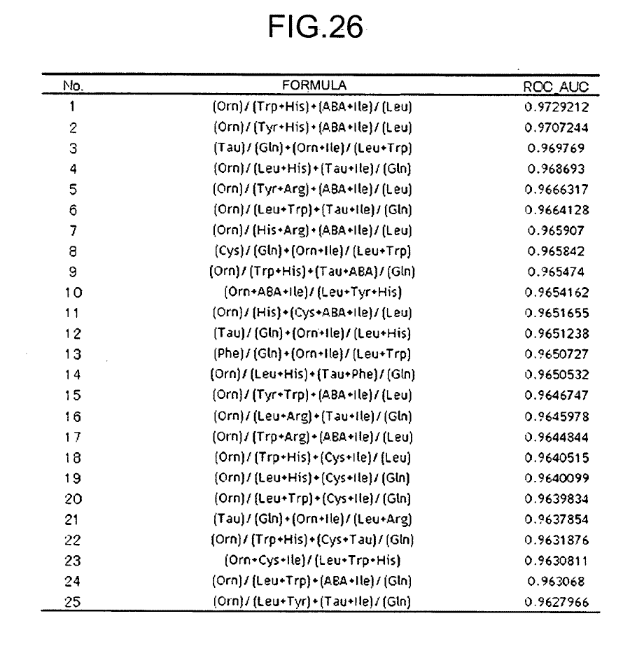

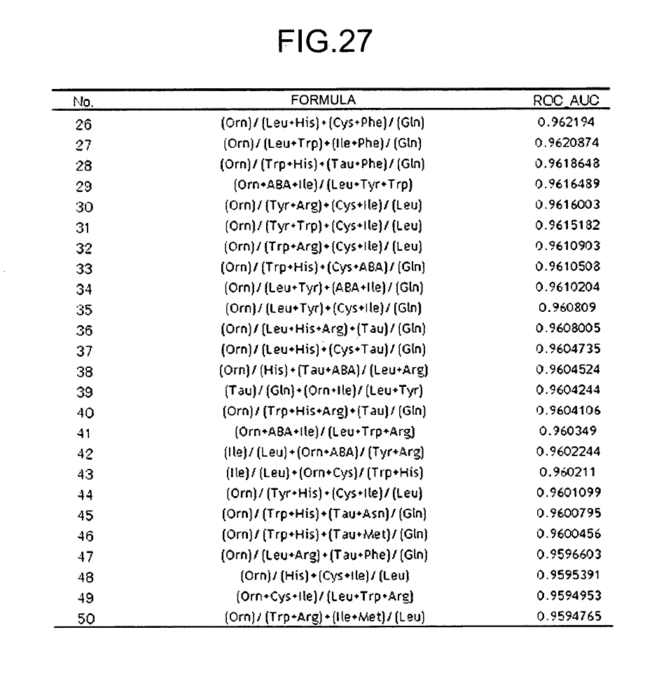

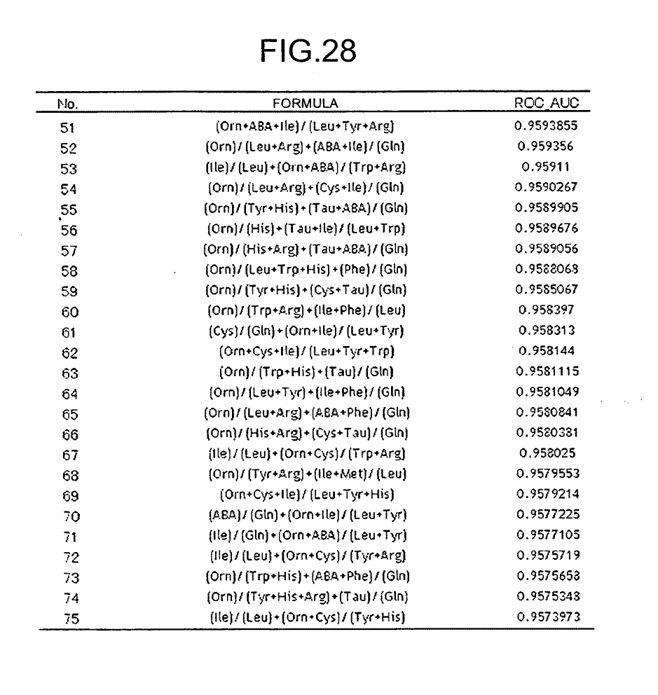

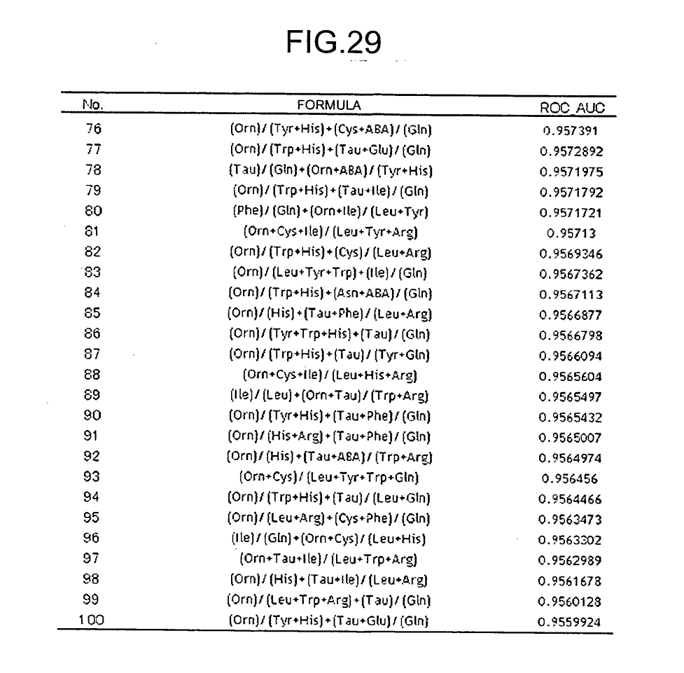

The performance for diagnosis of gastric cancer based on the index formula 1 is evaluated based on the AUC of the ROC curve (FIG. 25) in connection with the discrimination between the 2 groups of the gastric cancer group and the gastric cancer-free group, and an AUC of 0.972±0.011 (95% confidence interval: 0.951 to 0.994) is obtained. When the optimum cutoff value for the discrimination between the 2 groups of the gastric cancer group and the gastric cancer-free group by the index formula 1 is determined assuming that the symptom prevalence of the gastric cancer group is 0.038, the cutoff value is 4.51, and a sensitivity of 93%, a specificity of 94%, a positive predictive value of 65%, a negative predictive value of 99%, and a correct diagnostic rate of 94% are obtained. Thus, the index formula 1 is found to be a useful index with high diagnostic performance. In addition to that, a plurality of fractional expressions having a discrimination performance equivalent to that of the index formula 1 is obtained. Those fractional expressions are presented in FIG. 26, FIG. 27, FIG. 28 and FIG. 29.

### Example 3

The sample data used in Example 1 is used. Indices by which the performance of discriminating between the 2 groups of the gastric cancer group and the gastric cancer-free group is maximized with regard to gastric cancer are searched by logistic analysis (explanatory variable coverage method based on the BIC (bayesian information criterion) minimum criterion), and a logistic regression equation composed of Asn, Orn, Phe, and His (the numerical coefficients of the amino acid explanatory variables Asn, Orn, Phe, and His, and the constant terms are, in the same order, 0.291±0.051, 0.088±0.028, 0.116±0.025, −0.299±0.067, and −9.499±3.204, respectively) is obtained as an index formula 2.

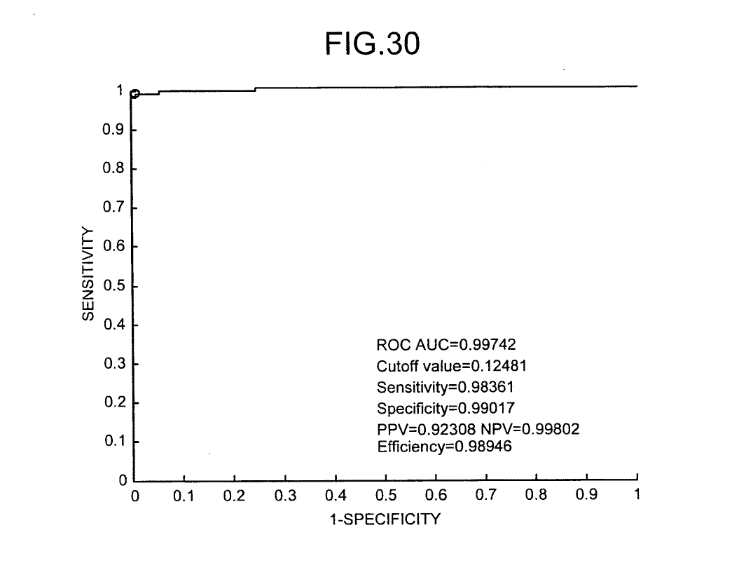

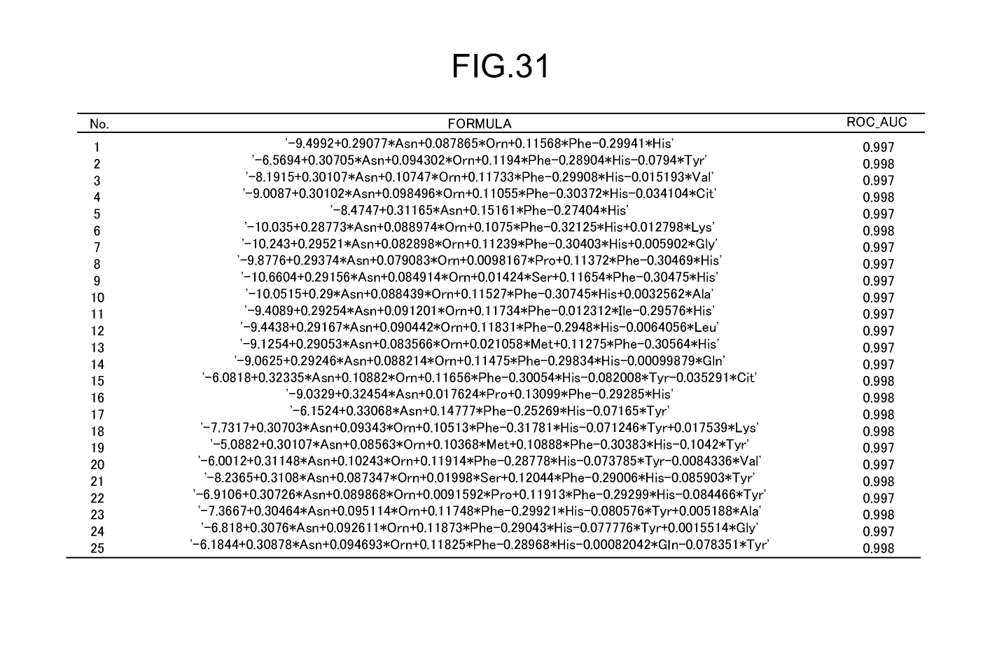

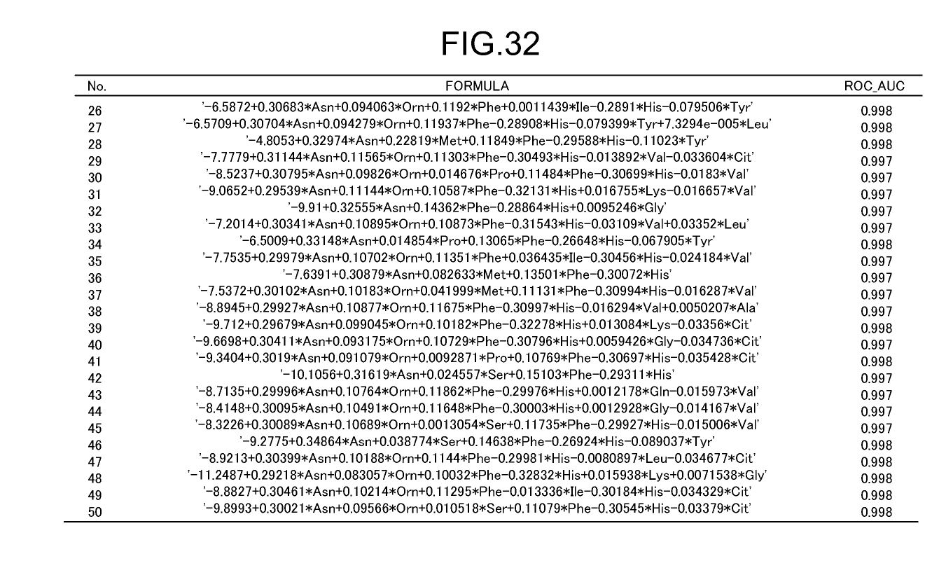

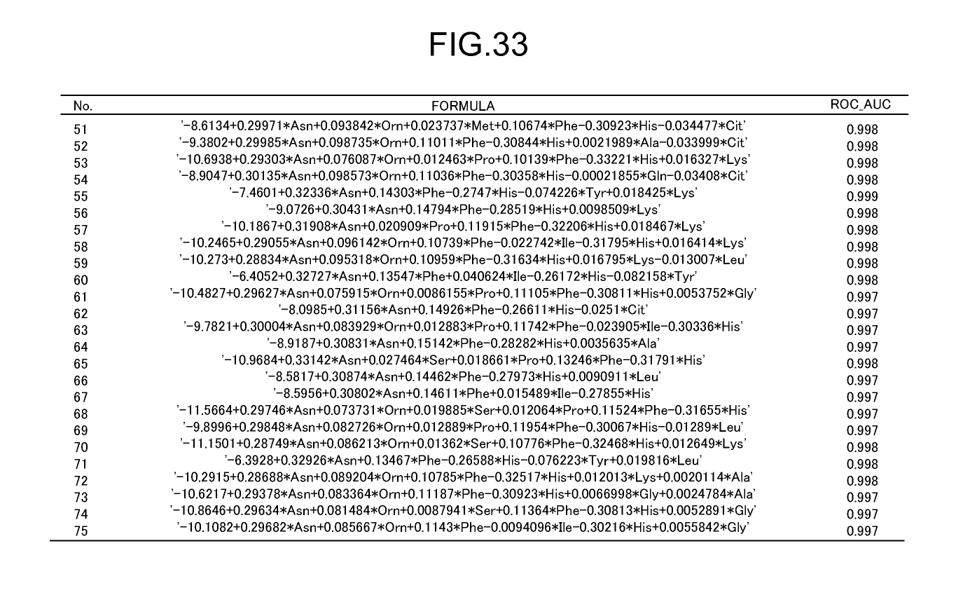

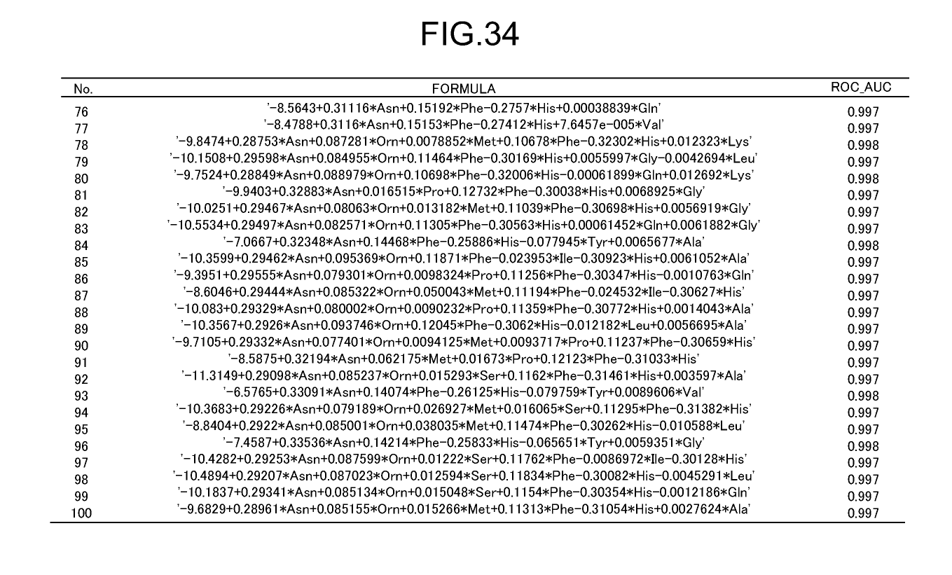

The performance for diagnosis of gastric cancer based on the index formula 2 is evaluated based on the AUC of the ROC curve (FIG. 30) in connection with the discrimination between the 2 groups of the gastric cancer group and the gastric cancer-free group, and an AUC of 0.997±0.002 (95% confidence interval: 0.993 to 1.00) is obtained. Thus, the index formula 2 is found to be a useful index with high diagnostic performance. When the optimum cutoff value for the discrimination between the 2 groups of the gastric cancer group and the gastric cancer-free group by the index formula 2 is determined assuming that the symptom prevalence of the gastric cancer group is 0.038, the cutoff value is 0.125, and a sensitivity of 98%, a specificity of 99%, a positive predictive value of 92%, a negative predictive value of 99%, and a correct diagnostic rate of 99% are obtained. Thus, the index formula 2 is found to be a useful index with high diagnostic performance. In addition to that, a plurality of logistic regression equations having a discrimination performance equivalent to that of the index formula 2 is obtained. Those logistic regression equations are presented in FIG. 31, FIG. 32, FIG. 33, and FIG. 34. The respective values of the coefficients for the equations presented in FIG. 31, FIG. 32, FIG. 33, and FIG. 34, and 95% confidence intervals thereof may be values multiplied by a real number, and the values of the constant terms and 95% confidence intervals thereof may be values obtained by addition, subtraction, multiplication or division by an arbitrary real constant.

### Example 4

The sample data used in Example 1 is used. Indices by which the performance of discriminating between the 2 groups of the gastric cancer group and the gastric cancer-free group is maximized with regard to gastric cancer are searched by linear discriminant analysis (explanatory variable coverage method), and a linear discriminant composed of Asn, Orn, Phe, His, Gln, and Tyr (the numerical coefficients of the amino acid explanatory variables Asn, Orn, Phe, His, Gln, and Tyr are, in the same order, 33.35±1.69, 9.85±1.67, 12.62±2.70, −15.80±2.48, −1.00±0.35, and −9.02±2.16, respectively) is obtained as an index formula 3.

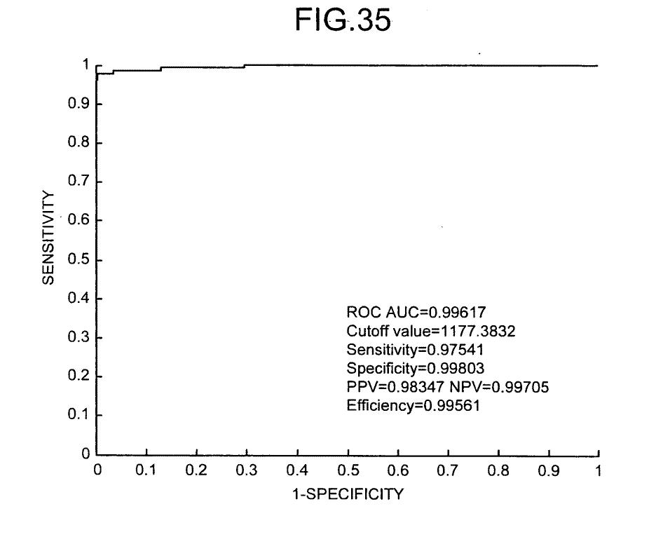

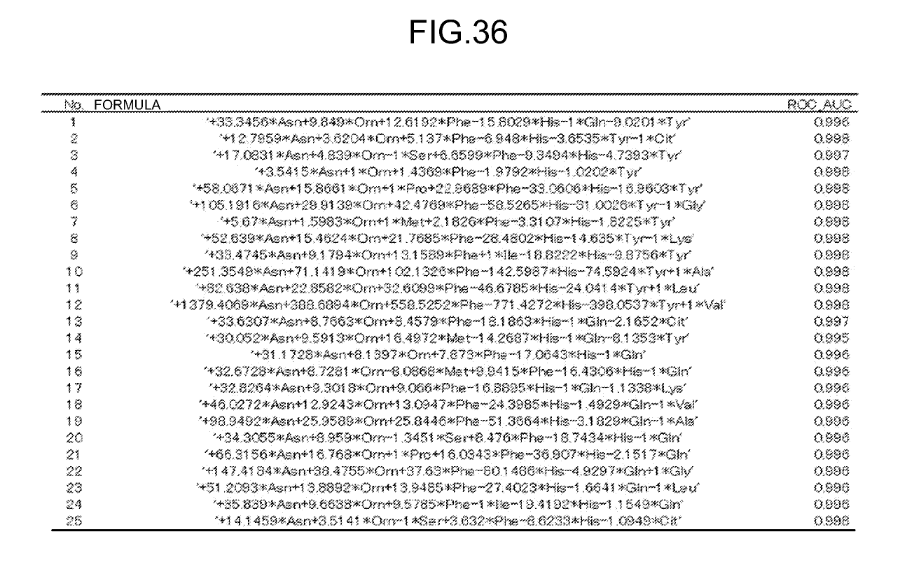

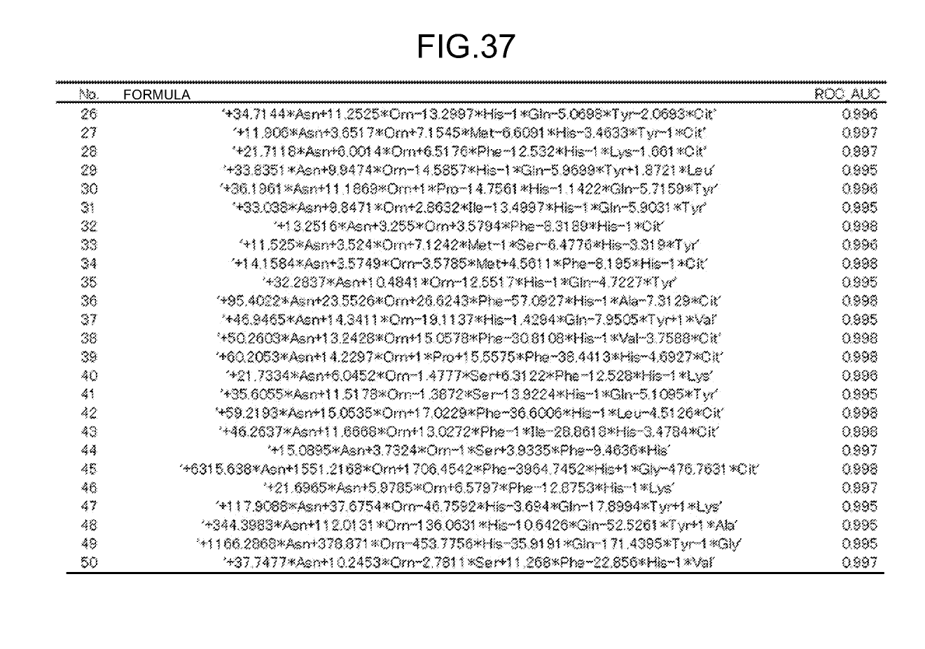

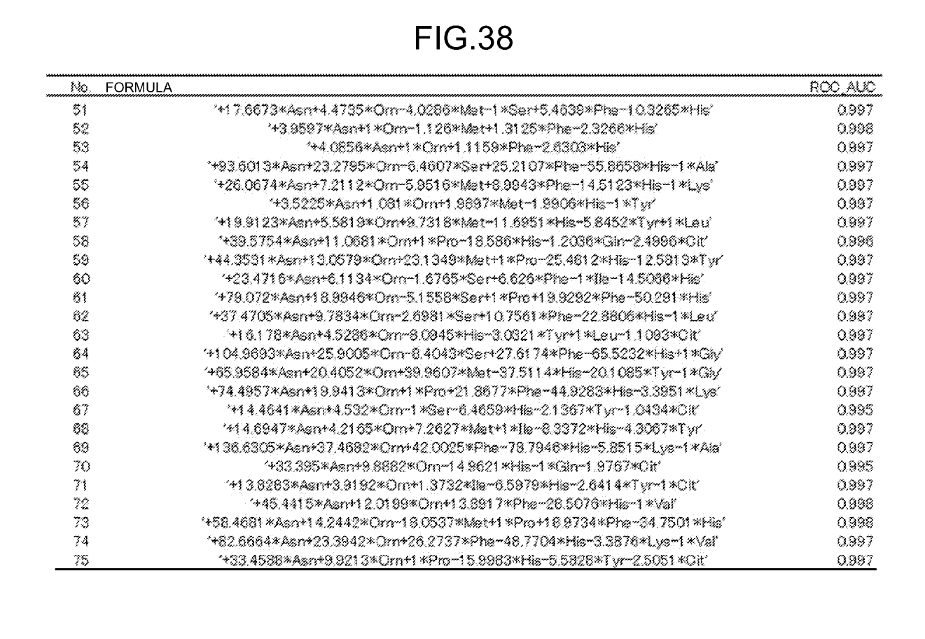

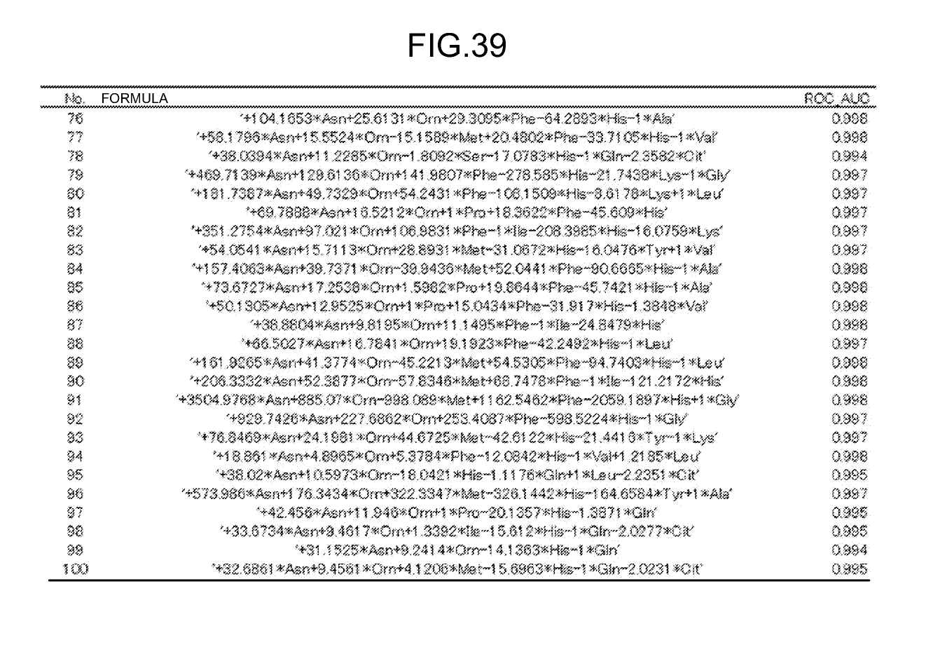

The performance for diagnosis of gastric cancer based on the index formula 3 is evaluated based on the AUC of the ROC curve (FIG. 35) in connection with the discrimination between the 2 groups of the gastric cancer group and the gastric cancer-free group, and an AUC of 0.996±0.003 (95% confidence interval: 0.991 to 1.00) is obtained. Thus, the index formula 3 is found to be a useful index with high diagnostic performance. When the optimum cutoff value for the discrimination between the 2 groups of the gastric cancer group and the gastric cancer-free group by the index formula 3 is determined assuming that the symptom prevalence of the gastric cancer group is 0.038, the cutoff value is 1177, and a sensitivity of 98%, a specificity of 99%, a positive predictive value of 98%, a negative predictive value of 99%, and a correct diagnostic rate of 99% are obtained. Thus, the index formula 3 is found to be a useful index with high diagnostic performance. In addition to that, a plurality of linear discriminants having a discrimination performance equivalent to that of the index formula 3 is obtained. Those linear discriminants are presented in FIG. 36, FIG. 37, FIG. 38, and FIG. 39. The respective values of the coefficients for the discriminants presented in FIG. 36, FIG. 37, FIG. 38, and FIG. 39, and 95% confidence intervals thereof may be values multiplied by a real number, and the values of the constant terms and 95% confidence intervals thereof may be values obtained by addition, subtraction, multiplication or division by an arbitrary real constant.

### Example 5

The sample data used in Example 1 is used. Gastric cancer pathological stages (Ia, Ib, II, IIIa, IIIb, and IV) with respect to gastric cancer are subjected to canonical correlation analysis with data of wall invasion depth, the presence or absence of histologic peritoneal dissemination, the presence or absence of histologic liver metastasis, and the presence or absence of histologic lymph node metastasis to convert the gastric cancer pathological stages into numbers. Indices having the highest correlation with stages are searched by multiple regression analysis (explanatory variable coverage method based on the BIC minimum criterion) to the obtained numerical data of the pathological stages, and a linear discriminant composed of His, Glu, Gly, and Arg (the numerical coefficients of the amino acid explanatory variables His, Glu, Gly, and Arg are, in the same order, −11.68±4.14, −3.91±±3.25, 1.00±0.66, and 3.22±2.39, respectively) is obtained as an index formula 4.

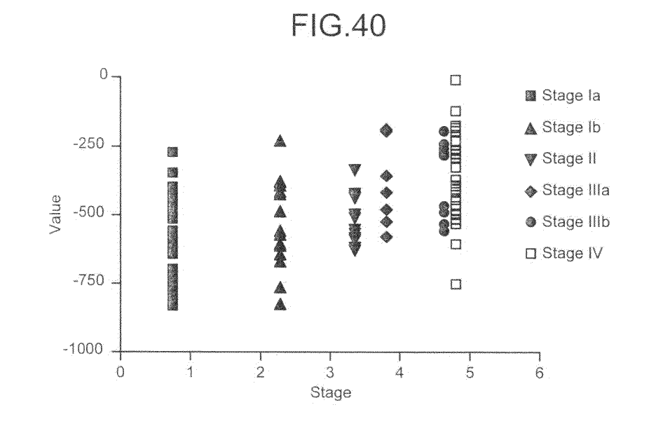

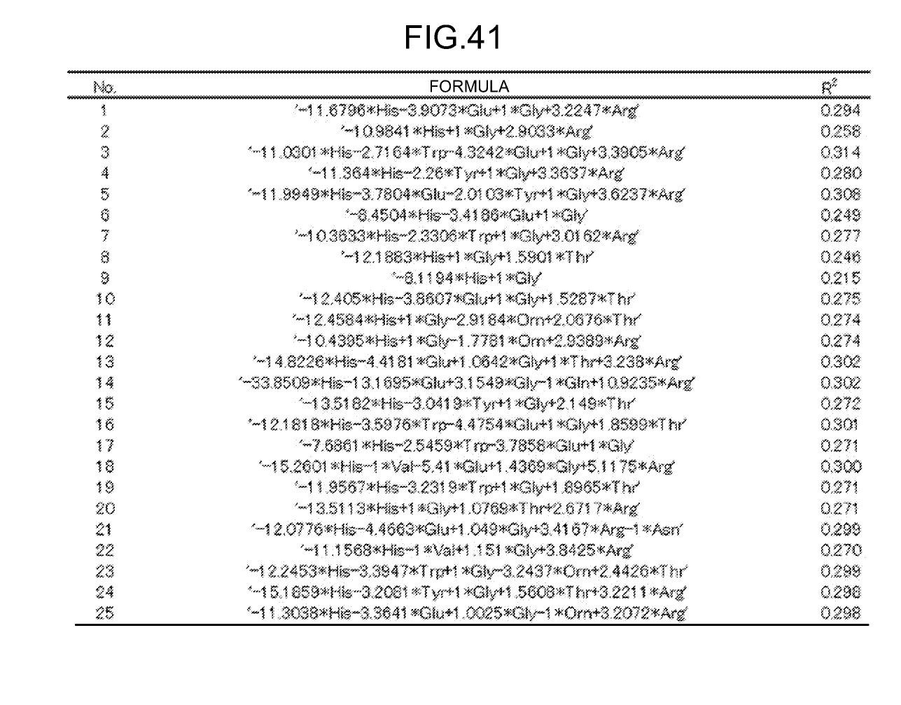

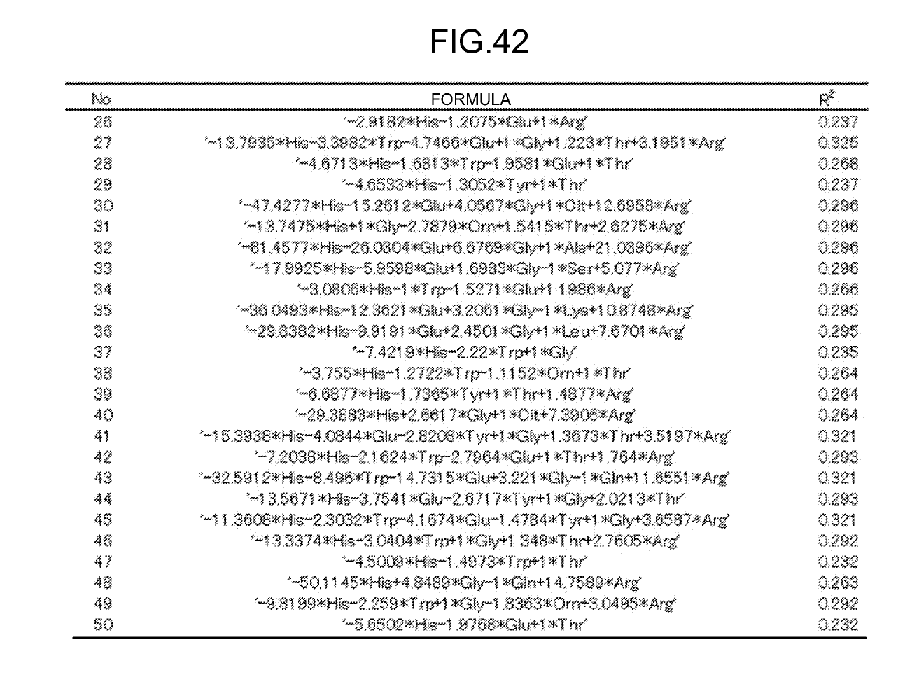

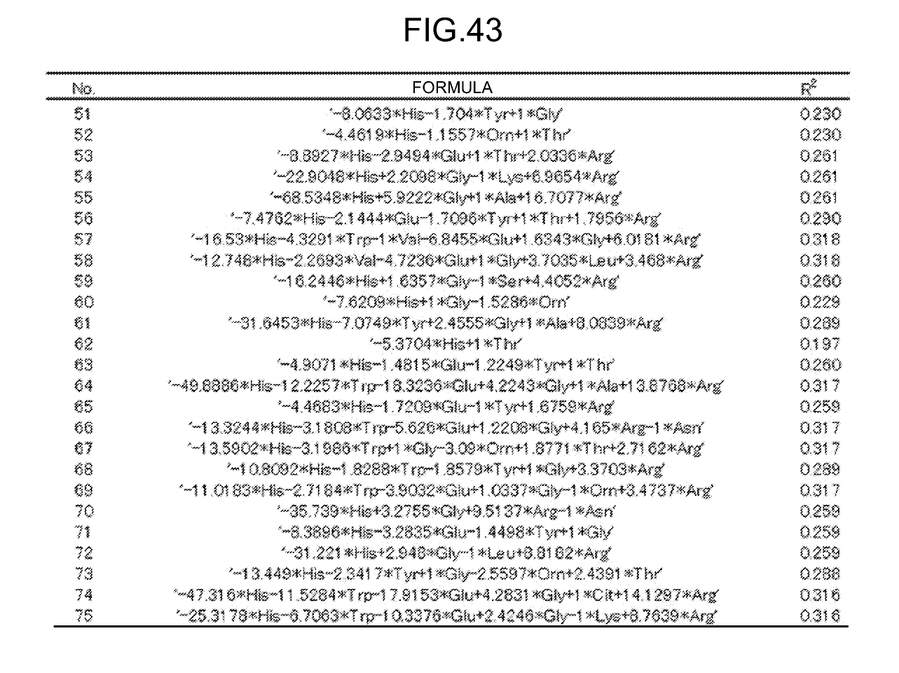

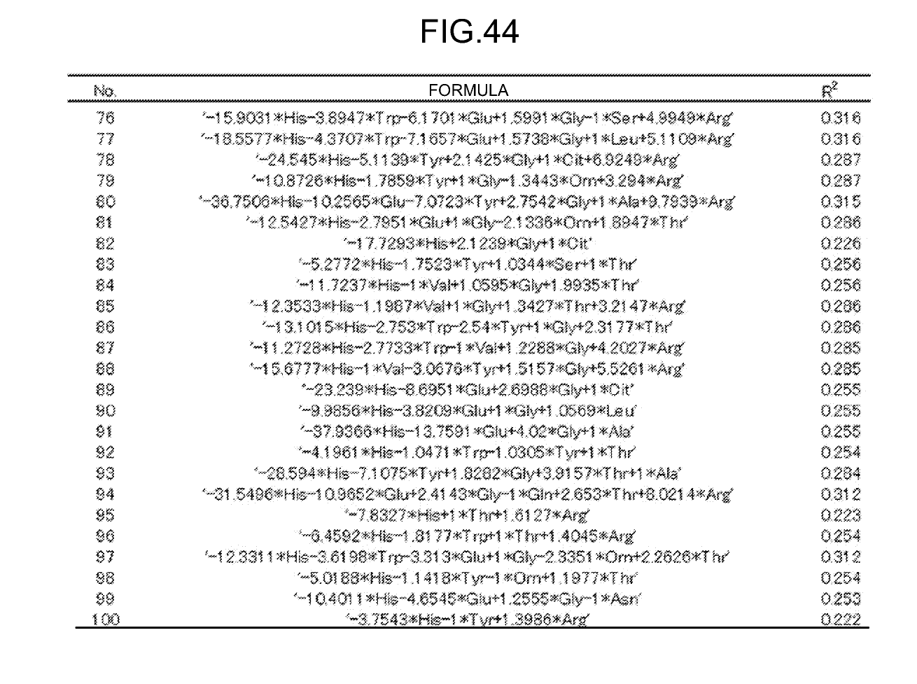

The Pearson correlation coefficient between the pathological stages which has been subjected to conversion into numbers and the value of the index formula 4 is 0.542 (95% confidence interval: 0.400 to 0.659, p<0.001). Thus, the index formula 4 is found to be a useful index with high diagnostic performance (FIG. 40). In addition to that, a plurality of linear discriminants having a discrimination performance equivalent to that of the index formula 4 is obtained. Those linear discriminants are presented in FIG. 41, FIG. 42, FIG. 43, and FIG. 44. The respective values of the coefficients for the discriminants presented in FIG. 41, FIG. 42, FIG. 43, and FIG. 44, and 95% confidence intervals thereof may be values multiplied by a real number, and the values of the constant terms and 95% confidence intervals thereof may be values obtained by addition, subtraction, multiplication or division by an arbitrary real constant.

### Example 6

The sample data used in Example 1 is used. Using a method described in International Publication WO 2004/052191 Pamphlet that is an international application filed by the present applicant, indices having the highest correlation with stages are eagerly searched to gastric cancer pathological stages (Ia, Ib, II, IIIa, IIIb, and IV) with respect to gastric cancer, and an index formula 5 is obtained among a plurality of indices having a equivalent performance.

(Gly)/(Glu+Trp+Val)+(Arg)/(His) Index formula 5

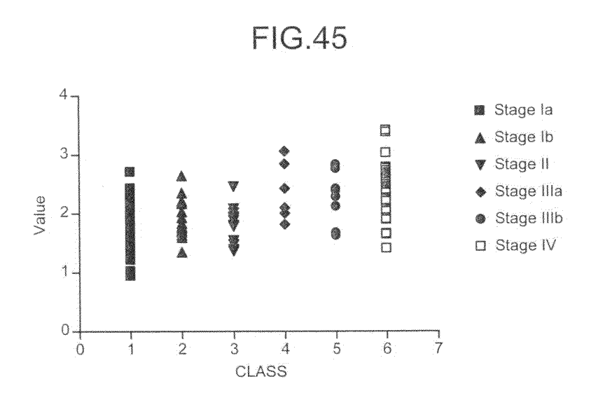

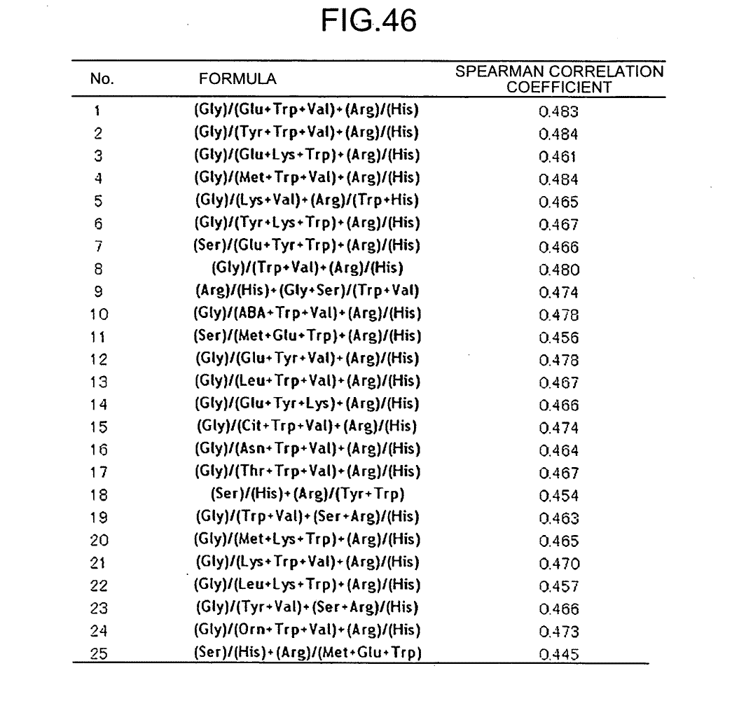

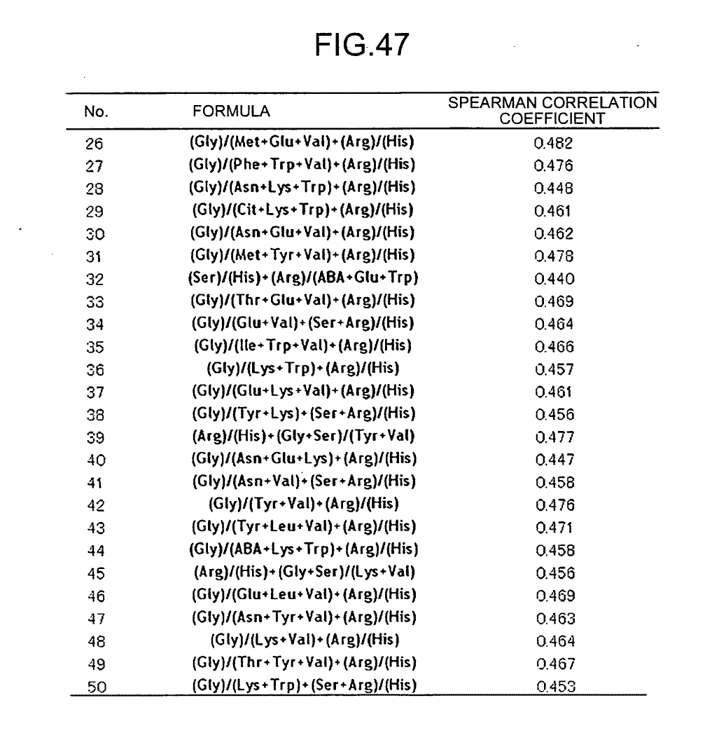

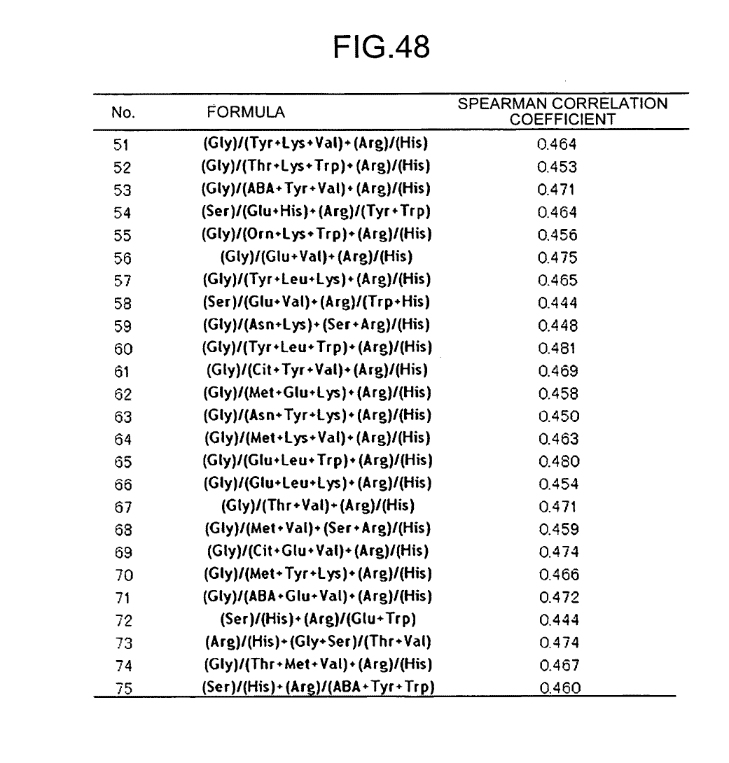

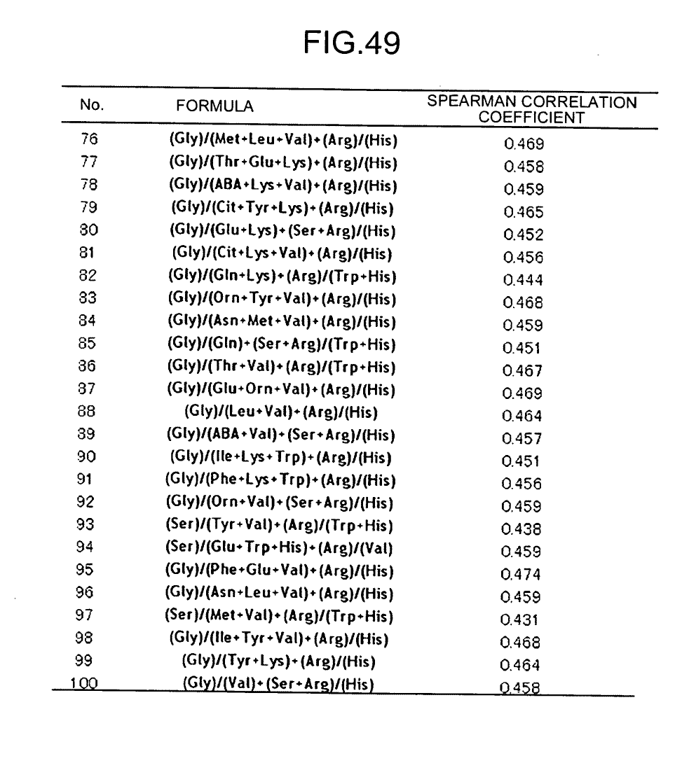

The Spearman rank correlation coefficient between the pathological stages and the value of the index formula 5 is 0.482 (95% confidence interval: 0.324 to 0.615, p<0.001). Thus, the index formula 5 is found to be a useful index with high diagnostic performance (FIG. 45). In addition to that, a plurality of index formulae having a discrimination performance equivalent to that of the index formula 5 is obtained. Those index formulae are presented in FIG. 46, FIG. 47, FIG. 48, and FIG. 49.

### Example 7

Using a method described in International Publication WO 2004/052191 Pamphlet that is an international application filed by the present applicant, indices by which the performance of 2-group discrimination with respect to the presence or absence of lymph node metastasis of gastric cancer is maximized with regard to gastric cancer are eagerly searched, and an index formula 6 is obtained among a plurality of indices having an equivalent performance.

(Ile)/(Glu)+(Gly+Asn+Arg)/(His) Index formula 6

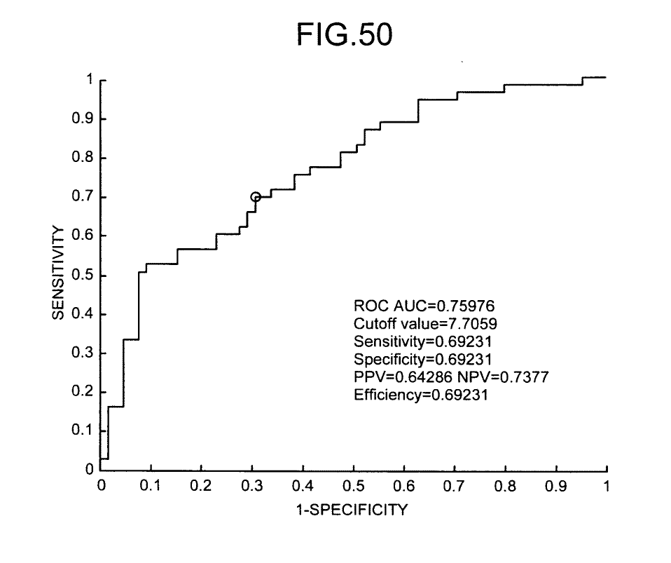

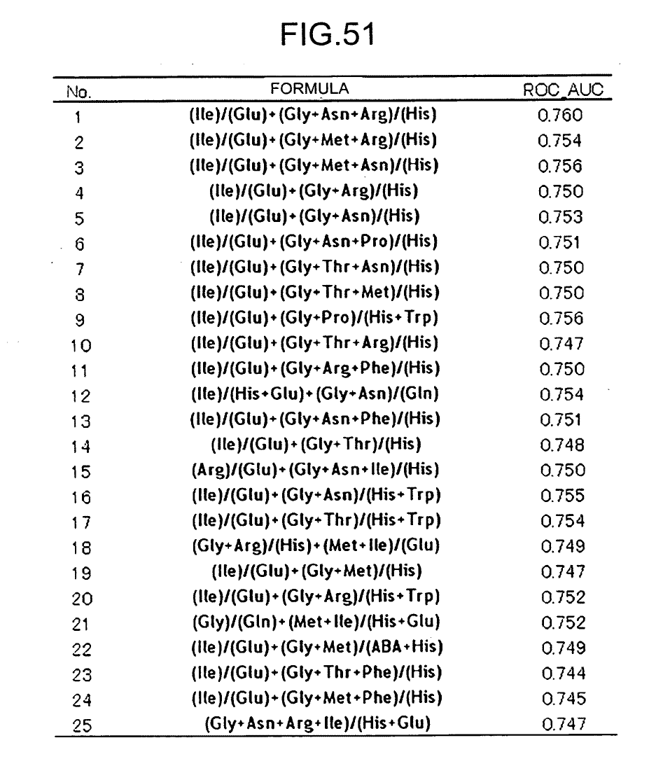

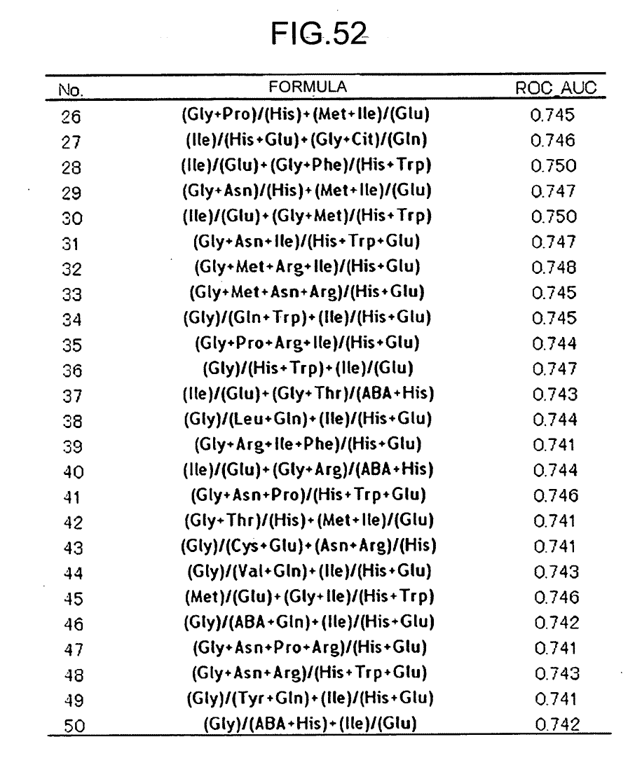

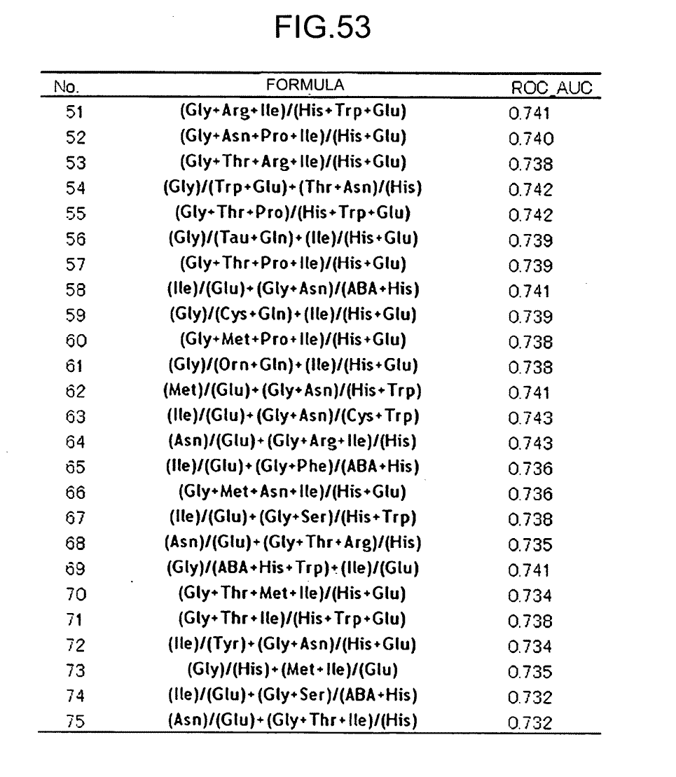

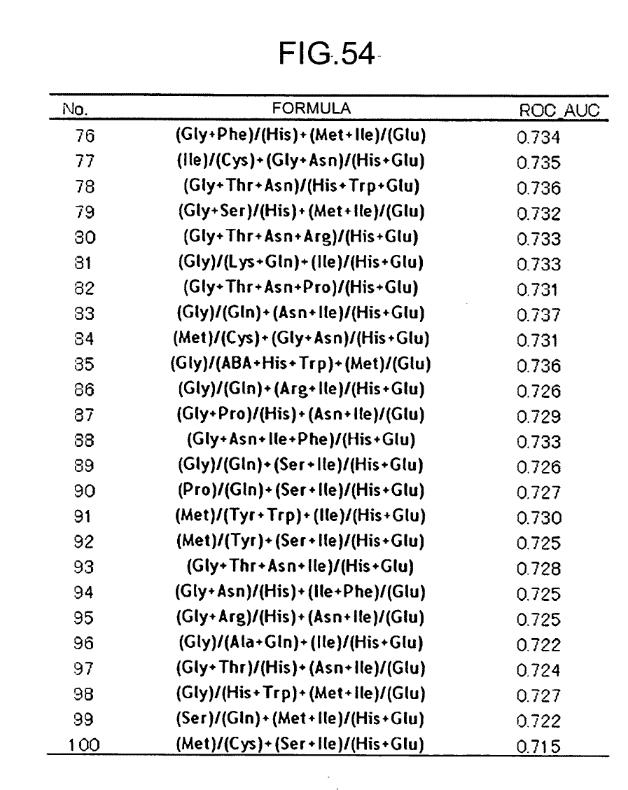

The performance for diagnosis of lymph node metastasis of gastric cancer based on the index formula 6 is evaluated based on the AUC of the ROC curve (FIG. 50) in connection with the discrimination between the 2 groups of a metastasis group and a metastasis-free group, and an AUC of 0.760±0.044 (95% confidence interval: 0.673 to 0.847) is obtained. When the optimum cutoff value for the discrimination between the 2 groups of the gastric cancer group and the gastric cancer-free group by the index formula 6 is determined assuming that the symptom prevalence of the gastric cancer group is 0.038, the cutoff value is 7.706, and a sensitivity of 69%, a specificity of 69%, a positive predictive value of 64%, a negative predictive value of 74%, and a correct diagnostic rate of 69% are obtained. Thus, the index formula 6 is found to be a useful index with high diagnostic performance. In addition to that, a plurality of fractional expressions having a discrimination performance equivalent to that of the index formula 6 is obtained. Those fractional expressions are presented in FIG. 51, FIG. 52, FIG. 53, and FIG. 54.

### Example 8

The sample data used in Example 1 is used. Indices by which the performance of 2-group discrimination of the presence or absence of lymph node metastasis of gastric cancer is maximized with regard to gastric cancer are searched by logistic analysis (explanatory variable coverage method based on the BIC minimum criterion), and a logistic regression equation composed of His, Met, and Tyr (the numerical coefficients of the amino acid explanatory variables His, Met, and Tyr, and the constant terms are, in the same order, —0.067±0.009, 0.161±0.002, −0.045±0.025, and 2.476±1.319, respectively) is obtained as an index formula 7.

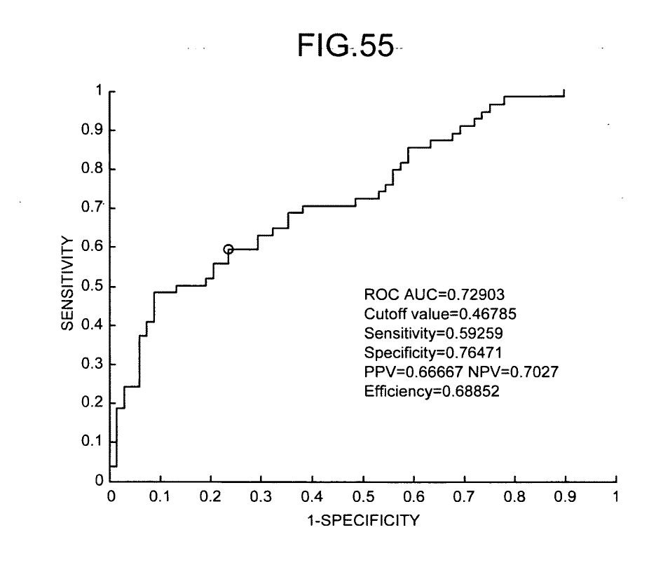

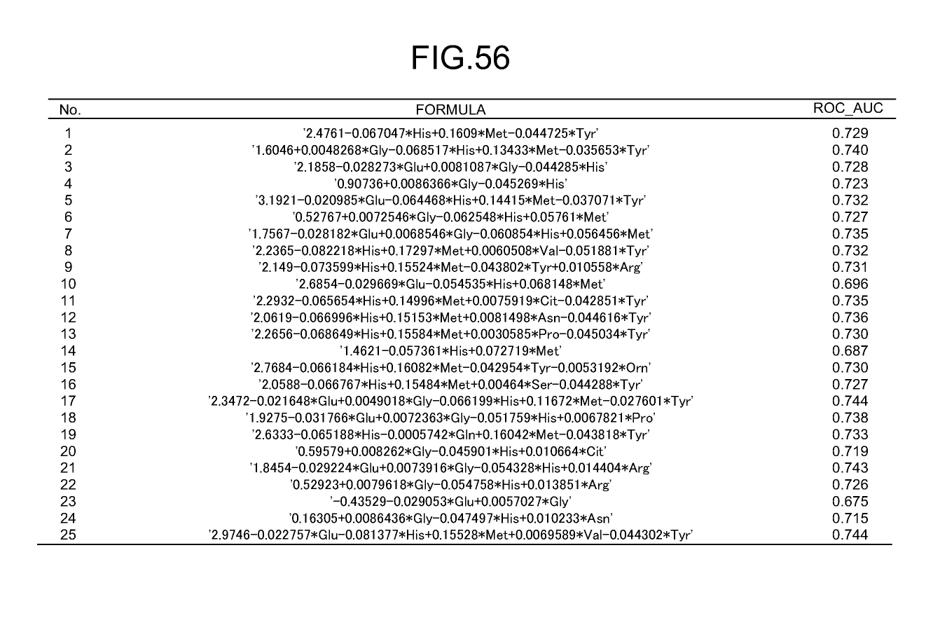

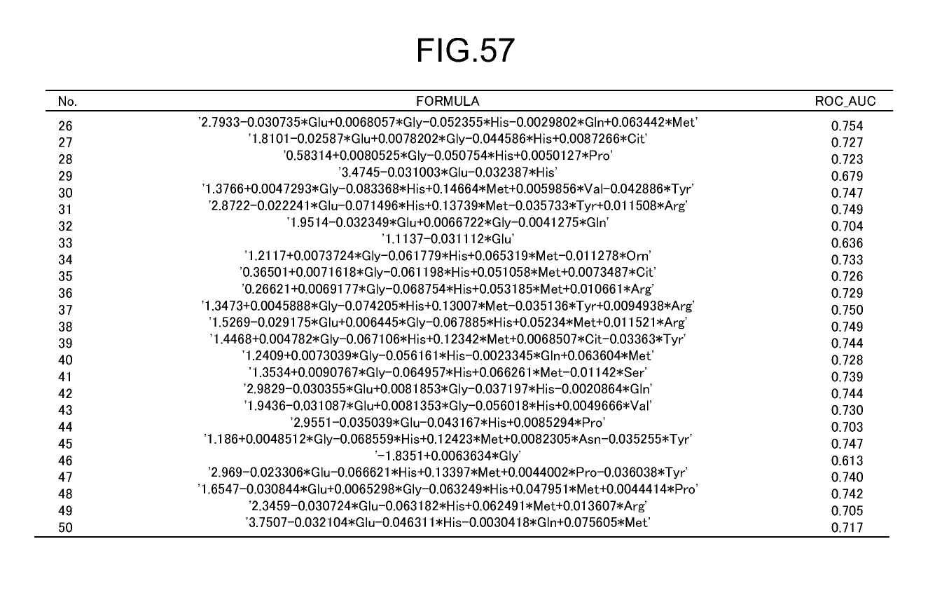

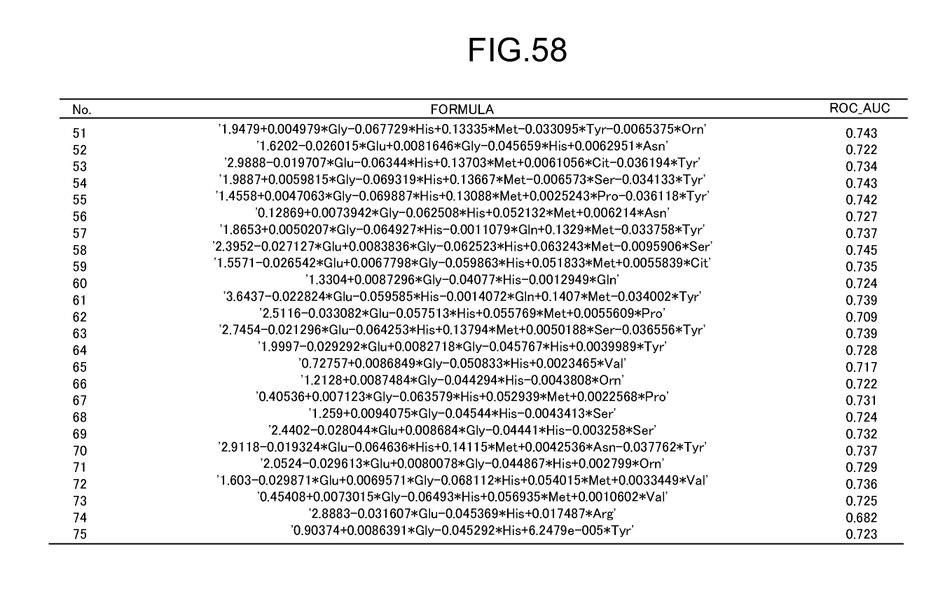

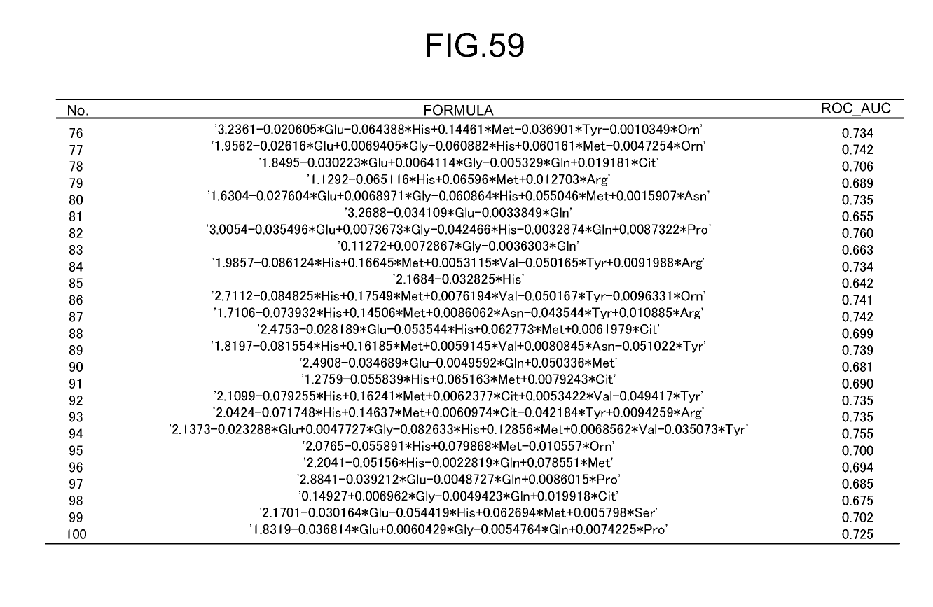

The performance for diagnosis of gastric cancer based on the index formula 7 is evaluated based on the AUC of the ROC curve (FIG. 55) in connection with the discrimination between the 2 groups of the metastasis group and the metastasis-free group, and an AUC of 0.729±0.046 (95% confidence interval: 0.631 to 0.819) is obtained. When the optimum cutoff value for the discrimination between the 2 groups of the metastasis group and the metastasis-free group by the index formula 7 is determined assuming that the symptom prevalence of the metastasis group was 0.443, the cutoff value is 0.468, and a sensitivity of 59%, a specificity of 76%, a positive predictive value of 67%, a negative predictive value of 70%, and a correct diagnostic rate of 69% are obtained. Thus, the index formula 7 is found to be a useful index with high diagnostic performance. In addition to that, a plurality of linear discriminants having a discrimination performance equivalent to that of the index formula 7 is obtained. Those logistic regression equations are presented in FIG. 56, FIG. 57, FIG. 58, and FIG. 59. The respective values of the coefficients for the equations presented in FIG. 56, FIG. 57, FIG. 58, and FIG. 59, and 95% confidence intervals thereof may be values multiplied by a real number, and the values of the constant terms and 95% confidence intervals thereof may be values obtained by addition, subtraction, multiplication or division by an arbitrary real constant.

### Example 9

The sample data used in Example 1 is used. Indices by which the performance of 2-group discrimination of the presence or absence of lymph node metastasis of gastric cancer is maximized with regard to gastric cancer are searched by linear discriminant analysis (explanatory variable coverage method), and a linear discriminant composed of His, Met, and Tyr (the numerical coefficients of the amino acid explanatory variables His, Met, and Tyr are, in the same order, −1.885±0.982, 3.680±1.821, and −1.000±0.704, respectively) is obtained as an index formula 8.

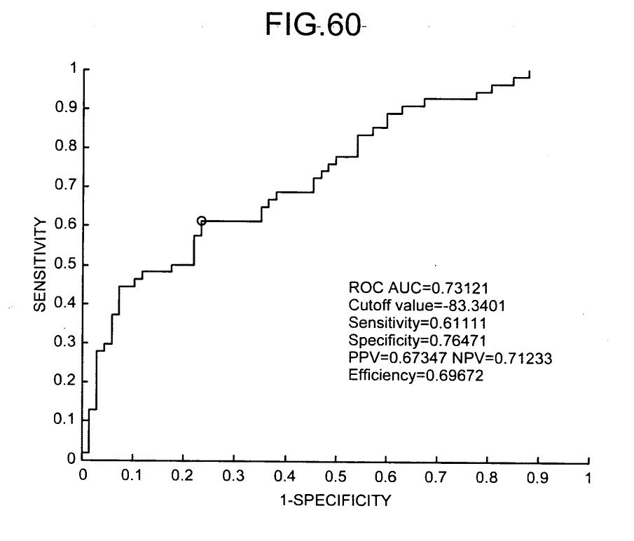

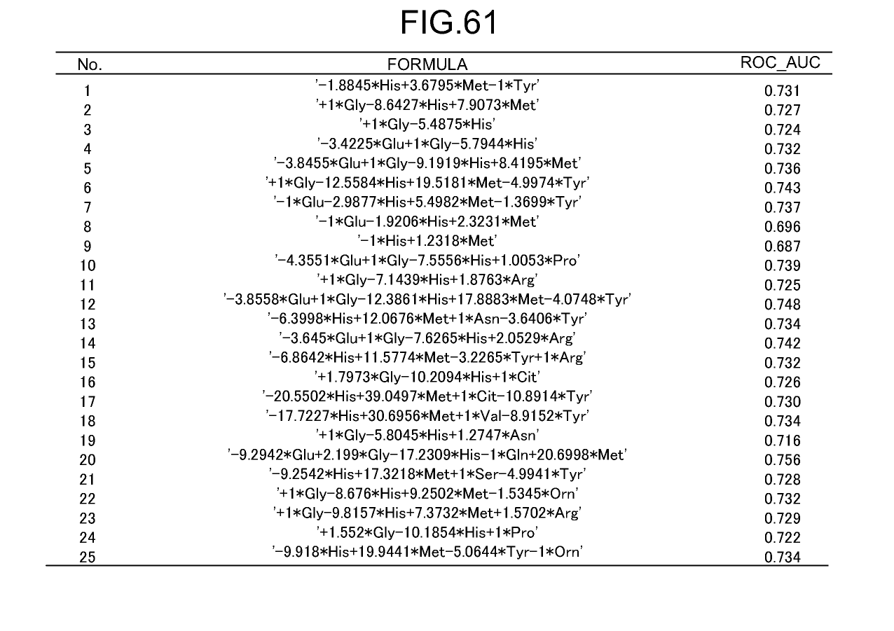

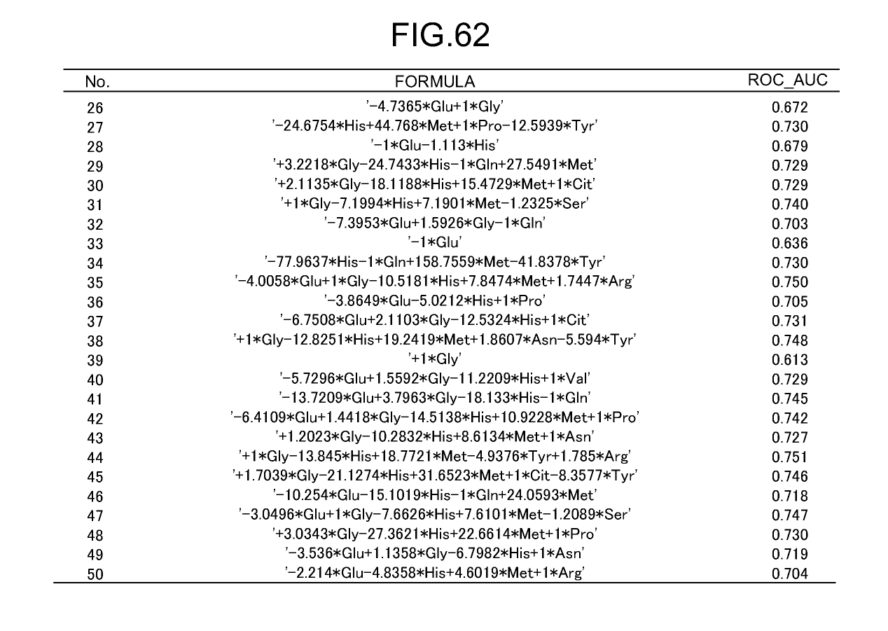

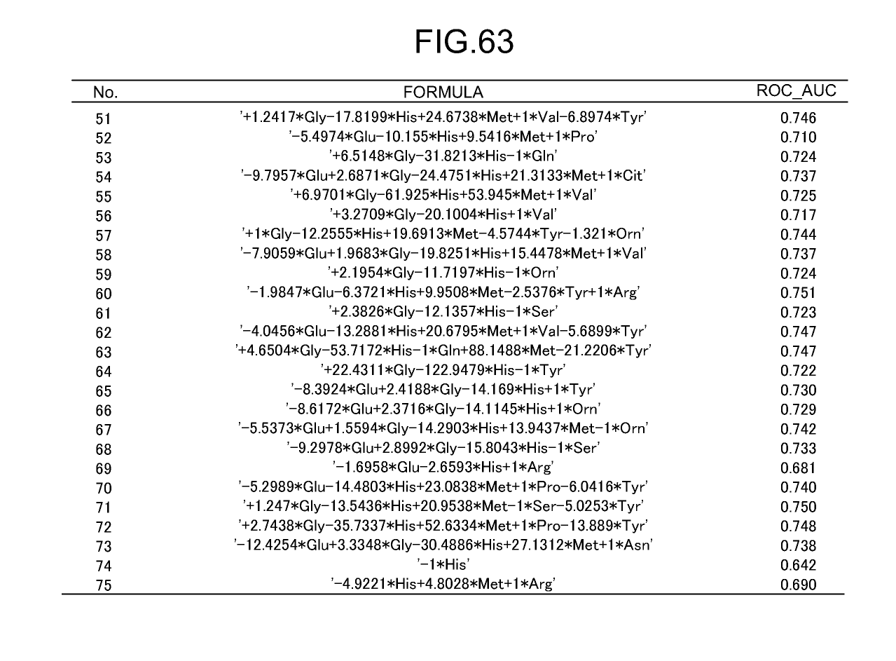

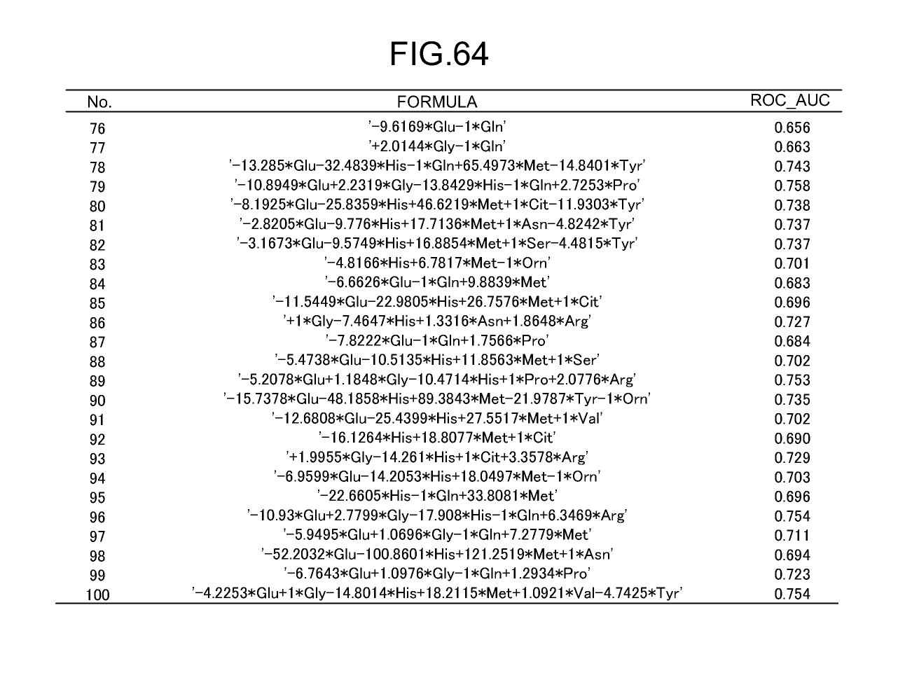

The performance for diagnosis of gastric cancer based on the index formula 8 is evaluated based on the AUC of the ROC curve (FIG. 60) in connection with the discrimination between the 2 groups of the metastasis group and the metastasis-free group, and an AUC of 0.731±0.046 (95% confidence interval: 0.642 to 0.821) is obtained. Thus, the index formula 8 is found to be a useful index with high diagnostic performance. When the optimum cutoff value for the discrimination between the 2 groups of the gastric cancer group and the gastric cancer-free group by the index formula 8 is determined assuming that the symptom prevalence of the metastasis group was 0.443, the cutoff value is −83.3, and a sensitivity of 61%, a specificity of 76%, a positive predictive value of 67%, a negative predictive value of 71%, and a correct diagnostic rate of 70% are obtained. Thus, the index formula 8 is found to be a useful index with high diagnostic performance. In addition to that, a plurality of linear discriminants having a discrimination performance equivalent to that of the index formula 8 is obtained. Those linear discriminants are presented in FIG. 61, FIG. 62, FIG. 63, and FIG. 64. The respective values of the coefficients for the discriminants presented in FIG. 61, FIG. 62, FIG. 63, and FIG. 64, and 95% confidence intervals thereof may be values multiplied by a real number, and the values of the constant terms and 95% confidence intervals thereof may be values obtained by addition, subtraction, multiplication or division by an arbitrary real constant.

### Example 10

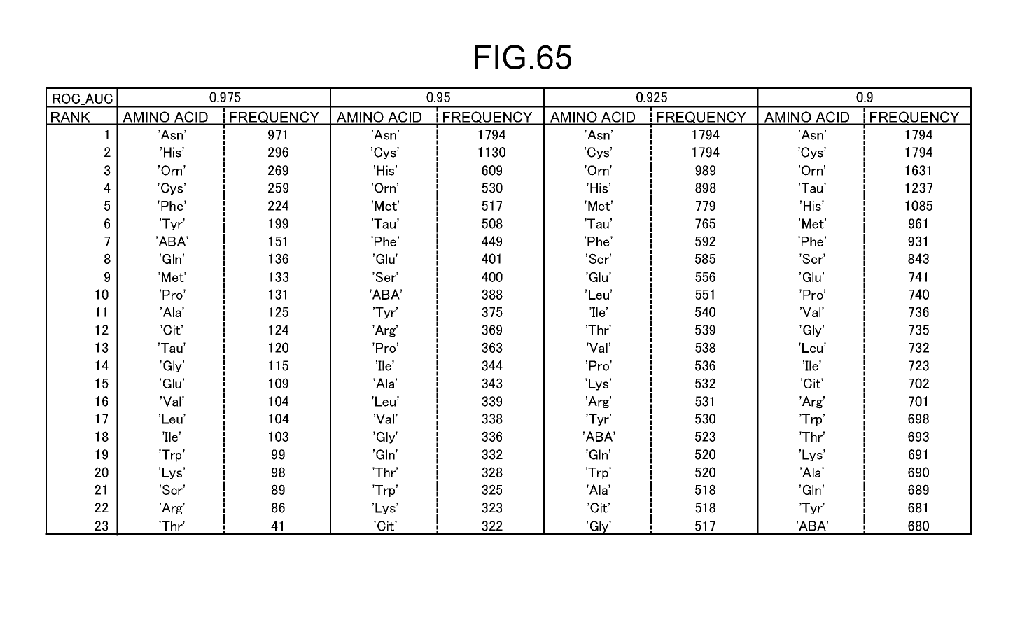

All linear discriminants for performing 2-group discrimination are extracted by the explanatory variable coverage method. Assuming that the maximum value of the amino acid explanatory variables appearing in each discriminant is 4, the area under the ROC curve of every discriminant satisfying this condition is calculated. Here, measurement is made of the frequency of each amino acid appearing in the discriminants in which the area under the ROC curve is equal to or greater than a certain threshold value, and as a result, Asn, Cys, His, Met, Orn, and Phe are verified to be included in top 10 amino acids which are always extracted at high frequency when areas under the ROC curve of 0.9, 0.925, 0.95, and 0.975 are respectively taken as the threshold values. Thus, it is made clear that multivariate discriminants using these amino acids as explanatory variables have an ability to discriminate between the 2 groups of the gastric cancer group and the gastric cancer-free group (FIG. 65).

### Example 11

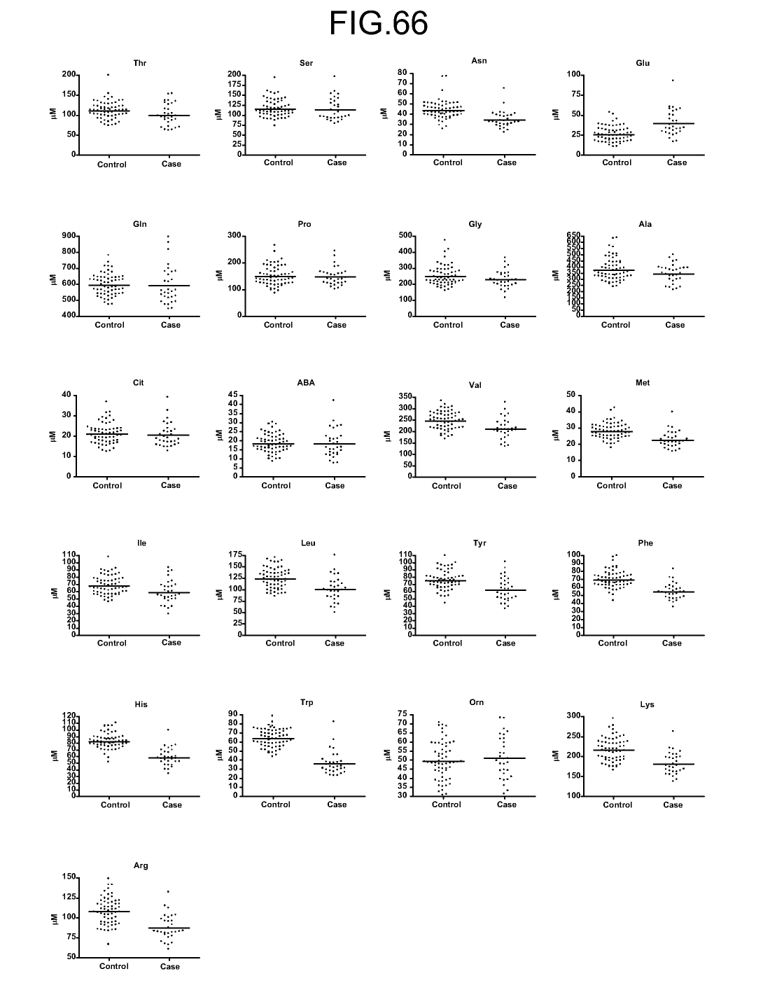

Blood samples of a group of gastric cancer patients diagnosed as gastric cancer by gastric biopsy, and blood samples of a group of gastric cancer-free subjects, are subjected to measurement of amino acid concentration in blood by the amino acid analysis method. FIG. 66 is a diagram showing the distribution of amino acid explanatory variables in the gastric cancer patients and the gastric cancer-free subjects. For the purpose of discrimination between the gastric cancer group and the gastric cancer-free group, a t-test between the 2 groups is performed.

In the gastric cancer group as compared with the gastric cancer-free group, Glu significantly increases, and Asn, Val, Met, Ile, Leu, Tyr, Phe, His, Trp, Lys, and Arg significantly decrease. Thus, it is made clear that the amino acid explanatory variables Glu, Asn, Val, Met, Ile, Leu, Tyr, Phe, His, Trp, Lys, and Arg have an ability to discriminate between the 2 groups of the gastric cancer group and the gastric cancer-free group.

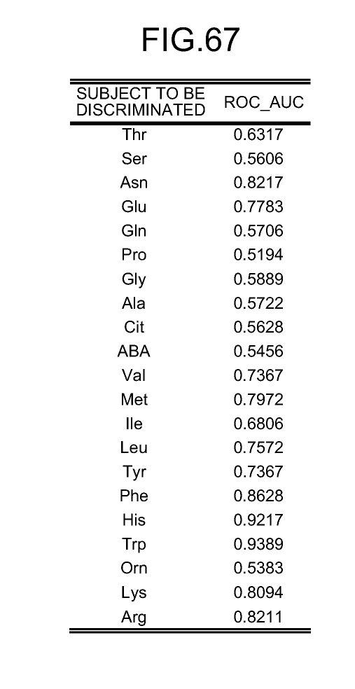

Furthermore, an evaluation using the AUC of the ROC curve is carried out for the discrimination between the 2 groups of the gastric cancer group and the gastric cancer-free group, and the AUC shows values larger than 0.75 for amino acid explanatory variables Asn, Glu, Met, Leu, Phe, His, Trp, Lys, and Arg (FIG. 67). Therefore, it is made clear that the amino acid explanatory variables Asn, Glu, Met, Leu, Phe, His, Trp, Lys, and Arg have an ability to discriminate between the 2 groups of the gastric cancer group and the gastric cancer-free group.

### Example 12

The sample data used in Example 11 is used. Using a method described in International Publication WO 2004/052191 Pamphlet that is an international application filed by the present applicant, indices by which the performance of discriminating between the 2 groups of the gastric cancer group and the gastric cancer-free group is maximized with regard to the discrimination of gastric cancer are eagerly searched, and an index formula 9 is obtained among a plurality of indices having an equivalent performance.

Glu/His+0.15×Ser/Trp-0.38×Arg/Pro Index formula 9

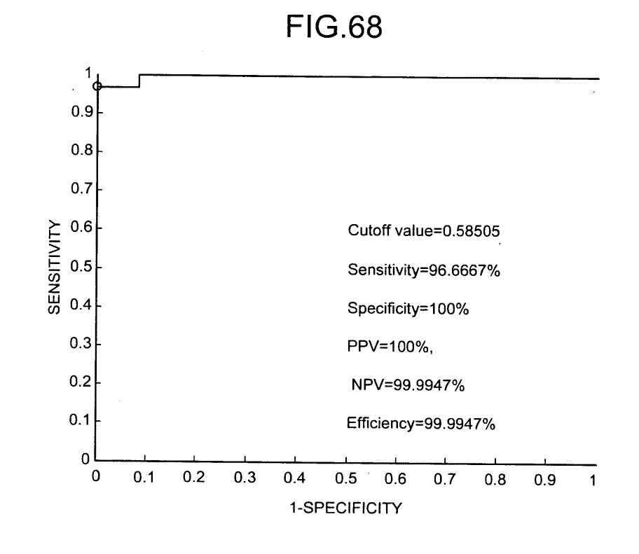

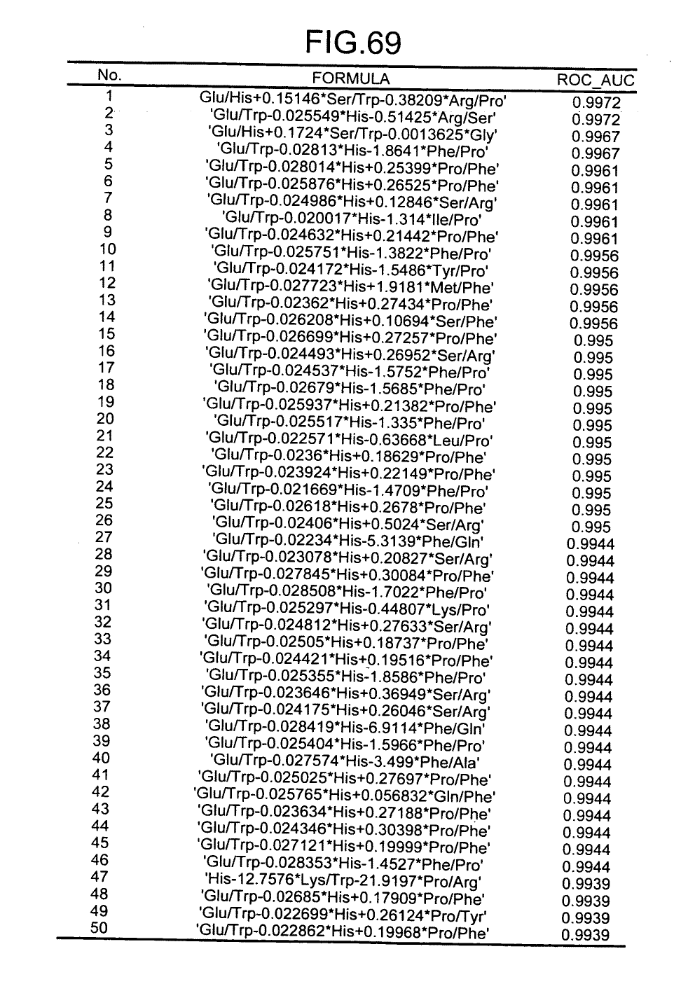

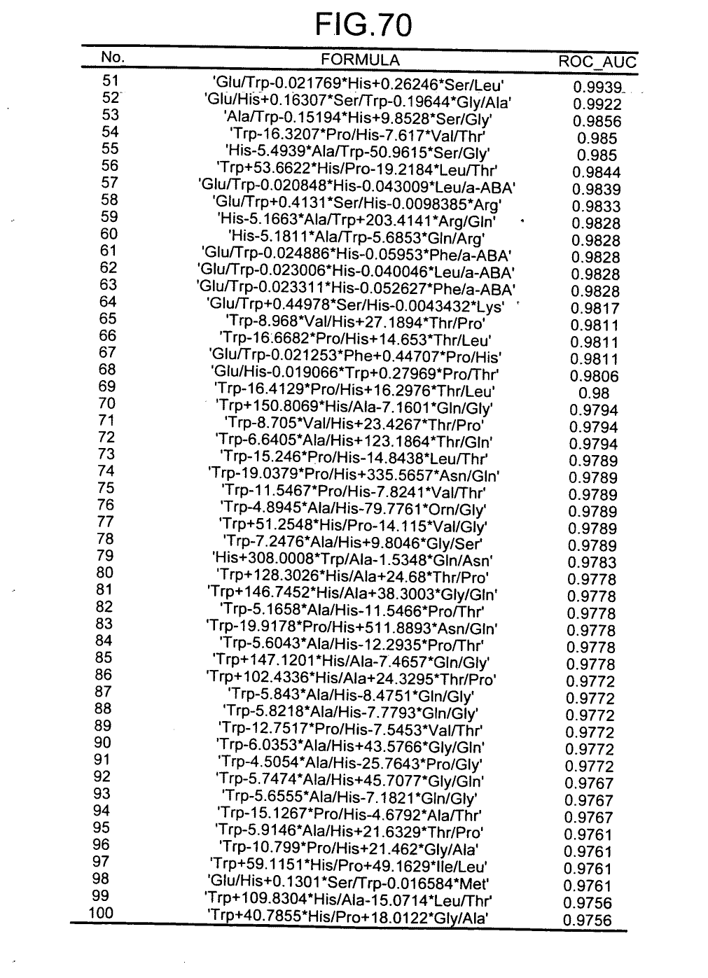

The performance for diagnosis of gastric cancer based on the index formula 9 is evaluated based on the AUC of the ROC curve (FIG. 68) in connection with the discrimination between the 2 groups of the gastric cancer group and the gastric cancer-free group, and an AUC of 0.997±0.003 (95% confidence interval: 0.991 to 1) is obtained. When the optimum cutoff value for the discrimination between the 2 groups of the gastric cancer group and the gastric cancer-free group by the index formula 9 is determined assuming that the symptom prevalence of the gastric cancer group is 0.16%, the cutoff value is 0.585, and a sensitivity of 96.67%, a specificity of 100.0%, a positive predictive value of 100.0%, a negative predictive value of 99.99%, and a correct diagnostic rate of 99.99% are obtained (FIG. 68). Thus, the index formula 9 is found to be a useful index with high diagnostic performance. In addition to that, a plurality of multivariate discriminants having a discrimination performance equivalent to that of the index formula 9 is obtained. Those multivariate discriminants are presented in FIG. 69 and FIG. 70. The respective values of the coefficients for the discriminants presented in FIG. 69 and FIG. 70 may be values multiplied by a real number, or values obtained by adding an arbitrary constant term.

### Example 13

The sample data used in Example 11 is used. Indices by which the performance of discriminating between the 2 groups of the gastric cancer group and the gastric cancer-free group is maximized with regard to gastric cancer are searched by logistic analysis (explanatory variable coverage method based on the BIC minimum criterion), and a logistic regression equation composed of Glu, Phe, His, and Trp (the numerical coefficients of the amino acid explanatory variables Glu, Phe, His, and Trp, and the constant terms are, in the same order, 0.1254±0.001, −0.0684±0.004, −0.1066±0.002, −0.1257±0.0027, and 12.9742±0.1855, respectively) is obtained as an index formula 10.

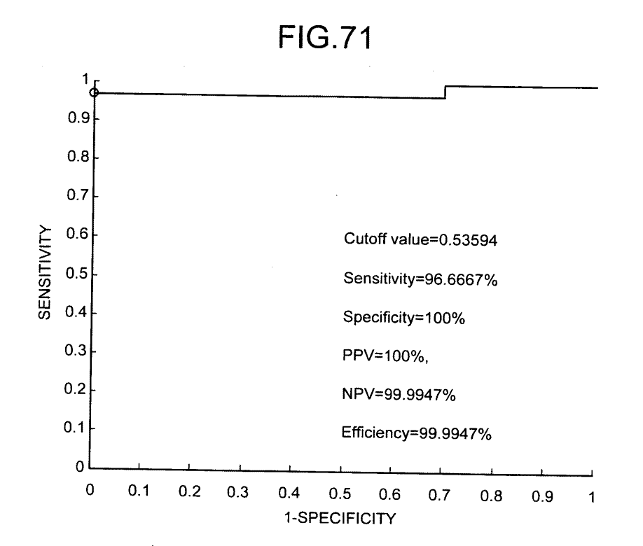

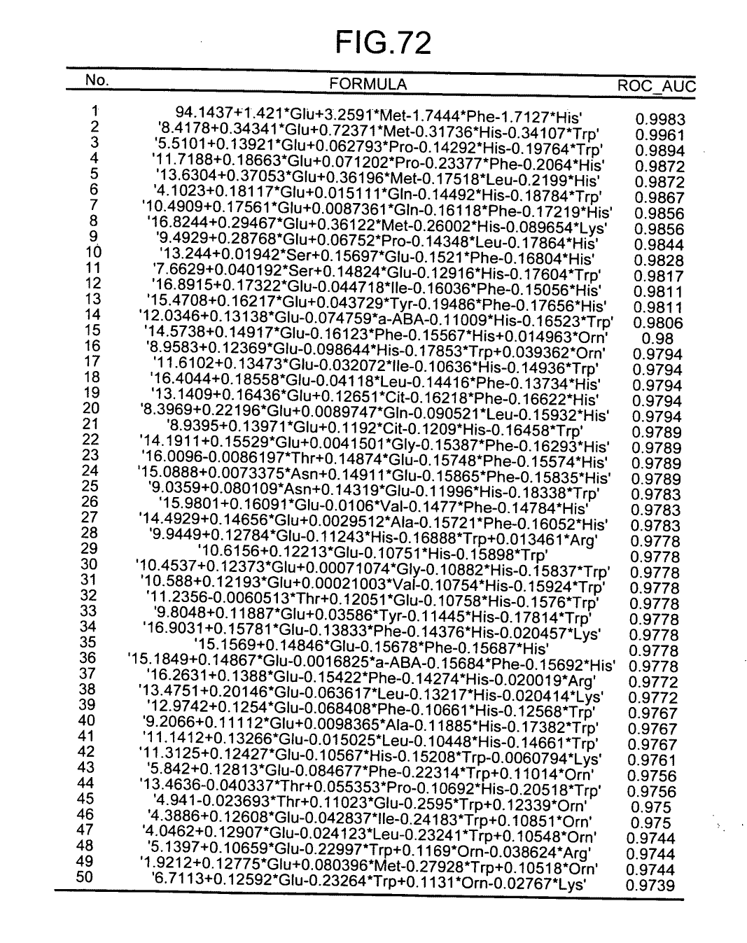

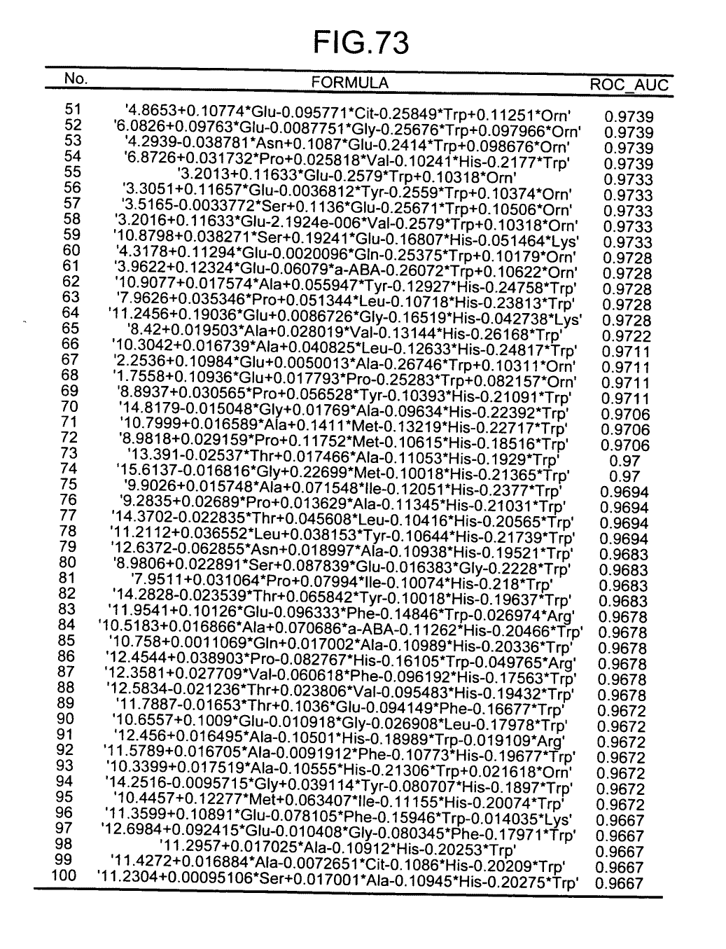

The performance for diagnosis of gastric cancer based on the index formula 10 is evaluated based on the AUC of the ROC curve (FIG. 71) in connection with the discrimination between the 2 groups of the gastric cancer group and the gastric cancer-free group, and an AUC of 0.977±0.023 (95% confidence interval: 0.932 to 1) is obtained. Thus, the index formula 10 is found to be a useful index with high diagnostic performance. When the optimum cutoff value for the discrimination between the 2 groups of the gastric cancer group and the gastric cancer-free group by the index formula 10 is determined assuming that the symptom prevalence of the gastric cancer group is 0.16%, the cutoff value is 0.536, and a sensitivity of 96.7%, a specificity of 100%, a positive predictive value of 100%, a negative predictive value of 99.99%, and a correct diagnostic rate of 99.99% are obtained (FIG. 71). Thus, the index formula 10 is found to be a useful index with high diagnostic performance. In addition to that, a plurality of logistic regression equations having a discrimination performance equivalent to that of the index formula 10 is obtained. Those logistic regression equations are presented in FIG. 72 and FIG. 73. The respective values of the coefficients for the equations presented in FIG. 72 and FIG. 73 may be values multiplied by a real number.

### Example 14

The sample data used in Example 11 is used. Indices by which the performance of discriminating between the 2 groups of the gastric cancer group and the gastric cancer-free group is maximized with regard to gastric cancer are searched by linear discriminant analysis (explanatory variable coverage method), and a linear discriminant function composed of Glu, Pro, His, and Trp (the numerical coefficients of the amino acid explanatory variables Glu, Pro, His, and Trp are, in the same order, 1±0.2, 0.2703±0.0085, −1.0845±0.0359, and −1.4648±0.0464, respectively) is obtained as an index formula 11.

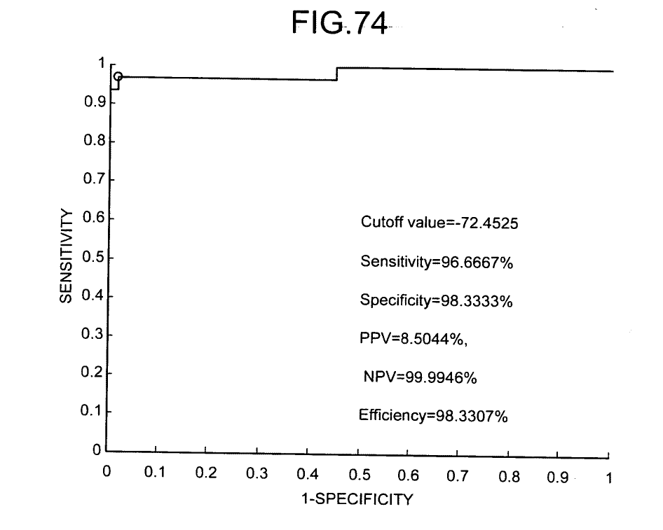

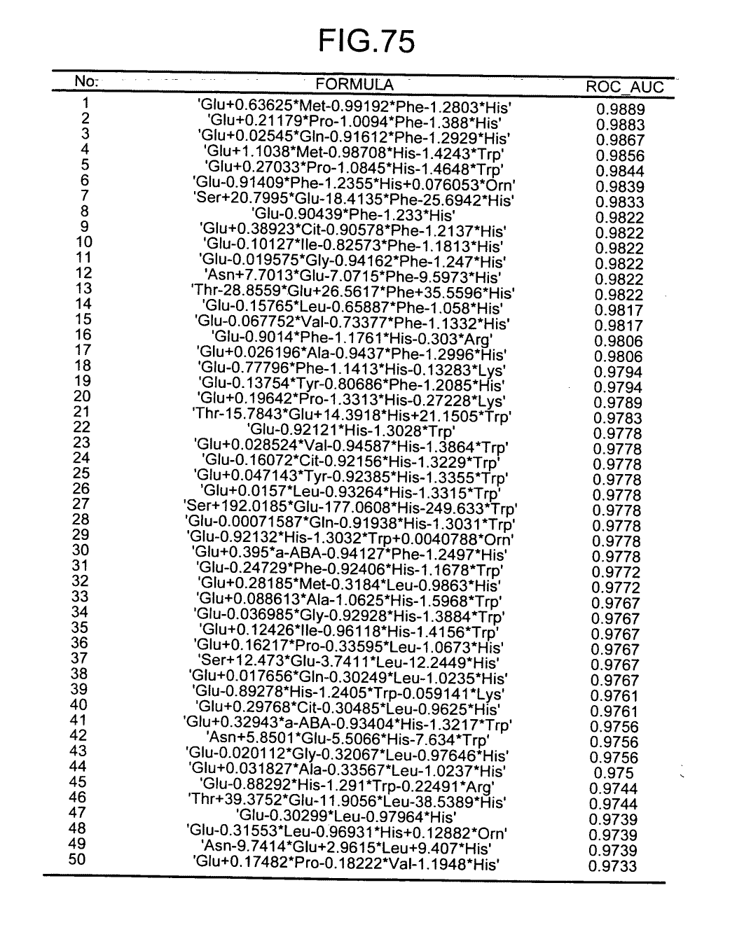

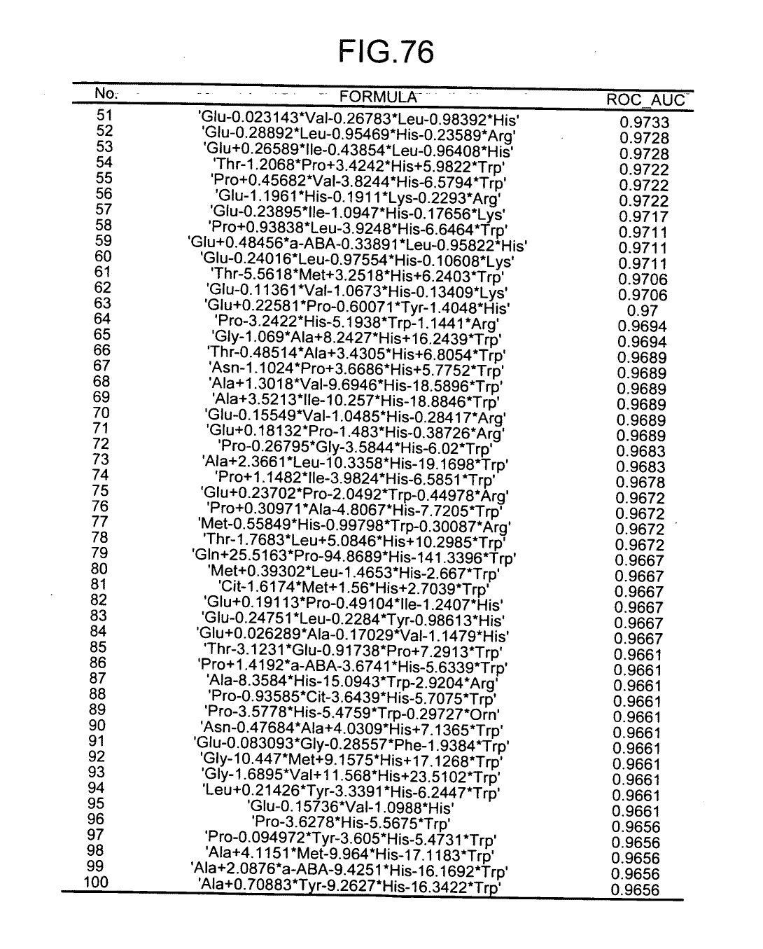

The performance for diagnosis of gastric cancer based on the index formula 11 is evaluated based on the AUC of the ROC curve (FIG. 74) in connection with the discrimination between the 2 groups of the gastric cancer group and the gastric cancer-free group, and an AUC of 0.984±0.015 (95% confidence interval: 0.955 to 1) is obtained. Thus, the index formula 11 is found to be a useful index with high diagnostic performance. When the optimum cutoff value for the discrimination between the 2 groups of the gastric cancer group and the gastric cancer-free group by the index formula 11 is determined assuming that the symptom prevalence of the gastric cancer group is 0.16%, the cutoff value is −72.45, and a sensitivity of 96.7%, a specificity of 98.3%, a positive predictive value of 8.50%, a negative predictive value of 99.99%, and a correct diagnostic rate of 98.33% are obtained (FIG. 74). Thus, the index formula 11 is found to be a useful index with high diagnostic performance. In addition to that, a plurality of linear discriminant functions having a discrimination performance equivalent to that of the index formula 11 is obtained. Those linear discriminant functions are presented in FIG. 75 and FIG. 76. The respective values of the coefficients for the functions presented in FIG. 75 and FIG. 76 may be values multiplied by a real number, or values obtained by adding an arbitrary constant term.

### Example 15

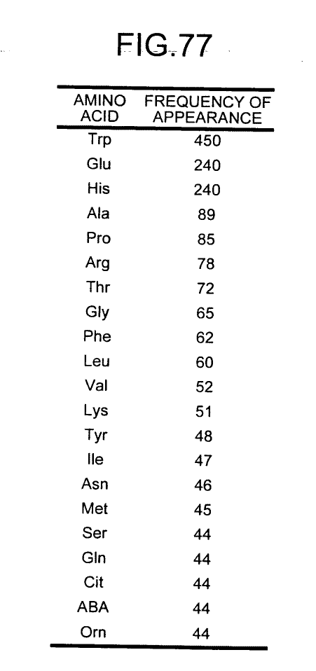

The sample data used in Example 11 is used. All linear discriminants for performing 2-group discrimination of the gastric cancer group and the gastric cancer-free group with regard to gastric cancer are extracted by the explanatory variable coverage method. Assuming that the maximum value of the amino acid explanatory variables appearing in each discriminant is 4, the area under the ROC curve of every discriminant satisfying this condition is calculated. Here, measurement is made of the frequency of each amino acid appearing in the discriminants in which the area under the ROC curve is in top 500, and as a result, Trp, Glu, His, Ala, and Pro are verified to be top 5 amino acids which are always extracted at high frequency. Thus, it is made clear that multivariate discriminants using these amino acids as explanatory variables have an ability to discriminate between the 2 groups of the gastric cancer group and the gastric cancer-free group (FIG. 77).

### Example 16

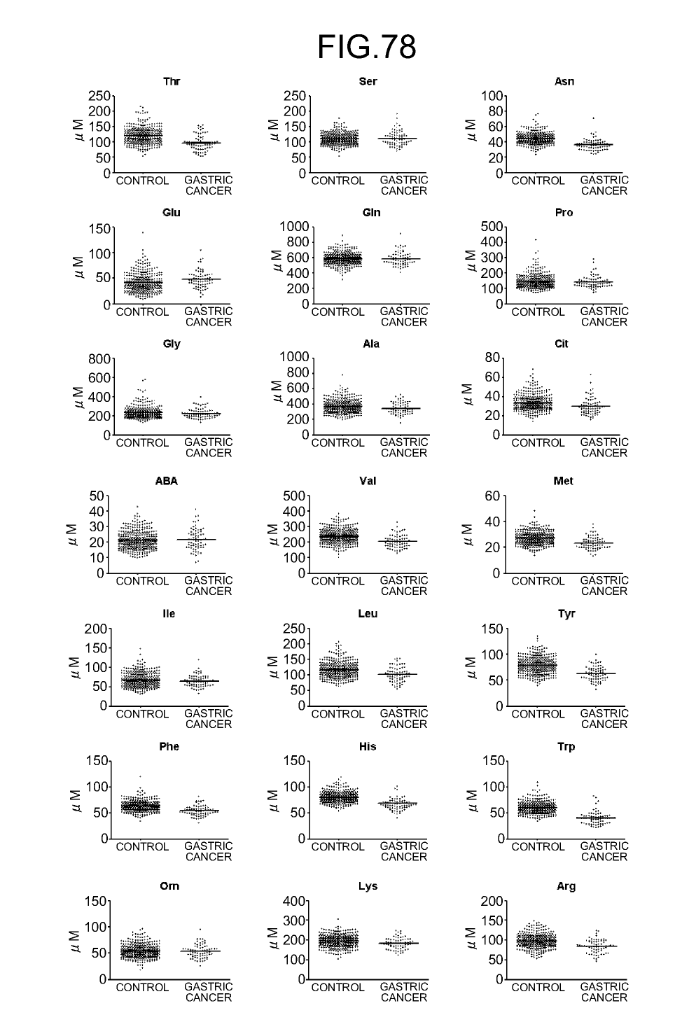

Blood samples of a group of gastric cancer patients diagnosed as gastric cancer by gastric biopsy, and blood samples of a group of gastric cancer-free subjects, are subjected to measurement of amino acid concentration in blood by the amino acid analysis method. FIG. 78 is a diagram showing the distribution of amino acid explanatory variables in the gastric cancer patients and the gastric cancer-free subjects. For the purpose of discrimination between the gastric cancer group and the gastric cancer-free group, Wilcoxon rank-sum test between the 2 groups is performed.

In the gastric cancer group as compared with the gastric cancer-free group, Glu significantly increases, and Thr, Asn, Ala, Cit, Val, Met, Leu, Tyr, Phe, His, Trp, Lys, and Arg significantly decrease. Thus, it is made clear that the amino acid explanatory variables Glu, Thr, Asn, Ala, Val, Met, Leu, Tyr, Phe, His, Trp, Lys, and Arg have an ability to discriminate between the 2 groups of the gastric cancer group and the gastric cancer-free group.

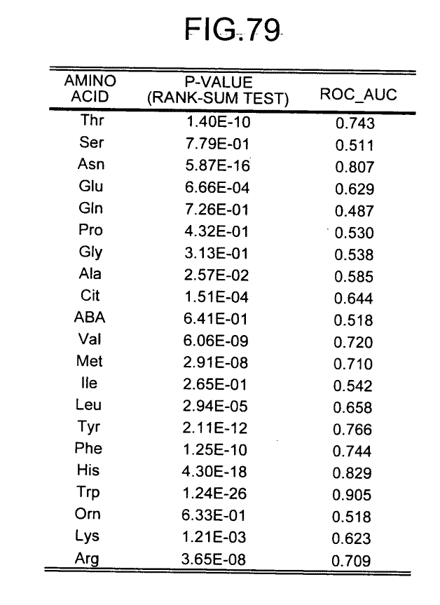

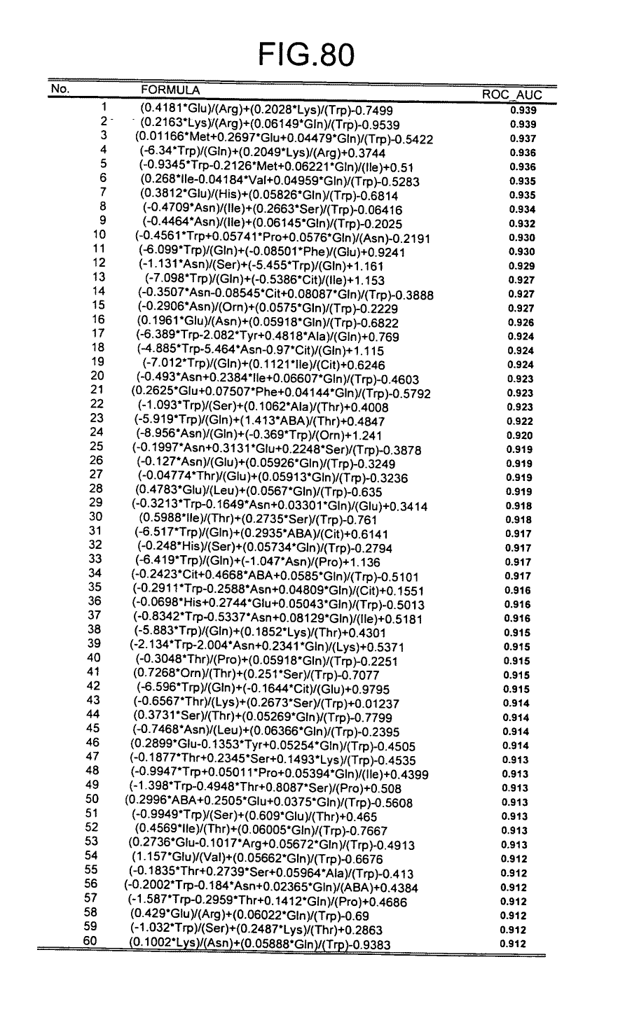

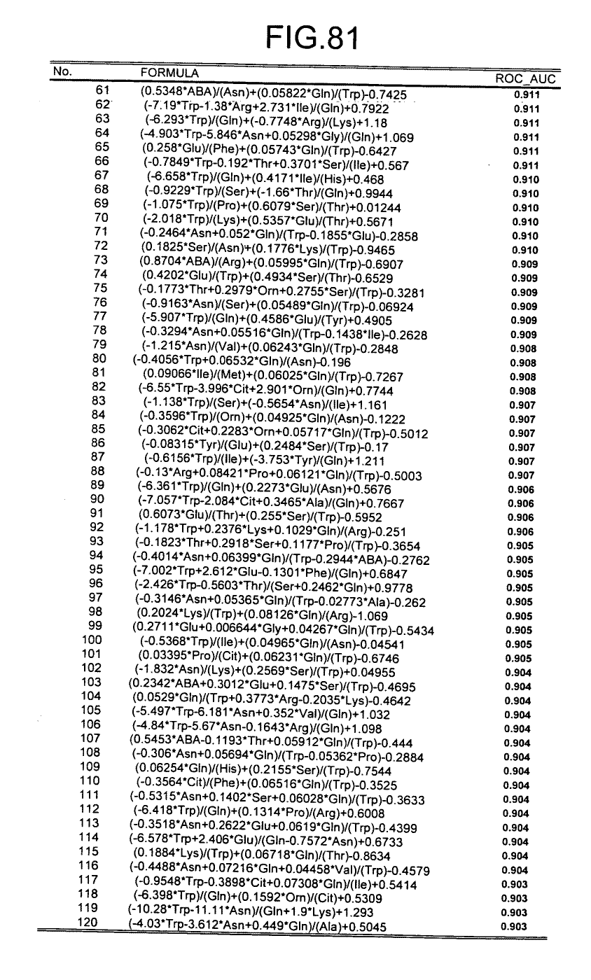

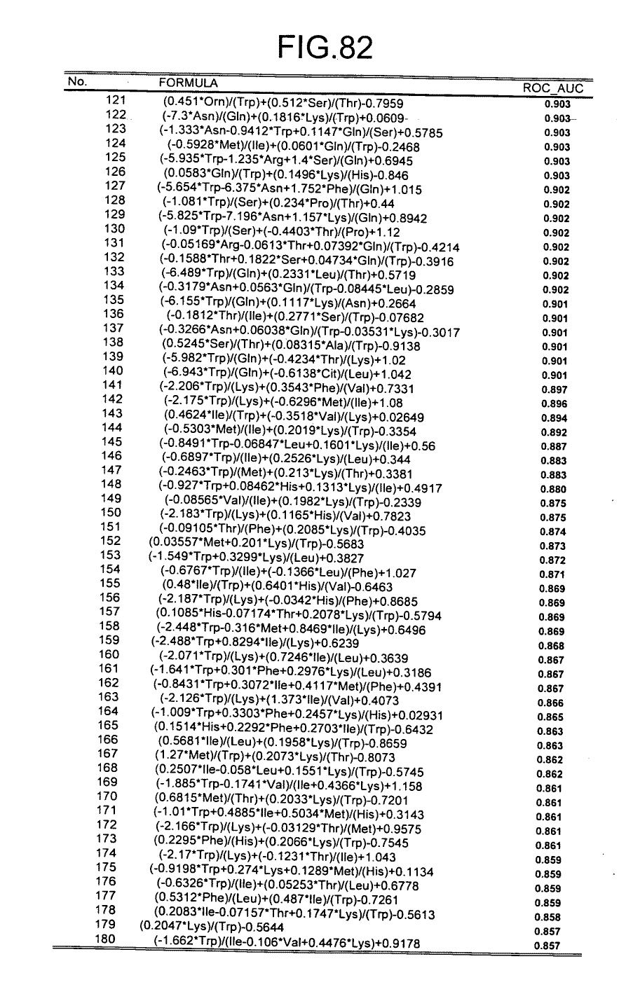

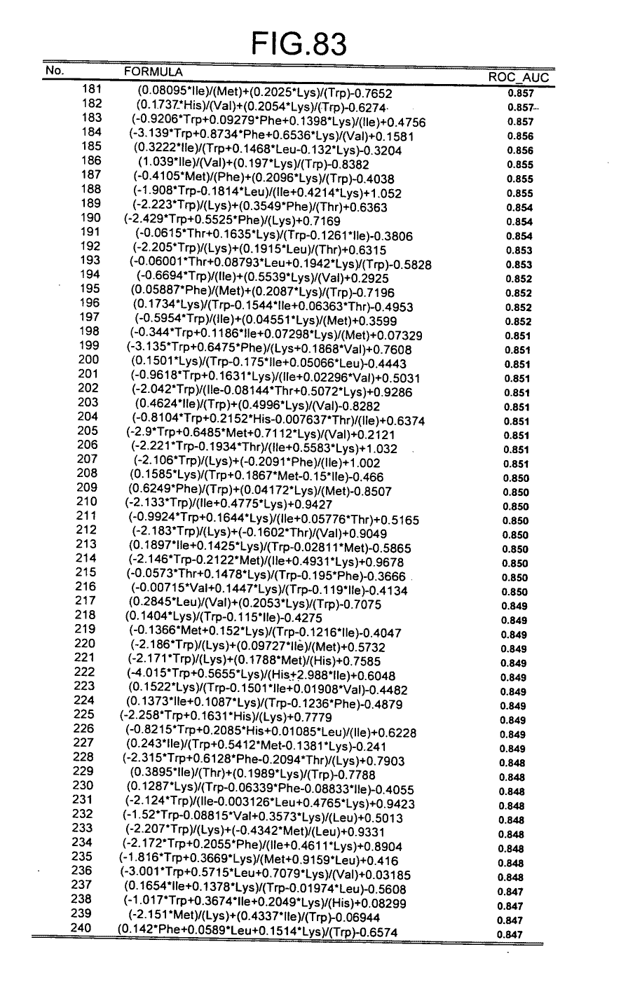

Furthermore, an evaluation using the AUC of the ROC curve is carried out for the discrimination between the 2 groups of the gastric cancer group and the gastric cancer-free group, and the AUC shows values larger than 0.7 for amino acid explanatory variables Thr, Asn, Val, Met, Tyr, Phe, His, Trp, and Arg (FIG. 79). Therefore, it is made clear that the amino acid explanatory variables Thr, Asn, Val, Met, Tyr, Phe, His, Trp, and Arg have an ability to discriminate between the 2 groups of the gastric cancer group and the gastric cancer-free group.

### Example 17

The sample data used in Example 16 is used. Using a method described in International Publication WO 2004/052191 Pamphlet that is an international application filed by the present applicant, indices by which the performance of discriminating between the 2 groups of the gastric cancer group and the gastric cancer-free group is maximized with regard to the discrimination of gastric cancer are eagerly searched, and an index formula 12 is obtained among a plurality of indices having an equivalent performance.

−6.272×Trp/Gln-0.08814×His/Glu Index formula 12

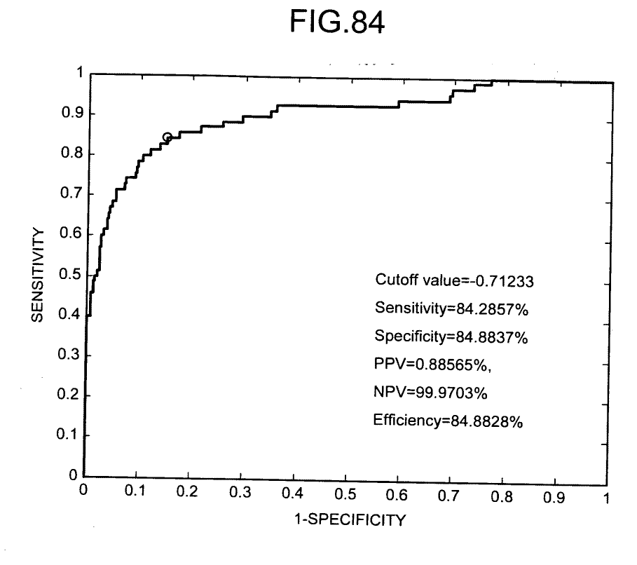

The performance for diagnosis of gastric cancer based on the index formula 12 is evaluated based on the AUC of the ROC curve (FIG. 84) in connection with the discrimination between the 2 groups of the gastric cancer group and the gastric cancer-free group, and an AUC of 0.905±0.022 (95% confidence interval: 0.860 to 0.950) is obtained. When the optimum cutoff value for the discrimination between the 2 groups of the gastric cancer group and the gastric cancer-free group by the index formula 12 is determined assuming that the symptom prevalence of the gastric cancer group is 0.16%, the cutoff value was −0.712, and a sensitivity of 84.3%, a specificity of 84.9%, a positive predictive value of 0.886%, a negative predictive value of 99.97%, and a correct diagnostic rate of 84.88% are obtained (FIG. 84). Thus, the index formula 12 is found to be a useful index with high diagnostic performance.

### Example 18

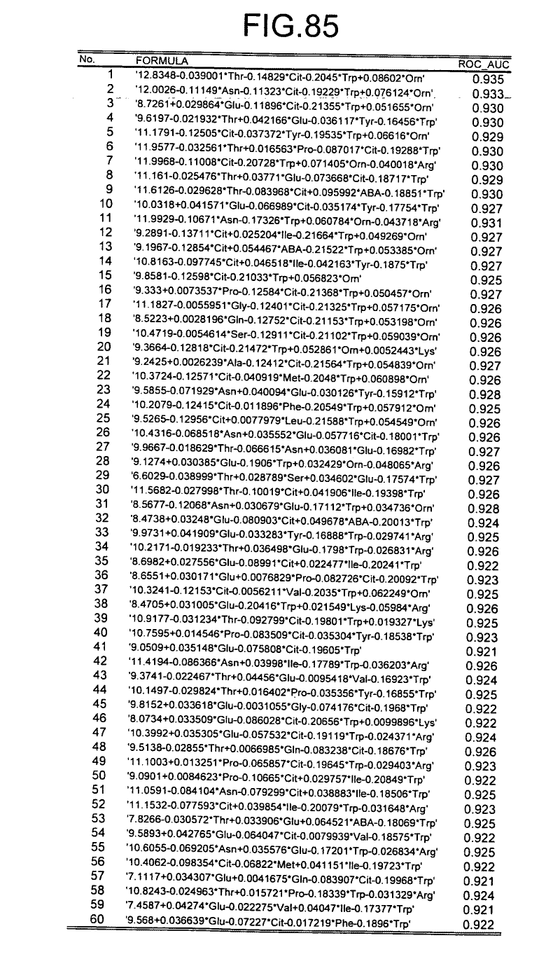

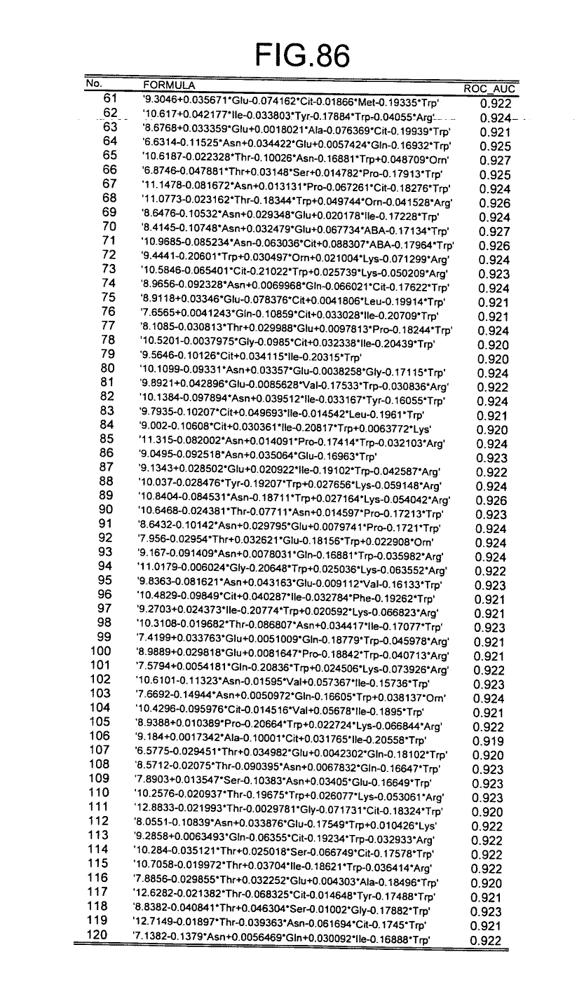

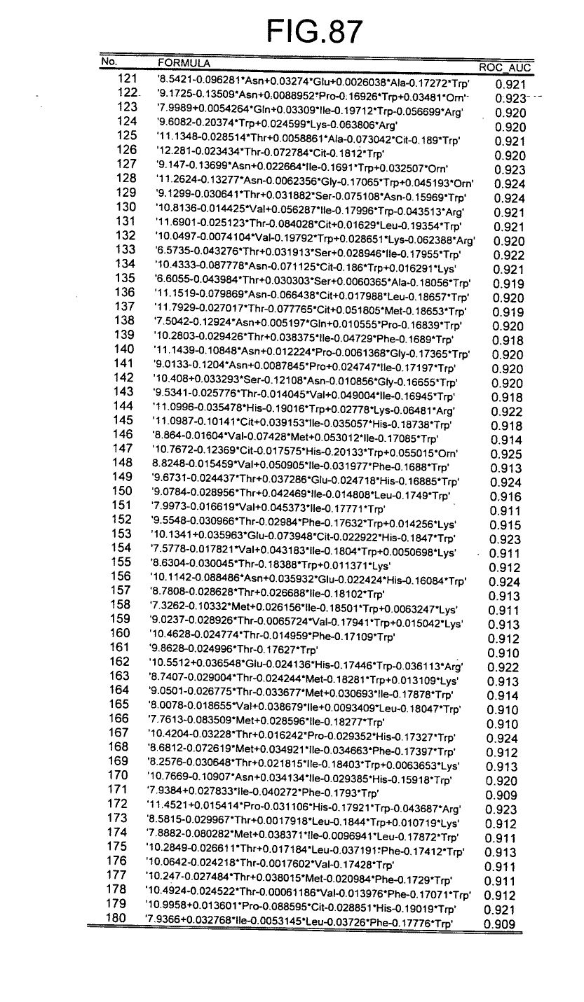

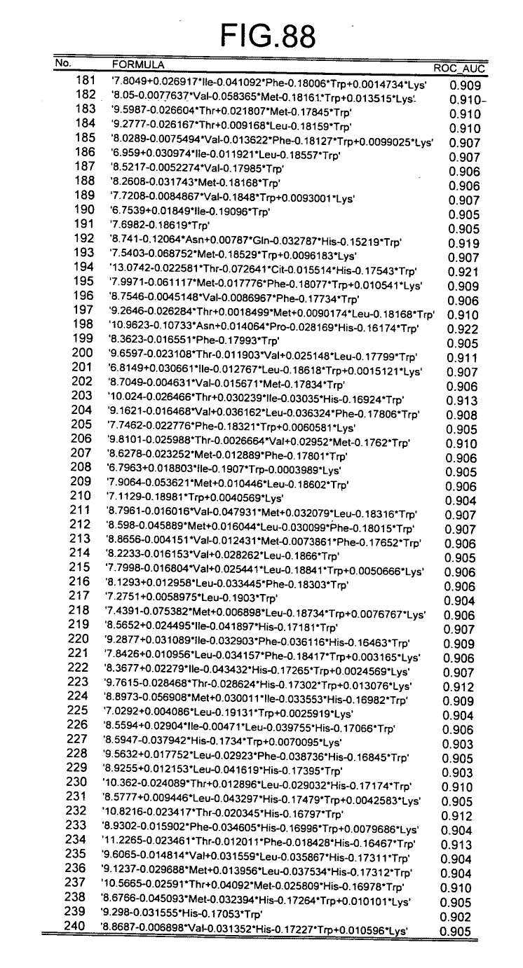

The sample data used in Example 16 is used. Indices by which the performance of discriminating between the 2 groups of the gastric cancer group and the gastric cancer-free group is maximized with regard to gastric cancer are searched by logistic analysis (explanatory variable coverage method based on the BIC minimum criterion), and a logistic regression equation composed of Val, Ile, His, and Trp (the numerical coefficients of the amino acid explanatory variables Val, Ile, His, and Trp, and the constant terms are, in the same order, −0.0149±0.0061, 0.0467±0.0148, −0.0296±0.0197, −0.1659±0.0233, and 9.182±1.467, respectively) is obtained as an index formula 13. In addition to that, a plurality of logistic regression equations having a discrimination performance equivalent to that of the index formula 11 is obtained. Those logistic regression equations are presented in FIG. 85, FIG. 86, FIG. 87, and FIG. 88. The respective values of the coefficients for the equations presented in FIG. 85, FIG. 86, FIG. 87, and FIG. 88 may be values multiplied by a real number.

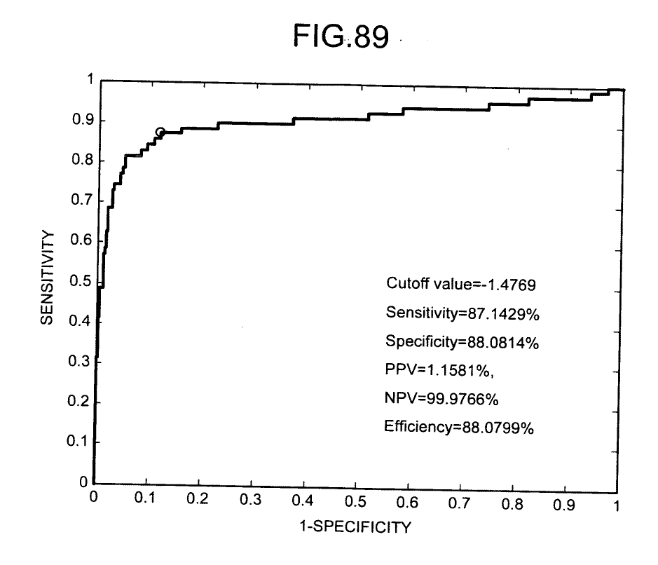

The performance for diagnosis of gastric cancer based on the index formula 13 is evaluated based on the AUC of the ROC curve (FIG. 89) in connection with the discrimination between the 2 groups of the gastric cancer group and the gastric cancer-free group, and an AUC of 0.909±0.027 (95% confidence interval: 0.857 to 0.961) is obtained. Thus, the index formula 13 is found to be a useful index with high diagnostic performance. When the optimum cutoff value for the discrimination between the 2 groups of the gastric cancer group and the gastric cancer-free group by the index formula 13 is determined assuming that the symptom prevalence of the gastric cancer group is 0.16%, the cutoff value is −1.477, and a sensitivity of 87.1%, a specificity of 88.1%, a positive predictive value of 1.16%, a negative predictive value of 99.98%, and a correct diagnostic rate of 88.08% are obtained (FIG. 89). Thus, the index formula 13 is found to be a useful index with high diagnostic performance.

### Example 19

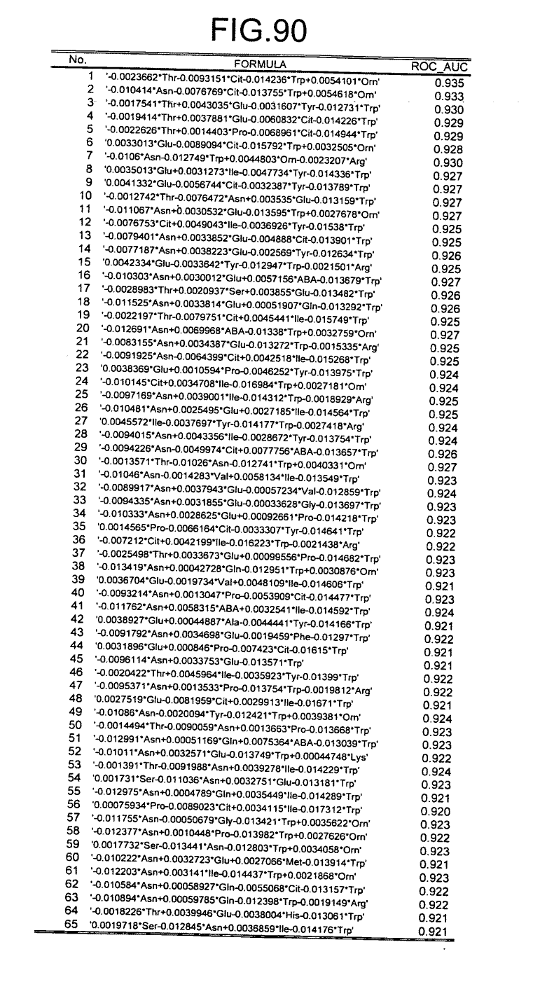

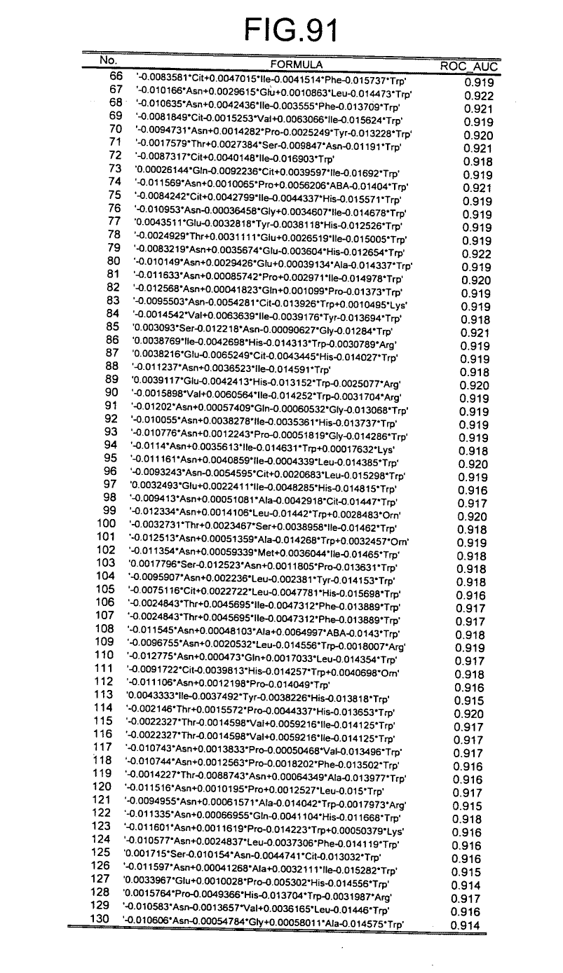

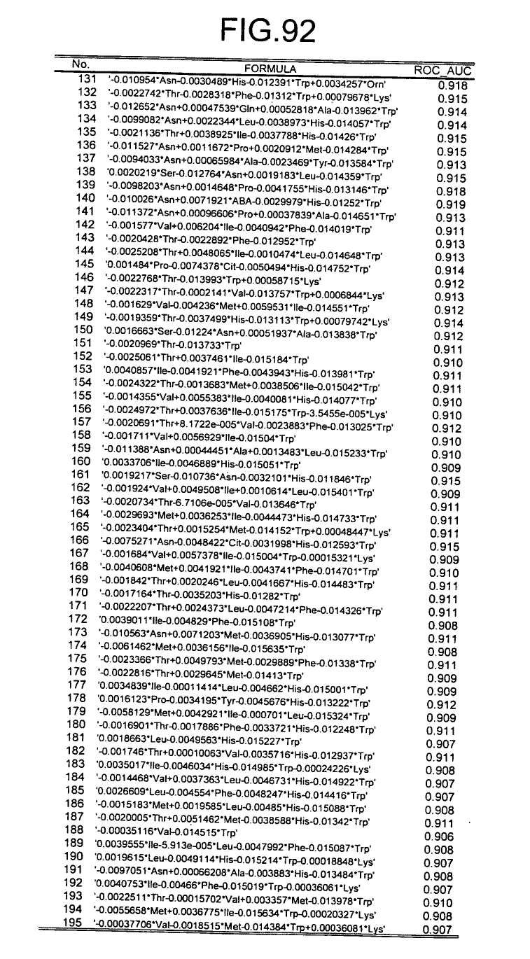

The sample data used in Example 16 is used. Indices by which the performance of discriminating between the 2 groups of the gastric cancer group and the gastric cancer-free group is maximized with regard to gastric cancer are searched by linear discriminant analysis (explanatory variable coverage method), and a linear discriminant function composed of Thr, Ile, His, and Trp (the numerical coefficients of the amino acid explanatory variables Thr, Ile, His, and Trp are, in the same order, −0.0021±−0.0011, 0.0039±−0.0018, −0.0038±−0.0023, and −0.0143±−0.0024, respectively) is obtained as an index formula 14. In addition to that, a plurality of linear discriminant functions having a discrimination performance equivalent to that of the index formula 14 is obtained. Those linear discriminant functions are presented in FIG. 90, FIG. 91, and FIG. 92. The respective values of the coefficients for the functions presented in FIG. 90, FIG. 91, and FIG. 92 may be values multiplied by a real number, or values obtained by adding an arbitrary constant term.

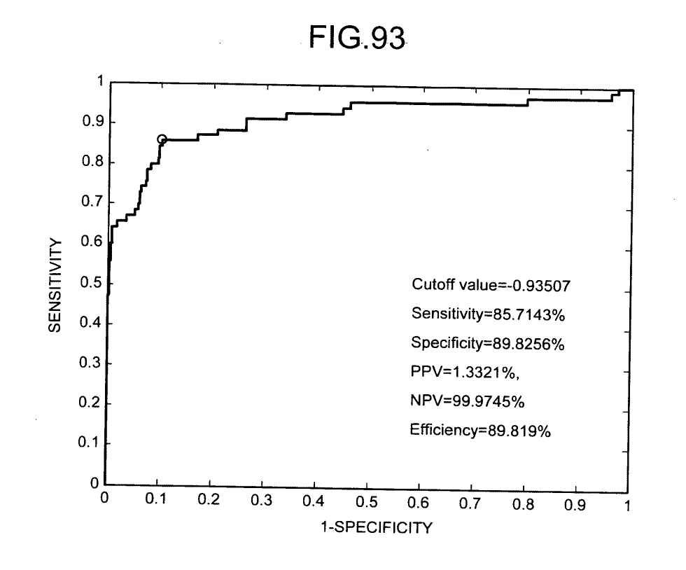

The performance for diagnosis of gastric cancer based on the index formula 14 is evaluated based on the AUC of the ROC curve (FIG. 93) in connection with the discrimination between the 2 groups of the gastric cancer group and the gastric cancer-free group, and an AUC of 0.914±0.024 (95% confidence interval: 0.867 to 0.962) is obtained. Thus, the index formula 14 is found to be a useful index with high diagnostic performance. When the optimum cutoff value for the discrimination between the 2 groups of the gastric cancer group and the gastric cancer-free group by the index formula 14 is determined assuming that the symptom prevalence of the gastric cancer group is 0.16%, the cutoff value is −0.935, and a sensitivity of 85.7%, a specificity of 89.8%, a positive predictive value of 1.33%, a negative predictive value of 99.97%, and a correct diagnostic rate of 89.82% are obtained (FIG. 93).

Thus, the index formula 14 is found to be a useful index with high diagnostic performance.

### Example 20

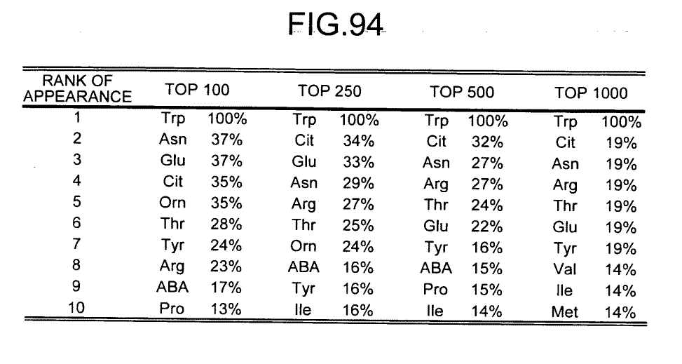

The sample data used in Example 16 is used. The areas under the ROC curve of all logistic regression equations for performing discrimination between the 2 groups of the gastric cancer group and the gastric cancer-free group with regard to gastric cancer, are calculated assuming that the maximum value of the amino acid explanatory variables appearing in each equation is 4 from among the used amino acid explanatory variables. Here, 10 kinds of amino acids are extracted in the decreasing appearance frequency order by the discrimination equations in which the areas under the ROC curve are in top 100, 250, 500, and 1000 in the respective combinations. As the amino acids whose appearance frequency is always in top 10 in the discrimination equations in which the areas under the ROC curve are in top 100, 250, 500, and 1000, Trp, Asn, Glu, Cit, Thr, Tyr, and Arg are extracted. Thus, it is made clear that multivariate discriminants using these amino acids as explanatory variables have an ability to discriminate between the 2 groups of the gastric cancer group and the gastric cancer-free group (FIG. 94).

Although the invention has been described with respect to specific embodiments for a complete and clear disclosure, the appended claims are not to be thus limited but are to be construed as embodying all modifications and alternative constructions that may occur to one skilled in the art that fairly fall within the basic teaching herein set forth.

What is claimed is:

1. A method of evaluating gastric cancer, comprising:
a measuring step of measuring amino acid concentration data on a concentration value of an amino acid in blood collected from a subject to be evaluated; and
a concentration value criterion evaluating step of evaluating a gastric cancer state in the subject based on the concentration value of at least one of Asn, Cys, His, Met, Orn, Phe, Trp, Pro, Lys, Leu, Glu, Arg, Ala, Thr, and Tyr contained in the amino acid concentration data of the subject measured at the measuring step.

2. The method of evaluating gastric cancer according to claim 1, wherein the concentration value criterion evaluating step further includes a concentration value criterion discriminating step of discriminating between gastric cancer and gastric cancer-free, discriminating a stage of gastric cancer, or discriminating the presence or absence of metastasis of gastric cancer to other organ in the subject based on the concentration value of at least one of Asn, Cys, His, Met, Orn, Phe, Trp, Pro, Lys, Leu, Glu, Arg, Ala, Thr, and Tyr contained in the amino acid concentration data of the subject measured at the measuring step.

3. The method of evaluating gastric cancer according to claim 1, wherein the concentration criterion evaluating step further includes:
a discriminant value calculating step of calculating a discriminant value that is a value of a multivariate discriminant with a concentration of the amino acid as an explanatory variable, based on both the concentration value of at least one of Asn, Cys, His, Met, Orn, Phe, Trp, Pro, Lys, Leu, Glu, Arg, Ala, Thr, and Tyr contained in the amino acid concentration data of the subject measured at the measuring step and the previously established multivariate discriminant; and
a discriminant value criterion evaluating step of evaluating the gastric cancer state in the subject based on the discriminant value calculated at the discriminant value calculating step,
wherein the multivariate discriminant contains at least one of Asn, Cys, His, Met, Orn, Phe, Trp, Pro, Lys, Leu, Glu, Arg, Ala, Thr, and Tyr as the explanatory variable.

4. The method of evaluating gastric cancer according to claim 3, wherein the discriminant value criterion evaluating step further includes a discriminant value criterion discriminating step of discriminating between gastric cancer and gastric cancer-free, discriminating a stage of gastric cancer, or discriminating the presence or absence of metastasis of gastric cancer to other organ in the subject based on the discriminant value calculated at the discriminant value calculating step.

5. The method of evaluating gastric cancer according to claim 4, wherein the multivariate discriminant is expressed by one fractional expression or the sum of a plurality of the fractional expressions and contains at least one of Asn, Cys, His, Met, Orn, Phe, Trp, Pro, Lys, Leu, Glu, Arg, Ala, Thr, and Tyr as the explanatory variable in any one of the numerator and denominator or both in the fractional expression constituting the multivariate discriminant.

6. The method of evaluating gastric cancer according to claim 5, wherein (a) when the discrimination between the gastric cancer and the gastric cancer-free is conducted at the discriminant value criterion discriminating step, the multivariate discriminant is formula 1, 2, or 3, (b) when the discrimination of the stage of gastric cancer is conducted at the discriminant value criterion discriminating step, the multivariate discriminant is formula 4, and (c) when the discrimination of the presence or absence of metastasis of gastric cancer to other organ is conducted at the discriminant value criterion discriminating step, the multivariate discriminant is formula 5:
a₁×Orn/(Trp+His)+b₁×(ABA+Ile)/Leu+c₁ (formula 1)
a₂×Glu/His+b₂×Ser/Trp+c₂×Arg/Pro+d₂ (formula 2)
a₃×Trp/Gln+b₃×His/Glu+c₃ (formula 3)
a₄×Gly/(Glu+Trp+Val)+b₄×Arg/His+c₄ (formula 4)
a₅×Ile/Glu+b₅×(Gly+Asn+Arg)/His+c₅ (formula 5)
wherein a₁ and b₁ in the formula 1 are arbitrary non-zero real numbers, c₁ in the formula 1 is arbitrary real number, a₂, b₂, and c₂ in the formula 2 are arbitrary non-zero real numbers, d₂ in the formula 2 is arbitrary real number, a₃ and b₃ in the formula 3 are arbitrary non-zero real numbers, c₃ in the formula 3 is arbitrary real number, a₄ and b₄ in the formula 4 are arbitrary non-zero real numbers, c₄ in the formula 4 is arbitrary real number, a₅ and b₅ in the formula 5 are arbitrary non-zero real numbers, and c₅ in the formula 5 is arbitrary real number.

7. The method of evaluating gastric cancer according to claim 4, wherein the multivariate discriminant is any one of a logistic regression equation, a linear discriminant, a multiple regression equation, a discriminant prepared by a support vector machine, a discriminant prepared by a Mahalanobis' generalized distance method, a discriminant prepared by canonical discriminant analysis, and a discriminant prepared by a decision tree.

8. The method of evaluating gastric cancer according to claim 7, wherein the multivariate discriminant is the logistic regression equation with Orn, Gln, Trp, and Cit as the explanatory variables, the linear discriminant with Orn, Gln, Trp, Phe, Cit, and Try as the explanatory variables, the logistic regression equation with Glu, Phe, His, and Trp as the explanatory variables, the linear discriminant with Glu, Pro, His, and Trp as the explanatory variables, the logistic regression equation with Val, Ile, His, and Trp as the explanatory variables, or the linear discriminant with Thr, Ile, His, and Trp as the explanatory variables.

9. A gastric cancer-evaluating apparatus comprising a control unit and a memory unit to evaluate a gastric cancer state in a subject to be evaluated, wherein the control unit includes:
a discriminant value-calculating unit that calculates a discriminant value that is a value of a multivariate discriminant with a concentration of an amino acid as an explanatory variable, based on both (a) a concentration value of at least one of Asn, Cys, His, Met, Orn, Phe, Trp, Pro, Lys, Leu, Glu, Arg, Ala, Thr, and Tyr contained in previously obtained amino acid concentration data of the subject on the concentration value of the amino acid and (b) the multivariate discriminant containing at least one of Asn, Cys, His, Met, Orn, Phe, Trp, Pro, Lys, Leu, Glu, Arg, Ala, Thr, and Tyr as the explanatory variable, stored in the memory unit; and
a discriminant value criterion-evaluating unit that evaluates the gastric cancer state in the subject based on the discriminant value calculated by the discriminant value-calculating unit.

10. The gastric cancer-evaluating apparatus according to claim 9, wherein the discriminant value criterion-evaluating unit further includes a discriminant value criterion-discriminating unit that discriminates between gastric cancer and gastric cancer-free, discriminates a stage of gastric cancer, or discriminates the presence or absence of metastasis of gastric cancer to other organ in the subject based on the discriminant value calculated by the discriminant value-calculating unit.

11. The gastric cancer-evaluating apparatus according to claim 10, wherein the multivariate discriminant is expressed by one fractional expression or the sum of a plurality of the fractional expressions and contains at least one of Asn, Cys, His, Met, Orn, Phe, Trp, Pro, Lys, Leu, Glu, Arg, Ala, Thr, and Tyr as the explanatory variable in any one of the numerator and denominator or both in the fractional expression constituting the multivariate discriminant.

12. The gastric cancer-evaluating apparatus according to claim 11, wherein (a) when the discrimination between the gastric cancer and the gastric cancer-free is conducted by the discriminant value criterion-discriminating unit, the multivariate discriminant is formula 1, 2, or 3, (b) when the discrimination of the stage of gastric cancer is conducted by the discriminant value criterion-discriminating unit, the multivariate discriminant is formula 4, and (c) when the discrimination of the presence or absence of metastasis of gastric cancer to other organ is conducted by the discriminant value criterion-discriminating unit, the multivariate discriminant is formula 5:
a₁×Orn/(Trp+His)+b₁×(ABA+Ile)/Leu+c₁ (formula 1)
a₂×Glu/His+b₂×Ser/Trp+c₂×Arg/Pro+d₂ (formula 2)
a₃×Trp/Gln+b₃×His/Glu+c₃ (formula 3)
a₄×Gly/(Glu+Trp+Val)+b₄×Arg/His+c₄ (formula 4)
a₅×Ile/Glu+b₅×(Gly+Asn+Arg)/His+c₅ (formula 5)
wherein a₁ and b₁ in the formula 1 are arbitrary non-zero real numbers, c₁ in the formula 1 is arbitrary real number, a₂, b₂, and c₂ in the formula 2 are arbitrary non-zero real numbers, d₂ in the formula 2 is arbitrary real number, a₃ and b₃ in the formula 3 are arbitrary non-zero real numbers, c₃ in the formula 3 is arbitrary real number, a₄ and b₄ in the formula 4 are arbitrary non-zero real numbers, c₄ in the formula 4 is arbitrary real number, a₅ and b₅ in the formula 5 are arbitrary non-zero real numbers, and c₅ in the formula 5 is arbitrary real number.

13. The gastric cancer-evaluating apparatus according to claim 10, wherein the multivariate discriminant is any one of a logistic regression equation, a linear discriminant, a multiple regression equation, a discriminant prepared by a support vector machine, a discriminant prepared by a Mahalanobis' generalized distance method, a discriminant prepared by canonical discriminant analysis, and a discriminant prepared by a decision tree.

14. The gastric cancer-evaluating apparatus according to claim 13, wherein the multivariate discriminant is the logistic regression equation with Orn, Gln, Trp, and Cit as the explanatory variables, the linear discriminant with Orn, Gln, Trp, Phe, Cit, and Try as the explanatory variables, the logistic regression equation with Glu, Phe, His, and Trp as the explanatory variables, the linear discriminant with Glu, Pro, His, and Trp as the explanatory variables, the logistic regression equation with Val, Ile, His, and Trp as the explanatory variables, or the linear discriminant with Thr, Ile, His, and Trp as the explanatory variables.

15. The gastric cancer-evaluating apparatus according to claim 9, wherein the control unit further includes a multivariate discriminant-preparing unit that prepares the multivariate discriminant stored in the memory unit, based on gastric cancer state information containing the amino acid concentration data and gastric cancer state index data on an index for indicating the gastric cancer state, stored in the memory unit,
wherein the multivariate discriminant-preparing unit further includes:
a candidate multivariate discriminant-preparing unit that prepares a candidate multivariate discriminant that is a candidate of the multivariate discriminant, based on a predetermined discriminant-preparing method from the gastric cancer state information;
a candidate multivariate discriminant-verifying unit that verifies the candidate multivariate discriminant prepared by the candidate multivariate discriminant-preparing unit, based on a predetermined verifying method; and
an explanatory variable-selecting unit that selects the explanatory variable of the candidate multivariate discriminant based on a predetermined explanatory variable-selecting method from a verification result obtained by the candidate multivariate discriminant-verifying unit, thereby selecting a combination of the amino acid concentration data contained in the gastric cancer state information used in preparing the candidate multivariate discriminant, and
wherein the multivariate discriminant-preparing unit prepares the multivariate discriminant by selecting the candidate multivariate discriminant used as the multivariate discriminant, from a plurality of the candidate multivariate discriminants, based on the verification results accumulated by repeatedly executing the candidate multivariate discriminant-preparing unit, the candidate multivariate discriminant-verifying unit, and the explanatory variable-selecting unit.

16. A gastric cancer-evaluating method of evaluating a gastric cancer state in a subject to be evaluated, the method is carried out with an information processing apparatus including a control unit and a memory unit, the method comprising:
(i) a discriminant value calculating step of calculating a discriminant value that is a value of a multivariate discriminant with a concentration of an amino acid as an explanatory variable, based on both (a) a concentration value of at least one of Asn, Cys, His, Met, Orn, Phe, Trp, Pro, Lys, Leu, Glu, Arg, Ala, Thr, and Tyr contained in previously obtained amino acid concentration data of the subject on the concentration value of the amino acid and (b) the multivariate discriminant containing at least one of Asn, Cys, His, Met, Orn, Phe, Trp, Pro, Lys, Leu, Glu, Arg, Ala, Thr, and Tyr as the explanatory variable, stored in the memory unit; and
(ii) a discriminant value criterion evaluating step of evaluating the gastric cancer state in the subject based on the discriminant value calculated at the discriminant value calculating step,
wherein the steps (i) and (ii) are executed by the control unit.

17. The gastric cancer-evaluating method according to claim 16, wherein the discriminant value criterion evaluating step further includes a discriminant value criterion discriminating step of discriminating between gastric cancer and gastric cancer-free, discriminating a stage of gastric cancer, or discriminating the presence or absence of metastasis of gastric cancer to other organ in the subject based on the discriminant value calculated at the discriminant value calculating step.

18. The gastric cancer-evaluating method according to claim 17, wherein the multivariate discriminant is expressed by one fractional expression or the sum of a plurality of the fractional expressions and contains at least one of Asn, Cys, His, Met, Orn, Phe, Trp, Pro, Lys, Leu, Glu, Arg, Ala, Thr, and Tyr as the explanatory variable in any one of the numerator and denominator or both in the fractional expression constituting the multivariate discriminant.

19. The gastric cancer-evaluating method according to claim 18, wherein (a) when the discrimination between the gastric cancer and the gastric cancer-free is conducted at the discriminant value criterion discriminating step, the multivariate discriminant is formula 1, 2, or 3, (b) when the discrimination of the stage of gastric cancer is conducted at the discriminant value criterion discriminating step, the multivariate discriminant is formula 4, and (c) when the discrimination of the presence or absence of metastasis of gastric cancer to other organ is conducted at the discriminant value criterion discriminating step, the multivariate discriminant is formula 5:
a₁×Orn/(Trp+His)+b₁×(ABA+Ile)/Leu+c₁ (formula 1)
a₂×Glu/His+b₂×Ser/Trp+c₂×Arg/Pro+d₂ (formula 2)
a₃×Trp/Gln+b₃×His/Glu+c₃ (formula 3)
a₄×Gly/(Glu+Trp+Val)+b₄×Arg/His+c₄ (formula 4)
a₅×Ile/Glu+b₅×(Gly+Asn+Arg)/His+c₅ (formula 5)
wherein a₁ and b₁ in the formula 1 are arbitrary non-zero real numbers, c₁ in the formula 1 is arbitrary real number, a₂, b₂, and c₂ in the formula 2 are arbitrary non-zero real numbers, d₂ in the formula 2 is arbitrary real number, a₃ and b₃ in the formula 3 are arbitrary non-zero real numbers, c₃ in the formula 3 is arbitrary real number, a₄ and b₄ in the formula 4 are arbitrary non-zero real numbers, c₄ in the formula 4 is arbitrary real number, a₅ and b₅ in the formula 5 are arbitrary non-zero real numbers, and c₅ in the formula 5 is arbitrary real number.

20. The gastric cancer-evaluating method according to claim 17, wherein the multivariate discriminant is any one of a logistic regression equation, a linear discriminant, a multiple regression equation, a discriminant prepared by a support vector machine, a discriminant prepared by a Mahalanobis' generalized distance method, a discriminant prepared by canonical discriminant analysis, and a discriminant prepared by a decision tree.

21. The gastric cancer-evaluating method according to claim 20, wherein the multivariate discriminant is the logistic regression equation with Orn, Gln, Trp, and Cit as the explanatory variables, the linear discriminant with Orn, Gln, Trp, Phe, Cit, and Try as the explanatory variables, the logistic regression equation with Glu, Phe, His, and Trp as the explanatory variables, the linear discriminant with Glu, Pro, His, and Trp as the explanatory variables, the logistic regression equation with Val, Ile, His, and Trp as the explanatory variables, or the linear discriminant with Thr, Ile, His, and Trp as the explanatory variables.

22. The gastric cancer-evaluating method according to claim 16, wherein the method further includes a multivariate discriminant preparing step of preparing the multivariate discriminant stored in the memory unit, based on gastric cancer state information containing the amino acid concentration data and gastric cancer state index date on an index for indicating the gastric cancer state, stored in the memory unit that is executed by the control unit, wherein the multivariate discriminant preparing step further includes:
a candidate multivariate discriminant preparing step of preparing a candidate multivariate discriminant that is a candidate of the multivariate discriminant, based on a predetermined discriminant-preparing method from the gastric cancer state information;
a candidate multivariate discriminant verifying step of verifying the candidate multivariate discriminant prepared at the candidate multivariate preparing step, based on a predetermined verifying method; and
an explanatory variable selecting step of selecting the explanatory variable of the candidate multivariate discriminant based on a predetermined explanatory variable-selecting method from a verification result obtained at the candidate multivariate discriminant verifying step, thereby selecting a combination of the amino acid concentration data contained in the gastric cancer state information used in preparing the candidate multivariate discriminant, and
wherein at the multivariate discriminant preparing step, the multivariate discriminant is prepared by selecting the candidate multivariate discriminant used as the multivariate discriminant from a plurality of the candidate multivariate discriminants, based on the verification results accumulated by repeatedly executing the candidate multivariate discriminant preparing step, the candidate multivariate discriminant verifying step, and the explanatory variable selecting step.

23. A gastric cancer-evaluating system comprising a gastric cancer-evaluating apparatus including a control unit and a memory unit to evaluate a gastric cancer state in a subject to be evaluated and an information communication terminal apparatus that provides amino acid concentration data of the subject on a concentration value of an amino acid connected to each other communicatively via a network,
wherein the information communication terminal apparatus includes:
an amino acid concentration data-sending unit that transmits the amino acid concentration data of the subject to the gastric cancer-evaluating apparatus; and
an evaluation result-receiving unit that receives an evaluation result of the gastric cancer state of the subject transmitted from the gastric cancer-evaluating apparatus,
wherein the control unit of the gastric cancer-evaluating apparatus includes:
an amino acid concentration data-receiving unit that receives the amino acid concentration data of the subject transmitted from the information communication terminal apparatus;
a discriminant value-calculating unit that calculates a discriminant value that is a value of a multivariate discriminant with a concentration of the amino acid as an explanatory variable, based on both (a) the concentration value of at least one of Asn, Cys, His, Met, Orn, Phe, Trp, Pro, Lys, Leu, Glu, Arg, Ala, Thr, and Tyr contained in the amino acid concentration data of the subject received by the amino acid concentration data-receiving unit and (b) the multivariate discriminant containing at least one of Asn, Cys, His, Met, Orn, Phe, Trp, Pro, Lys, Leu, Glu, Arg, Ala, Thr, and Tyr as the explanatory variable, stored in the memory unit;
a discriminant value criterion-evaluating unit that evaluates the gastric cancer state in the subject based on the discriminant value calculated by the discriminant value-calculating unit; and
an evaluation result-sending unit that transmits the evaluation result of the subject obtained by the discriminant value criterion-evaluating unit to the information communication terminal apparatus.

24. A gastric cancer-evaluating program product that makes an information processing apparatus including a control unit and a memory unit execute a method of evaluating a gastric cancer state in a subject to be evaluated, the method comprising:
(i) a discriminant value calculating step of calculating a discriminant value that is a value of a multivariate discriminant with a concentration of an amino acid as an explanatory variable, based on both (a) a concentration value of at least one of Asn, Cys, His, Met, Orn, Phe, Trp, Pro, Lys, Leu, Glu, Arg, Ala, Thr, and Tyr contained in previously obtained amino acid concentration data of the subject on the concentration value of the amino acid and (b) the multivariate discriminant containing at least one of Asn, Cys, His, Met, Orn, Phe, Trp, Pro, Lys, Leu, Glu, Arg, Ala, Thr, and Tyr as the explanatory variable, stored in the memory unit; and
(ii) a discriminant value criterion evaluating step of evaluating the gastric cancer state in the subject based on the discriminant value calculated at the discriminant value calculating step,
wherein the steps (i) and (ii) are executed by the control unit.

25. A computer-readable recording medium, comprising the gastric cancer-evaluating program product according to claim 24 recorded thereon.
